(19) United States
(12) Patent Application Publication
(10) Pub. No.: US 2010/0184805 A1
(43) Pub. Date: Jul. 22, 2010

(54) RENIN INHIBITORS

(75) Inventors: John J. Baldwin, Gwynedd Valley, PA (US); Salvacion Cacatian, Blue Bell, PA (US); David A. Claremon, Mple Glen, PA (US); Lawrence W. Dillard, Yardley, PA (US); Patrick T. Flaherty, Pittsburgh, PA (US); Alexey V. Ishchenko, Somerville, MA (US); Lanqi Jia, Horsham, PA (US); Gerard McGeehan, Garnet Valley, PA (US); Robert D. Simpson, Wilmington, DE (US); Suresh B. Singh, Kendall Park, NJ (US); Colin M. Tice, Ambler, PA (US); Zhenrong Xu, Horsham, PA (US); Jing Yuan, Lansdale, PA (US); Wei Zhao, Eagleville, PA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Vitae Pharmaceuticals, Inc.

(21) Appl. No.: 12/665,208

(22) PCT Filed: Jun. 20, 2008
(86) PCT No.: PCT/US08/07705

§ 371 Date: Feb. 5, 2010

Related U.S. Application Data
(60) Provisional application No. 60/936,375, filed on Jun. 20, 2007.

(51) Int. Cl. A61K 31/454; C07D 211/32; C07D 401/12; C07D 405/12; A61K 31/453; A61K 31/4545; A61K 31/445; A61P 43/00

(52) U.S. Cl. 514/316; 546/246; 546/190; 546/208; 546/207; 514/315; 514/326

(57) ABSTRACT

Disclosed are aspartic protease inhibitors represented by the following structural formula: and pharmaceutically acceptable salts thereof. These compounds are orally active and bind to aspartic proteases to inhibit their activity. They are useful in the treatment or amelioration of diseases associated with aspartic protease activity. The present invention is also directed to pharmaceutical compositions comprising a compound described herein or enantiomers, diastereomers, or salts thereof and a pharmaceutically acceptable carrier or excipient.

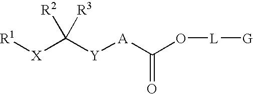

### RELATED APPLICATION

This application claims the benefit of U.S. Provisional Application No. 60/936,375, filed Jun. 20, 2007. The entire teachings of the above application are incorporated herein by reference.

### BACKGROUND OF THE INVENTION

Aspartic proteases, including renin, β-secretase (BACE), Candida albicans secreted aspartyl proteases, HIV protease, HTLV protease, and plasmepsins I and II, are implicated in a number of disease states. In hypertension, elevated levels of angiotensin I, the product of renin catalyzed cleavage of angiotensinogen are present. Elevated levels of β-amyloid, the product of BACE activity on amyloid precursor protein, are widely believed to be responsible for the amyloid plaques present in the brains of Alzheimer's disease patients. Secreted aspartyl proteases play a role in the virulence of the pathogen Candida albicans. The viruses HIV and HTLV depend on their respective aspartic proteases for viral maturation. Plasmodium falciparum uses plasmepsins I and II to degrade hemoglobin.

In the renin-angiotensin-aldosterone system (RAAS), the biologically active peptide angiotensin II (Ang II) is generated by a two-step mechanism. The highly specific aspartic protease renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAAS represents a major advance in the treatment of cardiovascular diseases (Zaman, M. A. et al Nature Reviews Drug Discovery 2002, 1, 621-636). ACE inhibitors and AT₁ blockers have been accepted as treatments of hypertension (Waeber B. et al., “The renin-angiotensin system: role in experimental and human hypertension”, in Berkenhager W. H., Reid J. L. (eds): Hypertension, Amsterdam, Elsevier Science Publishing Co, 1996, 489-519; Weber M. A., Am. J Hypertens., 1992, 5, 247S). In addition, ACE inhibitors are used for renal protection (Rosenberg M. E. et al., Kidney International, 1994, 45, 403; Breyer J. A. et al., Kidney International, 1994, 45, S156), in the prevention of congestive heart failure (Vaughan D. E. et al., Cardiovasc. Res., 1994, 28, 159; Fouad-Tarazi F. et al., Am. J. Med., 1988, 84 (Suppl. 3A), 83) and myocardial infarction (Pfeffer M. A. et al., N. Engl. J. Med, 1992, 327, 669).

Interest in the development of renin inhibitors stems from the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be bypassed by chymase, a serine protease (Husain A., J. Hypertens., 1993, 11, 1155). In patients, inhibition of ACE thus leads to bradykinin accumulation causing cough (5-20%) and potentially life-threatening angioneurotic edema (0.1-0.2%) (Israili Z. H. et al., Annals of Internal Medicine, 1992, 117, 234). Chymase is not inhibited by ACE inhibitors. Therefore, the formation of Ang II is still possible in patients treated with ACE inhibitors. Blockade of the ATI receptor (e.g., by losartan) on the other hand overexposes other AT-receptor subtypes to Ang II, whose concentration is dramatically increased by the blockade of ATI receptors. In summary, renin inhibitors are not only expected to be superior to ACE inhibitors and AT₁ blockers with regard to safety, but more importantly also with regard to their efficacy in blocking the RAAS.

Only limited clinical experience (Azizi M. et al., J. Hypertens., 1994, 12, 419; Neutel J. M. et al., Am. Heart, 1991, 122, 1094) has been generated with renin inhibitors because their peptidomimetic character imparts insufficient oral activity (Kleinert H. D., Cardiovasc. Drugs, 1995, 9, 645). The clinical development of several compounds has been stopped because of this problem together with the high cost of goods. It appears as though only one compound has entered clinical trials (Rahuel J. et al., Chem. Biol., 2000, 7, 493; Mealy N. E., Drugs of the Future, 2001, 26, 1139). Thus, metabolically stable, orally bioavailable and sufficiently soluble renin inhibitors that can be prepared on a large scale are not available. Recently, the first non-peptide renin inhibitors were described which show high in vitro activity (Oefner C. et al., Chem. Biol., 1999, 6, 127; Patent Application WO 97/09311; Maerki H. P. et al., Il Farmaco, 2001, 56, 21). The present invention relates to the unexpected identification of renin inhibitors of a non-peptidic nature and of low molecular weight. Orally active renin inhibitors, which are active in indications beyond blood pressure regulation where the tissular renin-chymase system may be activated leading to pathophysiologically altered local functions such as renal, cardiac and vascular remodeling, atherosclerosis, and restenosis, are described.

### SUMMARY OF THE INVENTION

Compounds have now been found which bind to aspartic proteases to inhibit their activity. They are useful in the treatment or amelioration of diseases associated with aspartic protease activity.

This invention provides compounds according to Formula Ia:

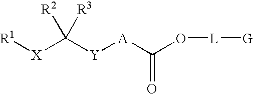

or an enantiomer, diastereomer, or pharmaceutically acceptable salt thereof; wherein:

R¹ is: a) (C₁-C₁₂)alkyl, (C₃-C₇)cycloalkyl, (C₄-C₁₂)cycloalkylalkyl, halo(C₁-C₁₂)alkyl, halo(C₃-C₇)cycloalkyl, halo(C₄-C₁₂)cycloalkylalkyl or saturated heterocyclyl, each optionally substituted with 1 to 5 groups independently selected from the group consisting of: halogen, (C₁-C₆)alkyl, halo(C₁-C₆)alkyl and oxo; or b) phenyl, napthyl, heteroaryl or bicyclic heteroaryl, each substituted with n groups represented by R¹¹, wherein n is an integer from 0 to 5, and wherein each R¹¹ is independently selected from the groups consisting of: 1) fluoride, chloride, bromide, iodide, cyano, nitro, amino, hydroxy, carboxy, (C₁-C₈)alkyl, (C₃-C₈)cycloalkyl, (C₁-C₃)alkyl(C₃-C₈)cycloalkyl, di(C₁-C₃)alkyl(C₃-C₈)cycloalkyl, (C₄-C₈)cycloalkylalkyl, (C₂-C₆)alkenyl, (C₅-C₈)cycloalkenyl, (C₅-C₈)cycloalkylalkenyl, (C₂-C₈)alkynyl, (C₃-C₈)cycloalkyl(C₂-C₄)alkynyl, halo(C₁-C₈)alkyl, halo(C₃-C₆)cycloalkyl, halo(C₄-C₈)cycloalkylalkyl, (C₁-C₃)alkyl(C₄-C₈)cycloalkylalkyl, di(C₁-C₃)alkyl(C₄-C₈)cycloalkylalkyl, halo(C₂-C₈)alkenyl, halo(C₅-C₈)cycloalkenyl, halo(C₆-C₈)cycloalkenylalkyl, halo(C₃-C₈)alkynyl, halo(C₅-C₈)cycloalkylalkynyl, (C₁-C₈)alkoxy, (C₃-C₈)cycloalkoxy, (C₄-C₈)cycloalkylalkoxy, (C₁-C₃)alkyl(C₃-C₈)cycloalkoxy, (C₁-C₃)alkyl(C₄-C₈)cycloalkylalkoxy, di(C₁-C₃)alkyl(C₃-C₈)-cycloalkoxy, di(C₁-C₃)alkyl(C₄-C₈)cycloalkylalkoxy, halo(C₁-C₆)alkoxy, halo(C₃-C₆)cycloalkoxy, halo(C₄-C₈)cycloalkylalkoxy, (C₁-C₈)alkylthio, (C₃-C₈)cycloalkylthio, (C₄-C₈)cycloalkylalkylthio, (C₁-C₃)alkyl(C₃-C₈)cycloalkylthio, (C₁-C₃)alkyl(C₄-C₈)cycloalkylalkylthio, di(C₁-C₃)alkyl(C₃-C₈)cycloalkylthio, di(C₁-C₃)alkyl(C₄-C₈)cycloalkylalkylthio, halo(C₁-C₈)alkylthio, halo(C₃-C₈)cycloalkylthio, halo(C₄-C₈)cycloalkylalkylthio, (C₁-C₈)alkanesulfinyl, (C₃-C₈)cycloalkanesulfinyl, (C₄-C₈)cycloalkylalkanesulfinyl, (C₁-C₃)alkyl(C₃-C₈)cycloalkanesulfinyl, (C₁-C₃)alkyl(C₄-C₈)cycloalkylalkanesulfinyl, di(C₁-C₃)alkyl(C₃-C₈)cycloalkanesulfinyl, di(C₁-C₃)alkyl(C₄-C₈)cycloalkylalkanesulfinyl, halo(C₁-C₈)alkanesulfinyl, halo(C₃-C₈)cycloalkanesulfinyl, halo(C₄-C₈)cycloalkylalkanesulfinyl, (C₁-C₈)alkanesulfonyl, (C₃-C₈)cycloalkanesulfonyl, (C₄-C₈)cycloalkylalkanesulfonyl, (C₁-C₃)alkyl(C₃-C₈)cycloalkanesulfonyl, (C₁-C₃)alkyl(C₄-C₈)cycloalkylalkanesulfonyl, di(C₁-C₃)alkyl(C₃-C₈)cycloalkanesulfonyl, di(C₁-C₃)alkyl(C₄-C₈)cycloalkylalkanesulfonyl, halo(C₁-C₈)alkanesulfonyl, halo(C₃-C₈)cycloalkanesulfonyl, halo(C₄-C₈)cycloalkylalkanesulfonyl, (C₁-C₈)alkylamino, di(C₁-C₈)alkylamino, (C₁-C₆)alkoxy(C₁-C₆)alkoxy, halo(C₁-C₆)alkoxy(C₁-C₆)alkoxy, (C₁-C₈)alkoxycarbonyl, aminocarbonyl, (C₁-C₈)alkylaminocarbonyl, di(C₁-C₈)alkylaminocarbonyl, piperidino, pyrrolidino, cyano(C₁-C₆)alkyl, hydroxy(C₁-C₆)alkyl, carboxy(C₁-C₆)alkyl, (C₁-C₈)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkylthio(C₁-C₆)alkyl, halo(C₄-C₈)cycloalkylalkylthio(C₁-C₆)alkyl, (C₁-C₈)alkanesulfinyl(C₁-C₆)alkyl, (C₃-C₈)cycloalkanesulfinyl(C₁-C₆)alkyl, (C₄-C₈)cycloalkylalkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkanesulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkanesulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl, (C₁-C₈)acylamino(C₁-C₆)alkyl, piperidino(C₁-C₆)alkyl, pyrrolidino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, or di(C₁-C₈)alkylaminocarboxy(C₁-C₆)alkyl; and 2) phenyl, napthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, bicyclic heteroaryl(C₁-C₃)alkyl, phenyl(C₁-C₃)alkoxy, naphthyl(C₁-C₃)alkoxy, heteroaryl(C₁-C₃)alkoxy, and bicyclic heteroaryl(C₁-C₃)alkoxy, each optionally substituted with 1 to 5 groups independently selected from the group consisting of: fluoride, chloride, cyano, (C₁-C₆)alkyl, halo(C₁-C₆)alkyl, (C₁-C₆)alkoxy, halo(C₁-C₆)alkoxy, (C₁-C₆)alkanesulfonyl, (C₁-C₆)alkoxycarbonyl and aminocarbonyl. With the proviso that when R¹ is imidazole, then R³ is not H or C₁-C₆ alkyl.

X and Y are each independently —CH₂— or a single bond.

R² is a substituted or unsubstituted (C₁-C₁₂)alkyl, (C₂-C₁₂)alkenyl, (C₂-C₁₂)alkynyl, (C₁-C₁₂)alkoxy, (C₂-C₁₂)alkenyloxy, (C₁-C₁₂)alkylthio, (C₁-C₆)alkoxy(C₁-C₆)alkyl, (C₁-C₆)alkylthio(C₁-C₆)alkyl, (C₁-C₆)alkoxy(C₁-C₆)alkoxy, (C₁-C₆)alkoxy(C₁-C₆)alkylthio, (C₁-C₆)alkylthio(C₁-C₆)alkoxy, (C₁-C₆)alkylthio(C₁-C₆)alkylthio, (C₁-C₄)alkoxy(C₁-C₄)alkoxy(C₁-C₄)alkyl, aminocarbonylamino(C₁-C₁₂)alkyl, aminocarbonylamino(C₁-C₁₂)alkoxy, aminocarbonylamino(C₁-C₁₂)alkylthio, (C₁-C₆)alkanoylamino(C₁-C₆)alkyl, (C₁-C₆)alkanoylamino(C₁-C₆)alkoxy, (C₁-C₆)alkanoylamino(C₁-C₆)alkylthio, (C₃-C₄)cycloalkylcarbonylamino(C₁-C₆)alkyl, (C₃-C₄)cycloalkylcarbonylamino(C₁-C₆)alkoxy, (C₃-C₄)cycloalkylcarbonylamino(C₁-C₆)alkylthio, aminosulfonylamino(C₁-C₁₂)alkyl, aminosulfonylamino(C₁-C₁₂)alkoxy, aminosulfonylamino(C₁-C₁₂)alkylthio, (C₁-C₆)alkanesulfonylamino(C₁-C₆)alkyl, (C₁-C₆)alkane-sulfonylamino(C₁-C₆)-alkoxy, (C₁-C₆)alkanesulfonylamino(C₁-C₆)alkylthio, formylamino(C₁-C₆)alkyl, formylamino(C₁-C₆)alkoxy, formylamino(C₁-C₆)alkylthio, (C₁-C₆)alkoxycarbonylamino(C₁-C₆)alkyl, (C₁-C₆)alkoxycarbonylamino(C₁-C₆)alkoxy, (C₁-C₆)alkoxycarbonylamino(C₁-C₆)alkylthio, (C₁-C₆)alkylaminocarbonylamino(C₁-C₆)alkyl, di(C₁-C₆)alkylaminocarbonylamino(C₁-C₆)alkyl, (C₁-C₆)alkylaminocarbonylamino(C₁-C₆)alkoxy, di(C₁-C₆)alkylaminocarbonylamino(C₁-C₆)alkoxy, (C₁-C₆)alkylaminocarbonylamino(C₁-C₆)alkylthio, di(C₁-C₆)alkylaminocarbonylamino(C₁-C₆)alkylthio, aminocarbonyl(C₁-C₆)alkyl, aminocarbonyl(C₁-C₆)alkoxy, aminocarbonyl(C₁-C₆)alkylthio, (C₁-C₆)alkylaminocarbonyl(C₁-C₆)alkyl, (C₁-C₆)alkylaminocarbonyl(C₁-C₆)alkoxy, (C₁-C₆)alkylaminocarbonyl(C₁-C₆)alkylthio, aminocarboxy(C₁-C₆)alkyl, aminocarboxy(C₁-C₆)alkoxy, aminocarboxy(C₁-C₆)alkylthio, (C₁-C₆)alkylaminocarboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₁₂)alkoxycarbonylamino, (C₁-C₁₂)alkylaminocarbonylamino, (C₁-C₈)oxoalkyl, or (C₁-C₁₂)alkanoylamino; wherein each group represented by R² is substituted by 0 to 6 groups selected from: halogen, cyano, hydroxyl, (C₁-C₃)alkoxy, (C₃-C₆)cycloalkyl, (C₃-C₆)cycloalkoxy, halo(C₁-C₃)alkoxy, halo(C₃-C₆)cycloalkyl or halo(C₃-C₆)cycloalkoxy; wherein any thio-moiety of said unsubstituted or substituted R² group is optionally and independently replaced by —S(O)— or —S(O)₂—; and wherein any carbonyl moiety of said unsubstituted or substituted R² group is optionally and independently replaced by a thiocarbonyl moiety.

R³ is: a) —H, halogen, (C₁-C₆)alkyl, (C₁-C₆)alkoxy, hydroxyl, hydroxy(C₁-C₆)alkyl, hydroxy(C₁-C₆)alkoxy, (C₁-C₆)alkanoylamino, (C₁-C₆)alkoxycarbonylamino, (C₁-C₆)alkylaminocarbonylamino, di(C₁-C₆)alkylaminocarbonylamino, (C₁-C₆)alkanesulfonylamino, (C₁-C₆)alkylaminosulfonylamino, or di(C₁-C₆)alkylaminosulfonylamino; or b) phenylamino or heteroarylamino in which each phenylamino or heteroarylamino group is optionally substituted with 1 to 5 groups independently selected from the group consisting of: fluoride, chloride, bromide, iodide, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, and di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkylalkylthio-(C₁-C₆)alkyl, halo(C₁-C₈)alkylthio(C₁-C₆)alkyl, halo(C₃-C₈)cycloalkylthio(C₁-C₆)alkyl, halo(C₄-C₈)cycloalkylalkylthio(C₁-C₆)alkyl, (C₁-C₈)alkanesulfinyl(C₁-C₆)alkyl, (C₃-C₈)cycloalkanesulfinyl(C₁-C₆)alkyl, (C₄-C₈)cycloalkylalkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkanesulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkanesulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, and di(C₁-C₈)alkylaminocarboxy(C₁-C₆)alkyl.

With the proviso that: 1)when R³ is hydroxyl, halogen or optionally substituted phenylamino or heteroarylamino, then R² is not a substituted or unsubstituted (C₁-C₁₂)alkoxy, (C₁-C₆)alkoxy(C₁-C₆)alkoxy, (C₁-C₆)alkylthio(C₁-C₆)alkoxy, aminocarbonylamino(C₁-C₁₂)alkyl, aminocarbonylamino(C₁-C₁₂)alkoxy, (C₁-C₆)alkanoylamino(C₁-C₆)alkoxy, (C₃-C₄)cycloalkylcarbonylamino(C₁-C₆)alkoxy, aminosulfonylamino(C₁-C₁₂)alkoxy, (C₁-C₆)alkanesulfonylamino(C₁-C₆)alkoxy, formylamino(C₁-C₆)alkoxy, (C₁-C₆)alkoxycarbonylamino(C₁-C₆)alkoxy, (C₁-C₆)alkylaminocarbonylamino(C₁-C₆)alkoxy, di(C₁-C₆)alkylaminocarbonylamino(C₁-C₆)alkoxy, aminocarbonyl(C₁-C₆)alkoxy, (C₁-C₆)alkylaminocarbonyl(C₁-C₆)alkoxy, aminocarboxy(C₁-C₆)alkoxy, (C₁-C₆)alkylaminocarboxy(C₁-C₆)alkoxy, (C₁-C₁₂)alkoxycarbonylamino, (C₁-C₁₂)alkylaminocarbonylamino, or (C₁-C₁₂)alkanoylamino; 2) when R³ is hydroxyl, halogen, or optionally substituted phenylamino or heteroarylamino, then R² is not a unsubstituted or substituted (C₁-C₁₂)alkylthio, (C₁-C₆)alkoxy(C₁-C₆)alkylthio, (C₁-C₆)alkylthio(C₁-C₆)alkylthio, aminocarbonylamino(C₁-C₁₂)alkylthio, (C₁-C₆)alkanoylamino(C₁-C₆)alkylthio, (C₃-C₄)cycloalkylcarbonylamino(C₁-C₆)alkylthio, aminosulfonylamino(C₁-C₁₂)alkylthio, (C₁-C₆)alkanesulfonylamino(C₁-C₆)alkylthio, formylamino(C₁-C₆)alkylthio, (C₁-C₆)alkoxycarbonylamino(C₁-C₆)alkylthio, (C₁-C₆)alkylaminocarbonylamino(C₁-C₆)alkylthio, di(C₁-C₆)alkylaminocarbonylamino(C₁-C₆)alkylthio, aminocarbonyl(C₁-C₆)alkylthio, (C₁-C₆)alkylaminocarbonyl(C₁-C₆)alkylthio, aminocarboxy(C₁-C₆)alkylthio or (C₁-C₆)alkylaminocarboxy(C₁-C₆)alkylthio, wherein the thio-moiety is replaced by —S(O)— or —S(O)₂—; and 3) when R³ is hydroxyl, halogen, or optionally substituted phenylamino or heteroarylamino, then R² is not a unsubstituted or substituted aminocarbonylamino(C₁-C₁₂)alkoxy, aminocarbonylamino(C₁-C₁₂)alkylthio, (C₁-C₆)alkanoylamino(C₁-C₆)alkoxy, (C₁-C₆)alkanoylamino(C₁-C₆)alkylthio, (C₃-C₄)cycloalkylcarbonylamino(C₁-C₆)alkoxy, (C₃-C₄)cycloalkylcarbonylamino(C₁-C₆)alkylthio, formylamino(C₁-C₆)alkoxy, formylamino(C₁-C₆)alkylthio, (C₁-C₆)alkoxycarbonylamino(C₁-C₆)alkoxy, (C₁-C₆)alkoxycarbonylamino(C₁-C₆)alkylthio, (C₁-C₆)alkylaminocarbonylamino(C₁-C₆)alkoxy, di(C₁-C₆)alkylaminocarbonylamino(C₁-C₆)alkoxy, (C₁-C₆)alkylaminocarbonylamino(C₁-C₆)alkylthio, di(C₁-C₆)alkylaminocarbonylamino(C₁-C₆)alkylthio, aminocarbonyl(C₁-C₆)alkoxy, aminocarbonyl(C₁-C₆)alkylthio, (C₁-C₆)alkylaminocarbonyl-(C₁-C₆)alkoxy, (C₁-C₆)alkylaminocarbonyl(C₁-C₆)alkylthio, aminocarboxy(C₁-C₆)alkoxy, aminocarboxy(C₁-C₆)alkylthio, (C₁-C₆)alkylaminocarboxy(C₁-C₆)alkoxy, (C₁-C₆)alkylaminocarboxy(C₁-C₆)alkylthio, (C₁-C₁₂)alkoxycarbonylamino, (C₁-C₁₂)alkylaminocarbonylamino, or (C₁-C₁₂)alkanoylamino, wherein the carbonyl moiety is replaced by a thiocarbonyl moiety.

A is a saturated or unsaturated 4-, 5-, 6-, or 7-membered ring which is optionally bridged by (CH₂)ₘ via bonds to two members of said ring, wherein said ring is composed of carbon atoms, and 0-2 hetero atoms selected from 0, 1, or 2 nitrogen atoms, 0 or 1 oxygen atoms, and 0 or 1 sulfur atoms, said ring being optionally substituted with up to four moieties independently selected from the group consisting of: halogen, (C₁-C₆)alkyl, halo(C₁-C₆)alkyl, and oxo; m is 1 to 3; and the carbonyl carbon and Y are attached to carbon or nitrogen atoms in ring A in a 1,2-, 1,3- or 1,4-relationship.

L is: 1) a linear (C₂-C₄)alkyl chain when G is —OH, —OR⁹, —NH₂, —NHR⁹, —NR⁹R¹⁰, —NHC(═NH)NH₂, or —NHC(═NH)NHR⁹; or 2) a linear (C₁-C₃)alkyl chain when G is —C(═NH)NH₂ or —C(═NH)NHR⁹; or one or more of the carbon atoms of L may be part of a 3-, 4-, 5-, 6-, or 7-membered saturated ring composed of carbon atoms, and 0-2 hetero atoms selected from 0 or 1 nitrogen atoms, 0 or 1 oxygen atoms, and 0 or 1 sulfur atoms. L is substituted by 1-4 groups independently selected from R⁵, R⁶, R⁷, and R⁸.

Each R⁵, R⁶, R⁷, and R⁸ is independently selected from: 1) hydrogen; 2) (C₁-C₁₂)alkyl, (C₃-C₈)cycloalkyl, (C₃-C₈)cycloalkyl(C₁-C₃)alkyl, (C₂-C₁₂)alkenyl, (C₅-C₈)cycloalkyl(C₁-C₃)alkenyl, (C₂-C₁₂)alkynyl, (C₃-C₈)cycloalkyl(C₁-C₃)alkynyl, (C₄-C₁₂)bicycloalkyl(C₁-C₃)alkyl, (C₈-C₁₄)tricycloalkyl(C₁-C₃)alkyl, (C₁-C₆)alkoxy(C₁-C₆)alkyl, (C₃-C₈)cycloalkoxy(C₁-C₃)alkyl, (C₁-C₆)alkylthio(C₁-C₆)alkyl, (C₃-C₈)cycloalkylthio(C₁-C₃)alkyl, saturated heterocyclyl, and saturated heterocyclyl(C₁-C₃)alkyl; wherein each R⁵, R⁶, R⁷ and R⁸ is optionally and independently substituted by a group selected from: halogen, cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, halo(C₃-C₆)cycloalkoxy; and divalent sulfur atoms are optionally oxidized to sulfoxide or sulfone; and 3) phenyl, naphthyl, heteroaryl, phenyl(C₁-C₃)alkyl, naphthyl(C₁-C₃)alkyl, and heteroaryl(C₁-C₃)alkyl; each optionally substituted with 1 to 3 groups independently selected from: fluoride, chloride, bromide, iodide, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkythio(C₁-C₆)alkyl, halo(C₄-C₈)-cycloalkylalkylthio(C₁-C₆)alkyl, (C₁-C₈)alkanesulfinyl(C₁-C₆)alkyl, (C₃-C₈)cycloalkanesulfinyl(C₁-C₆)alkyl, (C₄-C₈)cycloalkylalkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkanesulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkanesulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, di(C₁-C₈)alkylaminocarboxy(C₁-C₆)alkyl, phenyl, napthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, napthyl(C₁-C₃)alkyl, heteroaryl(C₁-C₃)alkyl, and bicyclic heteroaryl(C₁-C₃)alkyl; wherein the aromatic and heteroaromatic groups are optionally substituted with 1 to 3 groups independently selected from: fluoride, chloride, cyano, (C₁-C₃)alkyl, halo(C₁-C₃)alkyl, (C₁-C₃)alkoxy, halo(C₁-C₃)-alkoxy, (C₁-C₃)alkanesulfonyl, and (C₁-C₃)alkoxycarbonyl.

G is —OH, —OR⁹, —NH₂, —NHR⁹, —NR⁹R¹⁰, —C(═NH)NH₂, —C(═NH)NHR⁹, —NHC(═NH)NH₂, or —NHC(═NH)NHR⁹.

R⁹ is: a) (C₁-C₁₂)alkyl, (C₄-C₁₂)cycloalkylalkyl, halo(C₁-C₁₂)alkyl, halo(C₄-C₁₂)cycloalkylalkyl, (C₂-C₁₂)alkenyl, (C₅-C₁₂)cycloalkylalkenyl, halo(C₂-C₁₂)alkenyl, halo(C₅-C₁₂)cycloalkylalkenyl, (C₂-C₁₂)alkynyl, (C₅-C₁₂)cycloalkylalkynyl, halo(C₂-C₁₂)alkynyl, halo(C₅-C₁₂)cycloalkylalkynyl, (C₁-C₆)alkoxy(C₁-C₆)alkyl, halo(C₁-C₆)alkoxy(C₁-C₆)alkyl, (C₁-C₆)alkylthio(C₁-C_{C}₆)alkyl, halo(C₁-C₆)alkylthio(C₁-C₆)alkyl, (C₁-C₆)alkanesulfinyl(C₁-C₆)alkyl, halo(C₁-C₆)alkanesulfinyl(C₁-C₆)alkyl, (C₁-C₆)alkanesulfonyl(C₁-C₆)alkyl, halo(C₁-C₆)alkanesulfonyl(C₁-C₆)alkyl, aminocarbonyl(C₁-C₆)alkyl, (C₁-C₆)alkylaminocarbonyl(C₁-C₆)alkyl, di(C₁-C₆)alkylaminocarbonyl(C₁-C₆)alkyl, cyano(C₁-C₆)alkyl, carboxy(C₁-C₆)alkyl, (C₁-C₆)alkoxycarbonyl(C₁-C₆)alkyl, saturated heterocyclyl, or saturated heterocyclyl(C₁-C₆)alkyl; or b) phenyl, naphthyl, heteroaryl, phenyl(C₁-C₃)alkyl, naphthyl(C₁-C₃)alkyl, or heteroaryl(C₁-C₃)alkyl, each optionally and independently substituted by 1 to 3 groups selected from: 1) fluoride, chloride, bromide, iodide, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkylthio(C₁-C₆)alkyl, halo(C₄-C₈)cycloalkylalkylthio(C₁-C₆)alkyl, (C₁-C₈)alkanesulfinyl(C₁-C₆)alkyl, (C₃-C₈)cycloalkanesulfinyl(C₁-C₆)alkyl, (C₄-C₈)cycloalkylalkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkanesulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkanesulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl, (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl and di(C₁-C₈)alkylaminocarboxy(C₁-C₆)alkyl; and 2) phenyl, napthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, napthyl(C₁-C₃)alkyl, heteroaryl(C₁-C₃)alkyl, and bicyclic heteroaryl(C₁-C₃)alkyl, each optionally substituted with 1 to 3 groups independently selected from: fluoride, chloride, cyano, (C₁-C₃)alkyl, halo(C₁-C₃)alkyl, (C₁-C₃)alkoxy, halo(C₁-C₃)alkoxy, (C₁-C₃)alkanesulfonyl, and (C₁-C₃)-alkoxycarbonyl.

Alternatively, R⁹ taken together with one of R⁵, R⁶, R⁷ or R⁸ and their intervening atoms form a saturated 3-, 4-, 5-, 6-, or 7-membered “L-G ring” comprising 3 to 7 carbon atoms, and 1 or 2 heteroatoms selected from 0 or 1 nitrogen atoms, 0 or 1 oxygen atoms, and 0 or 1 sulfur atoms; said L-G ring being optionally substituted with 1 to 4 groups selected from: halogen, (C₁-C₈)alkyl, halo(C₁-C₈)alkyl, (C₃-C₈)cycloalkyl, (C₁-C₂)alkyl(C₃-C₆)cycloalkyl, halo(C₃-C₈)cycloalkyl, hydroxy(C₃-C₈)cycloalkyl, (C₃-C₈)cycloalkyl(C₁-C₃)alkyl, halo(C₃-C₈)cycloalkyl(C₁-C₃)alkyl, hydroxy(C₃-C₈)cycloalkyl(C₁-C₃)alkyl, (C₁-C₈)alkoxy, halo(C₁-C₈)alkoxy, (C₃-C₈)cycloalkoxy, halo(C₃-C₈)cycloalkoxy, hydroxy(C₃-C₈)cycloalkoxy, (C₁-C₈)alkoxy(C₁-C₃)alkyl, halo(C₁-C₈)alkoxy(C₁-C₃)alkyl, (C₃-C₈)cycloalkoxy(C₁-C₃)alkyl, halo(C₃-C₈)cycloalkoxy(C₁-C₃)alkyl, hydroxy(C₃-C₈)cycloalkoxy(C₁-C₃)alkyl, (C₃-C₈)cycloalkyl(C₁-C₃)alkoxy(C₁-C₃)alkyl, halo(C₃-C₈)cycloalkyl(C₁-C₃)alkoxy(C₁-C₃)alkyl, hydroxy(C₃-C₈)cycloalkyl(C₁-C₃)alkoxy(C₁-C₃)alkyl, (C₁-C₈)alkylthio, halo(C₁-C₈)alkylthio, (C₃-C₈)cycloalkylthio, halo(C₃-C₈)cycloalkylthio, hydroxy(C₃-C₈)cycloalkylthio, (C₃-C₈)cycloalkyl(C₁-C₃)alkylthio, halo(C₃-C₈)cycloalkyl(C₁-C₃)alkylthio, hydroxy(C₃-C₈)cycloalkyl(C₁-C₃)alkylthio, (C₁-C₈)alkylthio(C₁-C₃)alkyl, halo(C₁-C₈)alkylthio(C₁-C₃)alkyl, (C₃-C₈)cycloalkylthio(C₁-C₃)alkyl, halo(C₃-C₈)cycloalkylthio(C₁-C₃)alkyl, hydroxy(C₃-C₈)cycloalkylthio(C₁-C₃)alkyl, (C₃-C₈)cycloalkyl(C₁-C₃)alkylthio(C₁-C₃)alkyl, halo(C₃-C₈)cycloalkyl(C₁-C₃)alkylthio(C₁-C₃)alkyl, hydroxy(C₃-C₈)cycloalkyl(C₁-C₃)alkylthio(C₁-C₃)alkyl and oxo.

R¹⁰ is (C₁-C₆)alkyl or halo(C₁-C₆)alkyl.

In another embodiment, the present invention is directed to pharmaceutical compositions comprising a compound described herein or an enantiomer, diastereomer, or salt thereof and a pharmaceutically acceptable carrier or excipient.

In another embodiment, the present invention is directed to a method of antagonizing aspartic protease inhibitors in a subject in need thereof comprising administering to the subject a therapeutically effective amount of a compound described herein or an enantiomer, diastereomer, or salt thereof.

In another embodiment, the present invention is directed to method for treating or ameliorating an aspartic protease mediated disorder in a subject in need thereof comprising administering to said subject a therapeutically effective amount of a compound described herein or an enantiomer, diastereomer, or salt thereof.

In another embodiment, the present invention is directed to a method for treating or ameliorating a renin mediated disorder in a subject in need thereof comprising administering to the subject an effective amount of a compound described herein or an enantiomer, diastereomer, or salt thereof.

In another embodiment, the present invention is directed to a method for the treatment of hypertension in a subject in need thereof comprising administering to the subject a compound described herein in combination therapy with one or more additional agents said additional agent selected from the group consisting of α-blockers, β-blockers, calcium channel blockers, diuretics, angiotensin converting enzyme (ACE) inhibitors, dual ACE and neutral endopeptidase (NEP) inhibitors, angiotensin-receptor blockers (ARBs), aldosterone synthase inhibitors, aldosterone-receptor antagonists, and endothelin receptor antagonists.

### DETAILED DESCRIPTION OF THE INVENTION

A description of embodiments of a compound of Formula la of the invention follows. It is understood that the invention encompasses all combinations of the substituent variables (i.e., R¹, R², R³, etc.) defined herein. Values and particular values for the variables in Formula la are provided in the following paragraphs.

R¹ is a) (C₁-C₁₂)alkyl, (C₃-C₇)cycloalkyl, (C₄-C₁₂)cycloalkylalkyl, halo(C₁-C₁₂)alkyl, halo(C₃-C₇)cycloalkyl, halo(C₄-C₁₂)cycloalkylalkyl or saturated heterocyclyl, each optionally substituted with 1 to 5 groups independently selected from the group consisting of: halogen, (C₁-C₆)alkyl, halo(C₁-C₆)alkyl and oxo; or b) phenyl, napthyl, heteroaryl or bicyclic heteroaryl, each substituted with n groups represented by R¹¹. With the proviso that when R¹ is imidazole, then R³ is not H or C₁-C₆ alkyl.

In a particular embodiment of this invention, R¹ is a) (C₁-C₉)alkyl, (C₃-C₇)cycloalkyl, (C₄-C₉)cycloalkylalkyl, halo(C₁-C₉)alkyl, halo(C₃-C₇)cycloalkyl, halo(C₄-C₉)cycloalkylalkyl, or saturated heterocyclyl each optionally substituted with 1 to 3 groups independently selected from: fluoride, (C₁-C₃)alkyl, halo(C₁-C₃)alkyl, and oxo; or b) phenyl, napthyl, heteroaryl or bicyclic heteroaryl, each substituted with n groups represented by R¹¹,

In another particular embodiment, R¹ is a saturated heterocycle, phenyl, or heteroaryl; wherein the saturated heterocycle is substituted with n groups, represented by R¹¹. In a further particular embodiment of this invention, R¹ is a phenyl group optionally substituted with 1 to 4 R¹¹ substituents. In another particular embodiment, R¹ is phenyl optionally substituted with 0-3 groups independently selected from fluoride, chloride and methyl.

R² is a substituted or unsubstituted (C₁-C₁₂)alkyl, (C₂-C₁₂)alkenyl, (C₂-C₁₂)alkynyl, (C₁-C₁₂)alkoxy, (C₂-C₁₂)alkenyloxy, (C₁-C₁₂)alkylthio, (C₁-C₆)alkoxy(C₁-C₆)alkyl, (C₁-C₆)alkylthio(C₁-C₆)alkyl, (C₁-C₆)alkoxy(C₁-C₆)alkoxy, (C₁-C₆)alkoxy(C₁-C₆)alkylthio, (C₁-C₆)alkylthio(C₁-C₆)alkoxy, (C₁-C₆)alkylthio(C₁-C₆)alkylthio, (C₁-C₄)alkoxy(C₁-C₄)alkoxy(C₁-C₄)alkyl, aminocarbonylamino(C₁-C₁₂)alkyl, aminocarbonylamino(C₁-C₁₂)alkoxy, aminocarbonylamino(C₁-C₁₂)alkylthio, (C₁-C₆)alkanoylamino(C₁-C₆)alkyl, (C₁-C₆)alkanoylamino(C₁-C₆)alkoxy, (C₁-C₆)alkanoylamino(C₁-C₆)alkylthio, (C₃-C₄)cycloalkylcarbonylamino(C₁-C₆)alkyl, (C₃-C₄)cycloalkylcarbonylamino(C₁-C₆)alkoxy, (C₃-C₄)cycloalkylcarbonylamino(C₁-C₆)alkylthio, aminosulfonylamino(C₁-C₁₂)alkyl, aminosulfonylamino(C₁-C₁₂)alkoxy, aminosulfonylamino(C₁-C₁₂)alkylthio, (C₁-C₆)alkanesulfonylamino(C₁-C₆)alkyl, (C₁-C₆)alkane-sulfonylamino(C₁-C₆)-alkoxy, (C₁-C₆)alkanesulfonylamino(C₁-C₆)alkylthio, formylamino(C₁-C₆)alkyl, formylamino(C₁-C₆)alkoxy, formylamino(C₁-C₆)alkylthio, (C₁-C₆)alkoxycarbonylamino(C₁-C₆)alkyl, (C₁-C₆)alkoxycarbonylamino(C₁-C₆)alkoxy, (C₁-C₆)alkoxycarbonylamino(C₁-C₆)alkylthio, (C₁-C₆)alkylaminocarbonylamino(C₁-C₆)alkyl, di(C₁-C₆)alkylaminocarbonylamino(C₁-C₆)alkyl, (C₁-C₆)alkylaminocarbonylamino(C₁-C₆)alkoxy, di(C₁-C₆)alkylaminocarbonylamino(C₁-C₆)alkoxy, (C₁-C₆)alkylaminocarbonylamino(C₁-C₆)alkylthio, di(C₁-C₆)alkylaminocarbonylamino(C₁-C₆)alkylthio, aminocarbonyl(C₁-C₆)alkyl, aminocarbonyl(C₁-C₆)alkoxy, aminocarbonyl(C₁-C₆)alkylthio, (C₁-C₆)alkylaminocarbonyl(C₁-C₆)alkyl, (C₁-C₆)alkylaminocarbonyl(C₁-C₆)alkoxy, (C₁-C₆)alkylaminocarbonyl(C₁-C₆)alkylthio, aminocarboxy(C₁-C₆)alkyl, aminocarboxy(C₁-C₆)alkoxy, aminocarboxy(C₁-C₆)alkylthio, (C₁-C₆)alkylaminocarboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₁₂)alkoxycarbonylamino, (C₁-C₁₂)alkylaminocarbonylamino, (C₁-C₈)oxoalkyl, or (C₁-C₁₂)alkanoylamino; wherein each group represented by R² is substituted by 0 to 6 groups selected from: halogen, cyano, hydroxyl, (C₁-C₃)alkoxy, (C₃-C₆)cycloalkyl, (C₃-C₆)cycloalkoxy, halo(C₁-C₃)alkoxy, halo(C₃-C₆)cycloalkyl or halo(C₃-C₆)cycloalkoxy; wherein any thio-moiety of said unsubstituted or substituted R² group is optionally and independently replaced by —S(O)— or —S(O)₂—; and wherein any carbonyl moiety of said unsubstituted or substituted R² group is optionally and independently replaced by a thio-carbonyl moiety.

In a particular embodiment of the present invention, R² is (C₁-C₈)alkyl, (C₄-C₈)cycloalkylalkyl, fluoro(C₁-C₈)alkyl, fluoro(C₄-C₈)cycloalkylalkyl, (C₁-C₈)alkoxy, (C₄-C₈)cycloalkylalkoxy, fluoro(C₁-C₈)alkoxy, hydroxy(C₁-C₈)alkyl, (C₁-C₅)alkoxy(C₁-C₅)alkyl, halo(C₁-C₅)alkylamino(C₁-C₅)alkyl, (C₁-C₅)alkoxy(C₁-C₅)hydroxyalkyl, (C₃-C₄)cycloalkoxy(C₁-C₅)alkyl, fluoro(C₁-C₅)alkoxy(C₁-C₅)alkyl, fluoro(C₃-C₄)cycloalkoxy(C₁-C₅)alkyl, (C₁-C₅)alkylthio(C₁-C₅)alkyl, (C₁-C₅)alkoxy(C₁-C₅)alkoxy, hydroxy(C₁-C₈)alkoxy, (C₃-C₄)cycloalkoxy(C₁-C₅)alkoxy, fluoro(C₁-C₅)alkoxy(C₁-C₅)alkoxy, fluoro(C₃-C₄)cycloalkoxy(C₁-C₅)alkoxy, (C₁-C₃)alkoxy(C₁-C₃)alkoxy(C₁-C₃)alkyl, fluoro(C₁-C₃)-alkoxy(C₁-C₃)alkoxy(C₁-C₃)alkyl, aminocarbonylamino(C₁-C₈)alkyl, aminocarbonylamino(C₁-C₈)alkoxy, (C₁-C₅)alkanoylamino(C₁-C₅)alkyl, (C₁-C₅)alkanoylamino(C₁-C₅)alkoxy, fluoro(C₁-C₅)alkanoylamino(C₁-C₅)alkyl, fluoro(C₁-C₅)alkanoylamino(C₁-C₅)alkoxy, (C₁-C₃)alkoxy(C₁-C₅)alkanoylamino(C₁-C₅)alkyl, (C₁-C₃)alkoxy(C₁-C₅)alkanoylamino(C₁-C₅)alkoxy, (C₃-C₄)cycloalkylcarbonylamino(C₁-C₅)alkyl, (C₃-C₄)cycloalkylcarbonylamino(C₁-C₅)alkoxy, aminosulfonylamino(C₁-C₈)alkyl, aminosulfonylamino(C₁-C₈)alkoxy, (C₁-C₅)alkanesulfonylamino(C₁-C₅)alkyl, (C₁-C₅)alkanesulfonylamino(C₁-C₅)alkoxy, formylamino(C₁-C₅)alkyl, formylamino(C₁-C₅)alkoxy, (C₁-C₅)alkoxycarbonylamino(C₁-C₅)alkyl, (C₁-C₅)alkoxycarbonylamino(C₁-C₅)alkoxy, (C₁-C₅)alkylaminocarbonylamino(C₁-C₅)alkyl, (C₁-C₅)alkylaminocarbonylamino(C₁-C₅)alkyl, di(C₁-C₅)alkylaminocarbonylamino(C₁-C₅)alkoxy, aminocarbonyl(C₁-C₅)alkyl, aminocarbonyl(C₁-C₅)alkoxy, (C₁-C₅)alkylaminocarbonyl(C₁-C₅)alkyl, (C₁-C₅)alkylaminocarbonyl(C₁-C₅)alkoxy, aminocarboxy(C₁-C₅)alkyl, aminocarboxy(C₁-C₅)alkoxy, (C₁-C₅)alkylaminocarboxy(C₁-C₅)alkyl, (C₁-C₅)alkylaminocarboxy(C₁-C₅)alkoxy, (C₁-C₈)alkoxycarbonylamino, (C₁-C₈)alkylaminocarbonylamino, (C₁-C₈)alkanoylamino, fluoro(C₁-C₈)alkoxycarbonylamino, fluoro(C₁-C₈)alkylaminocarbonylamino, (C₁-C₈)oxoalkyl, or fluoro(C₁-C₈)alkanoylamino.

In another particular embodiment, R² is (C₁-C₅)alkoxy(C₁-C₅)alkyl, (C₁-C₅)alkoxy(C₁-C₅)hydroxyalkyl, (C₃-C₄)cycloalkoxy(C₁-C₅)alkyl, fluoro(C₁-C₅)alkoxy(C₁-C₅)alkyl, (C₁-C₅)alkoxy(C₁-C₅)alkoxy, fluoro(C₁-C₅)alkoxy(C₁-C₅)alkoxy, (C₁-C₃)alkoxy(C₁-C₃)alkoxy(C₁-C₃)alkyl, fluoro(C₁-C₃)alkoxy(C₁-C₃)alkoxy(C₁-C₃)alkyl, (C₁-C₅)alkanoylamino(C₁-C₅)alkyl, (C₁-C₅)alkanoylamino(C₁-C₅)alkoxy, fluoro(C₁-C₅)alkanoylamino(C₁-C₅)alkyl, fluoro(C₁-C₅)alkanoylamino(C₁-C₅)alkoxy, (C₁-C₃)alkoxy(C₁-C₅)alkanoylamino(C₁-C₅)alkyl, (C₁-C₃)alkoxy(C₁-C₅)alkanoylamino(C₁-C₅)alkoxy, (C₁-C₅)alkoxycarbonylamino(C₁-C₅)alkyl, (C₁-C₅)alkoxycarbonylamino(C₁-C₅)alkoxy, (C₁-C₅)alkylaminocarbonylamino(C₁-C₅)alkyl, di(C₁-C₅)alkylaminocarbonylamino(C₁-C₅)alkoxy, (C₁-C₈)alkylaminocarbonylamino, fluoro(C₁-C₈)alkoxycarbonylamino, or (C₁-C₈)oxoalkyl.

In further particular embodiment of the present invention, R² is 4-methoxybutyl, 3-(methoxycarbonylamino)propyl or 2-(methoxycarbonylamino)ethoxy.

R³ is a) —H, halogen, (C₁-C₆)alkyl, (C₁-C₆)alkoxy, hydroxyl, hydroxy(C₁-C₆)alkyl, hydroxy(C₁-C₆)alkoxy, (C₁-C₆)alkanoylamino, (C₁-C₆)alkoxycarbonylamino, (C₁-C₆)alkylaminocarbonylamino, di(C₁-C₆)alkylaminocarbonylamino, (C₁-C₆)alkanesulfonylamino, (C₁-C₆)alkylaminosulfonylamino, or di(C₁-C₆)alkylaminosulfonylamino; or b) phenylamino or heteroarylamino in which each phenylamino or heteroarylamino group is optionally substituted with 1 to 5 groups independently selected from the group consisting of: fluoride, chloride, bromide, iodide, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, and di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkylalkylthio-(C₁-C₆)alkyl, halo(C₁-C₈)alkylthio(C₁-C₆)alkyl, halo(C₃-C₈)cycloalkylthio(C₁-C₆)alkyl, halo(C₄-C₈)cycloalkylalkylthio(C₁-C₆)alkyl, (C₁-C₈)alkanesulfinyl(C₁-C₆)alkyl, (C₃-C₈)cycloalkanesulfinyl(C₁-C₆)alkyl, (C₄-C₈)cycloalkylalkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkanesulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkanesulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, and di(C₁-C₈)alkylaminocarboxy(C₁-C₆)alkyl.

It is provided that: 1) when R³ is hydroxyl, halogen or optionally substituted phenylamino or heteroarylamino, then R² is not a substituted or unsubstituted (C₁-C₁₂)alkoxy, (C₁-C₆)alkoxy(C₁-C₆)alkoxy, (C₁-C₆)alkylthio(C₁-C₆)alkoxy, aminocarbonylamino(C₁-C₁₂)alkyl, aminocarbonylamino(C₁-C₁₂)alkoxy, (C₁-C₆)alkanoylamino(C₁-C₆)alkoxy, (C₃-C₄)cycloalkylcarbonylamino(C₁-C₆)alkoxy, aminosulfonylamino(C₁-C₁₂)alkoxy, (C₁-C₆)alkanesulfonylamino(C₁-C₆)alkoxy, formylamino(C₁-C₆)alkoxy, (C₁-C₆)alkoxycarbonylamino(C₁-C₆)alkoxy, (C₁-C₆)alkylaminocarbonylamino(C₁-C₆)alkoxy, di(C₁-C₆)alkylaminocarbonylamino(C₁-C₆)alkoxy, aminocarbonyl(C₁-C₆)alkoxy, (C₁-C₆)alkylaminocarbonyl(C₁-C₆)alkoxy, aminocarboxy(C₁-C₆)alkoxy, (C₁-C₆)alkylaminocarboxy(C₁-C₆)alkoxy, (C₁-C₁₂)alkoxycarbonylamino, (C₁-C₁₂)alkylaminocarbonylamino, or (C₁-C₁₂)alkanoylamino; 2) when R³ is hydroxyl, halogen, or optionally substituted phenylamino or heteroarylamino, then R² is not a unsubstituted or substituted (C₁-C₁₂)alkylthio, (C₁-C₆)alkoxy(C₁-C₆)alkylthio, (C₁-C₆)alkylthio(C₁-C₆)alkylthio, aminocarbonylamino(C₁-C₁₂)alkylthio, (C₁-C₆)alkanoylamino(C₁-C₆)alkylthio, (C₃-C₄)cycloalkylcarbonylamino(C₁-C₆)alkylthio, aminosulfonylamino(C₁-C₁₂)alkylthio, (C₁-C₆)alkanesulfonylamino(C₁-C₆)alkylthio, formylamino(C₁-C₆)alkylthio, (C₁-C₆)alkoxycarbonylamino(C₁-C₆)alkylthio, (C₁-C₆)alkylaminocarbonylamino(C₁-C₆)alkylthio, di(C₁-C₆)alkylaminocarbonylamino(C₁-C₆)alkylthio, aminocarbonyl(C₁-C₆)alkylthio, (C₁-C₆)alkylaminocarbonyl(C₁-C₆)alkylthio, aminocarboxy(C₁-C₆)alkylthio or (C₁-C₆)alkylaminocarboxy(C₁-C₆)alkylthio, wherein the thio-moiety is replaced by —S(O)— or —S(O)₂—; and 3) when R³ is hydroxyl, halogen, or optionally substituted phenylamino or heteroarylamino, then R² is not a unsubstituted or substituted aminocarbonylamino(C₁-C₁₂)alkoxy, aminocarbonylamino(C₁-C₁₂)alkylthio, (C₁-C₆)alkanoylamino(C₁-C₆)alkoxy, (C₁-C₆)alkanoylamino(C₁-C₆)alkylthio, (C₃-C₄)cycloalkylcarbonylamino(C₁-C₆)alkoxy, (C₃-C₄)cycloalkylcarbonylamino(C₁-C₆)alkylthio, formylamino(C₁-C₆)alkoxy, formylamino(C₁-C₆)alkylthio, (C₁-C₆)alkoxycarbonylamino(C₁-C₆)alkoxy, (C₁-C₆)alkoxycarbonylamino(C₁-C₆)alkylthio, (C₁-C₆)alkylaminocarbonylamino(C₁-C₆)alkoxy, di(C₁-C₆)alkylaminocarbonylamino(C₁-C₆)alkoxy, (C₁-C₆)alkylaminocarbonylamino(C₁-C₆)alkylthio, di(C₁-C₆)alkylaminocarbonylamino(C₁-C₆)alkylthio, aminocarbonyl(C₁-C₆)alkoxy, aminocarbonyl(C₁-C₆)alkylthio, (C₁-C₆)alkylaminocarbonyl-(C₁-C₆)alkoxy, (C₁-C₆)alkylaminocarbonyl(C₁-C₆)alkylthio, aminocarboxy(C₁-C₆)alkoxy, aminocarboxy(C₁-C₆)alkylthio, (C₁-C₆)alkylaminocarboxy(C₁-C₆)alkoxy, (C₁-C₆)alkylaminocarboxy(C₁-C₆)alkylthio, (C₁-C₁₂)alkoxycarbonylamino, (C₁-C₁₂)alkylaminocarbonylamino, or (C₁-C₁₂)alkanoylamino, wherein the carbonyl moiety is replaced by a thiocarbonyl moiety.

In a more particular embodiment, R³ is —H, halogen, —OH, (C₁-C₄)alkanoylamino, or (C₁-C₃)alkoxy. In a further particular embodiment of the present invention, R³ is hydrogen, fluoride, chloride, —OH or (C₁-C₃)alkoxy. In a more particular embodiment, R³ is —H or —OH.

X and Y are each independently —CH₂— or a single bond. In a more particular embodiment, X and Y are both single bonds.

A is a saturated or unsaturated 4-, 5-, 6-, or 7-membered ring which is optionally bridged by (CH₂)ₘ via bonds to two members of said ring, wherein said ring is composed of carbon atoms, and 0-2 hetero atoms selected from 0, 1, or 2 nitrogen atoms, 0 or 1 oxygen atoms, and 0 or 1 sulfur atoms, said ring being optionally substituted with up to four moieties independently selected from the group consisting of: halogen, (C₁-C₆)alkyl, halo(C₁-C₆)alkyl, and oxo; where m is 1 to 3; and where the carbonyl carbon and Y are attached to carbon or nitrogen atoms in ring A in a 1,2-, 1,3- or 1,4-relationship. Preferably, the orientation of attachment to ring A of Y and the carbonyl carbon is in a 1,3-relationship.

In a particular embodiment, A is:

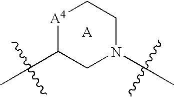

wherein A⁴ is CH₂, O, CH₂CH₂, or CH₂O, and the rest of the variables are as described for Formula I.

L is: 1) a linear (C₂-C₄)alkyl chain when G is —OH, —OR⁹, —NH₂, —NHR⁹, —NR⁹R¹⁰, —NHC(═NH)NH₂, or —NHC(═NH)NHR⁹; or 2) a linear (C₁-C₃)alkyl chain when G is —C(═NH)NH₂ or —C(═NH)NHR⁹; or 3) one or more of the carbon atoms of L may be part of a 3-, 4-, 5-, 6-, or 7-membered saturated ring composed of carbon atoms, and 0-2 hetero atoms selected from 0 or 1 nitrogen atoms, 0 or 1 oxygen atoms, and 0 or 1 sulfur atoms. L is substituted by 1-4 groups independently selected from R⁵, R⁶, R⁷, and R⁸.

Each R⁵, R⁶, R⁷, and R⁸ is independently selected from: 1) hydrogen; 2) (C₁-C₁₂)alkyl, (C₃-C₈)cycloalkyl, (C₃-C₈)cycloalkyl(C₁-C₃)alkyl, (C₂-C₁₂)alkenyl, (C₅-C₈)cycloalkyl(C₁-C₃)alkenyl, (C₂-C₁₂)alkynyl, (C₃-C₈)cycloalkyl(C₁-C₃)alkynyl, (C₄-C₁₂)bicycloalkyl(C₁-C₃)alkyl, (C₈-C₁₄)tricycloalkyl(C₁-C₃)alkyl, (C₁-C₆)alkoxy(C₁-C₆)alkyl, (C₃-C₈)cycloalkoxy(C₁-C₃)alkyl, (C₁-C₆)alkylthio(C₁-C₆)alkyl, (C₃-C₈)cycloalkylthio(C₁-C₃)alkyl, saturated heterocyclyl, and saturated heterocyclyl(C₁-C₃)alkyl; wherein each R⁵, R⁶, R⁷ and R⁸ is optionally and independently substituted by a group selected from: halogen, cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, halo(C₃-C₆)cycloalkoxy; and divalent sulfur atoms are optionally oxidized to sulfoxide or sulfone; and 3) phenyl, naphthyl, heteroaryl, phenyl(C₁-C₃)alkyl, naphthyl(C₁-C₃)alkyl, and heteroaryl(C₁-C₃)alkyl; each optionally substituted with 1 to 3 groups independently selected from: fluoride, chloride, bromide, iodide, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkythio(C₁-C₆)alkyl, halo(C₄-C₈)-cycloalkylalkylthio(C₁-C₆)alkyl, (C₁-C₈)alkanesulfinyl(C₁-C₆)alkyl, (C₃-C₈)cycloalkanesulfinyl(C₁-C₆)alkyl, (C₄-C₈)cycloalkylalkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkanesulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkanesulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, di(C₁-C₈)alkylaminocarboxy(C₁-C₆)alkyl, phenyl, napthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, napthyl(C₁-C₃)alkyl, heteroaryl(C₁-C₃)alkyl, and bicyclic heteroaryl(C₁-C₃)alkyl; wherein the aromatic and heteroaromatic groups are optionally substituted with 1 to 3 groups independently selected from: fluoride, chloride, cyano, (C₁-C₃)alkyl, halo(C₁-C₃)alkyl, (C₁-C₃)alkoxy, halo(C₁-C₃)-alkoxy, (C₁-C₃)alkanesulfonyl, and (C₁-C₃)alkoxycarbonyl.

In a particular embodiment, R⁷ and R⁸ are both hydrogen and R⁵ and R⁶ are independently selected from: 1) hydrogen; (C₁-C₁₀)alkyl, (C₃-C₇)cycloalkyl(C₁-C₂)alkyl, (C₄-C₁₀)bicycloalkyl(C₁-C₂)alkyl, (C₈-C₁₂)tricycloalkyl(C₁-C₂)alkyl, saturated heterocyclyl(C₁-C₃)alkyl, (C₁-C₆)alkoxy(C₁-C₆)alkyl, (C₃-C₇)cycloalkoxy(C₁-C₃)alkyl, or (C₁-C₅)alkylthio(C₁-C₅)alkyl; wherein each of these groups are optionally substituted by 1 to 3 groups independently selected from: halogen, cyano, hydroxy, (C₁-C₂)alkyl, (C₁-C₂)alkoxy, (C₃-C₆)cycloalkyl, (C₃-C₆)cycloalkoxy, halo(C₁-C₂)alkyl, halo(C₁-C₂)alkoxy, halo(C₃-C₆)cycloalkyl, and halo(C₃-C₆)cycloalkoxy; and 2) phenyl(C₁-C₂)alkyl or heteroaryl(C₁-C₂)alkyl, each optionally substituted with 1 to 3 groups independently selected from: fluoride, chloride, cyano, (C₁-C₃)alkyl, halo(C₁-C₃)alkyl, (C₁-C₃)alkoxy, and halo(C₁-C₃)alkoxy;

In a more particular embodiment, R⁷ and R⁸ are both hydrogen, and R⁵ and R⁶ are each independently hydrogen, (C₃-C₇)cycloalkyl(C₁-C₂)alkyl, saturated heterocyclyl(C₁-C₃)alkyl, (C₃-C₇)cycloalkoxy(C₁-C₃)alkyl, or heteroaryl(C1-C2)alkyl, wherein each group is optionally and individually substituted with 1 to 3 groups selected from: fluoride, chloride, cyano, (C₁-C₃)alkyl, halo(C₁-C₃)alkyl, (C₁-C₃)alkoxy, or halo(C₁-C₃)alkoxy. In a further embodiment, either one of R⁵ or R⁶ is hydrogen. In a further embodiment, when one or more atoms of L are part of a saturated ring, R⁵ and R⁶ can both be hydrogen. In a more particular embodiment, R⁵ is cyclohexylmethyl or 3-(tetrahydropyranyl)methyl and R⁶ is H, or R⁵ is H and R⁶ is cyclohexylmethyl or 3-(tetrahydropyranyl)methyl.

G is —OH, —OR⁹, —NH₂, —NHR⁹, —NR⁹R¹⁰, —C(═NH)NH₂, —C(═NH)NHR⁹, —NHC(═NH)NH₂, or —NHC(═NH)NHR⁹.

R⁹ is a) (C₁-C₁₂)alkyl, (C₄-C₁₂)cycloalkylalkyl, halo(C₁-C₁₂)alkyl, halo(C₄-C₁₂)cycloalkylalkyl, (C₂-C₁₂)alkenyl, (C₅-C₁₂)cycloalkylalkenyl, halo(C₂-C₁₂)alkenyl, halo(C₅-C₁₂)cycloalkylalkenyl, (C₂-C₁₂)alkynyl, (C₅-C₁₂)cycloalkylalkynyl, halo(C₂-C₁₂)alkynyl, halo(C₅-C₁₂)cycloalkylalkynyl, (C₁-C₆)alkoxy(C₁-C₆)alkyl, halo(C₁-C₆)alkoxy(C₁-C₆)alkyl, (C₁-C₆)alkylthio(C₁-C₆)alkyl, halo(C₁-C₆)alkylthio(C₁-C₆)alkyl, (C₁-C₆)alkanesulfinyl(C₁-C₆)alkyl, halo(C₁-C₆)alkanesulfinyl(C₁-C₆)alkyl, (C₁-C₆)alkanesulfonyl(C₁-C₆)alkyl, halo(C₁-C₆)alkanesulfonyl(C₁-C₆)alkyl, aminocarbonyl(C₁-C₆)alkyl, (C₁-C₆)alkylaminocarbonyl(C₁-C₆)alkyl, di(C₁-C₆)alkylaminocarbonyl(C₁-C₆)alkyl, cyano(C₁-C₆)alkyl, carboxy(C₁-C₆)alkyl, (C₁-C₆)alkoxycarbonyl(C₁-C₆)alkyl, saturated heterocyclyl, or saturated heterocyclyl(C₁-C₆)alkyl; or b) phenyl, naphthyl, heteroaryl, phenyl(C₁-C₃)alkyl, naphthyl(C₁-C₃)alkyl, or heteroaryl(C₁-C₃)alkyl, each optionally and independently substituted by 1 to 3 groups selected from: 1) fluoride, chloride, bromide, iodide, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkylthio(C₁-C₆)alkyl, halo(C₄-C₈)cycloalkylalkylthio(C₁-C₆)alkyl, (C₁-C₈)alkanesulfinyl(C₁-C₆)alkyl, (C₃-C₈)cycloalkanesulfinyl(C₁-C₆)alkyl, (C₄-C₈)cycloalkylalkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkanesulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkanesulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl, (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl and di(C₁-C₈)alkylaminocarboxy(C₁-C₆)alkyl; and 2) phenyl, napthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, napthyl(C₁-C₃)alkyl, heteroaryl(C₁-C₃)alkyl, and bicyclic heteroaryl(C₁-C₃)alkyl, each optionally substituted with 1 to 3 groups independently selected from: fluoride, chloride, cyano, (C₁-C₃)alkyl, halo(C₁-C₃)alkyl, (C₁-C₃)alkoxy, halo(C₁-C₃)alkoxy, (C₁-C₃)alkanesulfonyl, and (C₁-C₃)-alkoxycarbonyl.

Alternatively, R⁹ taken together with one of R⁵, R⁶, R⁷ or R⁸ and their intervening atoms form a saturated 3-, 4-, 5-, 6-, or 7-membered “L-G ring” comprising 3 to 7 carbon atoms, and 1 or 2 heteroatoms selected from 0 or 1 nitrogen atoms, 0 or 1 oxygen atoms, and 0 or 1 sulfur atoms; said L-G ring being optionally substituted with 1 to 4 groups selected from: halogen, (C₁-C₈)alkyl, halo(C₁-C₈)alkyl, (C₃-C₈)cycloalkyl, (C₁-C₂)alkyl(C₃-C₆)cycloalkyl, halo(C₃-C₈)cycloalkyl, hydroxy(C₃-C₈)cycloalkyl, (C₃-C₈)cycloalkyl(C₁-C₃)alkyl, halo(C₃-C₈)cycloalkyl(C₁-C₃)alkyl, hydroxy(C₃-C₈)cycloalkyl(C₁-C₃)alkyl, (C₁-C₈)alkoxy, halo(C₁-C₈)alkoxy, (C₃-C₈)cycloalkoxy, halo(C₃-C₈)cycloalkoxy, hydroxy(C₃-C₈)cycloalkoxy, (C₁-C₈)alkoxy(C₁-C₃)alkyl, halo(C₁-C₈)alkoxy(C₁-C₃)alkyl, (C₃-C₈)cycloalkoxy(C₁-C₃)alkyl, halo(C₃-C₈)cycloalkoxy(C₁-C₃)alkyl, hydroxy(C₃-C₈)cycloalkoxy(C₁-C₃)alkyl, (C₃-C₈)cycloalkyl(C₁-C₃)alkoxy(C₁-C₃)alkyl, halo(C₃-C₈)cycloalkyl(C₁-C₃)alkoxy(C₁-C₃)alkyl, hydroxy(C₃-C₈)cycloalkyl(C₁-C₃)alkoxy(C₁-C₃)alkyl, (C₁-C₈)alkylthio, halo(C₁-C₈)alkylthio, (C₃-C₈)cycloalkylthio, halo(C₃-C₈)cycloalkylthio, hydroxy(C₃-C₈)cycloalkylthio, (C₃-C₈)cycloalkyl(C₁-C₃)alkylthio, halo(C₃-C₈)cycloalkyl(C₁-C₃)alkylthio, hydroxy(C₃-C₈)cycloalkyl(C₁-C₃)alkylthio, (C₁-C₈)alkylthio(C₁-C₃)alkyl, halo(C₁-C₈)alkylthio(C₁-C₃)alkyl, (C₃-C₈)cycloalkylthio(C₁-C₃)alkyl, halo(C₃-C₈)cycloalkylthio(C₁-C₃)alkyl, hydroxy(C₃-C₈)cycloalkylthio(C₁-C₃)alkyl, (C₃-C₈)cycloalkyl(C₁-C₃)alkylthio(C₁-C₃)alkyl, halo(C₃-C₈)cycloalkyl(C₁-C₃)alkylthio(C₁-C₃)alkyl, hydroxy(C₃-C₈)cycloalkyl(C₁-C₃)alkylthio(C₁-C₃)alkyl and oxo.

In a more particular embodiment, R⁹ is 1) hydrogen, (C₁-C₆)alkyl, halo(C₁-C₆)alkyl, (C₄-C₁₀)cycloalkylalkyl, (C₁-C₅)alkoxy(C₁-C₅)alkyl, aminocarbonyl(C₁-C₅)alkyl, (C₁-C₆)alkylaminocarbonyl(C₁-C₆)alkyl, or di(C₁-C₆)alkylaminocarbonyl(C₁-C₆)alkyl; or 2) phenyl(C₁-C₂)alkyl, optionally substituted with 1 to 3 groups independently selected from: fluoride, chloride, cyano, (C₁-C₃)alkyl, halo(C₁-C₃)alkyl, (C₁-C₃)alkoxy, and halo(C₁-C₃)alkoxy.

In a further particular embodiment, R⁹ is: hydrogen, (C₁-C₃)alkyl, halo(C₁-C₃)alkyl, (C₄-C₈)cycloalkylalkyl, or (C₁-C₃)alkoxy(C₁-C₃)alkyl. In an additional particular embodiment, R⁹ is hydrogen or methyl.

R¹⁰ is (C₁-C₆)alkyl or halo(C₁-C₆)alkyl.

The variable n is an integer from 0 to 5. Particularly, n is an integer from 1 to 4. More particularly, n is an integer from 0 to 3.

Each R¹¹ is independently selected from the groups consisting of: 1) fluoride, chloride, bromide, iodide, cyano, nitro, amino, hydroxy, carboxy, (C₁-C₈)alkyl, (C₃-C₈)cycloalkyl, (C₁-C₃)alkyl(C₃-C₈)cycloalkyl, di(C₁-C₃)alkyl(C₃-C₈)cycloalkyl, (C₄-C₈)cycloalkylalkyl, (C₂-C₆)alkenyl, (C₅-C₈)cycloalkenyl, (C₅-C₈)cycloalkylalkenyl, (C₂-C₈)alkynyl, (C₃-C₈)cycloalkyl(C₂-C₄)alkynyl, halo(C₁-C₈)alkyl, halo(C₃-C₆)cycloalkyl, halo(C₄-C₈)cycloalkylalkyl, (C₁-C₃)alkyl(C₄-C₈)cycloalkylalkyl, di(C₁-C₃)alkyl(C₄-C₈)cycloalkylalkyl, halo(C₂-C₈)alkenyl, halo(C₅-C₈)cycloalkenyl, halo(C₆-C₈)cycloalkenylalkyl, halo(C₃-C₈)alkynyl, halo(C₅-C₈)cycloalkylalkynyl, (C₁-C₈)alkoxy, (C₃-C₈)cycloalkoxy, (C₄-C₈)cycloalkylalkoxy, (C₁-C₃)alkyl(C₃-C₈)cycloalkoxy, (C₁-C₃)alkyl(C₄-C₈)cycloalkylalkoxy, di(C₁-C₃)alkyl(C₃-C₈)-cycloalkoxy, di(C₁-C₃)alkyl(C₄-C₈)cycloalkylalkoxy, halo(C₁-C₆)alkoxy, halo(C₃-C₆)cycloalkoxy, halo(C₄-C₈)cycloalkylalkoxy, (C₁-C₈)alkylthio, (C₃-C₈)cycloalkylthio, (C₄-C₈)cycloalkylalkylthio, (C₁-C₃)alkyl(C₃-C₈)cycloalkylthio, (C₁-C₃)alkyl(C₄-C₈)cycloalkylalkylthio, di(C₁-C₃)alkyl(C₃-C₈)cycloalkylthio, di(C₁-C₃)alkyl(C₄-C₈)cycloalkylalkylthio, halo(C₁-C₈)alkylthio, halo(C₃-C₈)cycloalkylthio, halo(C₄-C₈)cycloalkylalkylthio, (C₁-C₈)alkanesulfinyl, (C₃-C₈)cycloalkanesulfinyl, (C₄-C₈)cycloalkylalkanesulfinyl, (C₁-C₃)alkyl(C₃-C₈)cycloalkanesulfinyl, (C₁-C₃)alkyl(C₄-C₈)cycloalkylalkanesulfinyl, di(C₁-C₃)alkyl(C₃-C₈)cycloalkanesulfinyl, di(C₁-C₃)alkyl(C₄-C₈)cycloalkylalkanesulfinyl, halo(C₁-C₈)alkanesulfinyl, halo(C₃-C₈)cycloalkanesulfinyl, halo(C₄-C₈)cycloalkylalkanesulfinyl, (C₁-C₈)alkanesulfonyl, (C₃-C₈)cycloalkanesulfonyl, (C₄-C₈)cycloalkylalkanesulfonyl, (C₁-C₃)alkyl(C₃-C₈)cycloalkanesulfonyl, (C₁-C₃)alkyl(C₄-C₈)cycloalkylalkanesulfonyl, di(C₁-C₃)alkyl(C₃-C₈)cycloalkanesulfonyl, di(C₁-C₃)alkyl(C₄-C₈)cycloalkylalkanesulfonyl, halo(C₁-C₈)alkanesulfonyl, halo(C₃-C₈)cycloalkanesulfonyl, halo(C₄-C₈)cycloalkylalkanesulfonyl, (C₁-C₈)alkylamino, di(C₁-C₈)alkylamino, (C₁-C₆)alkoxy(C₁-C₆)alkoxy, halo(C₁-C₆)alkoxy(C₁-C₆)alkoxy, (C₁-C₈)alkoxycarbonyl, aminocarbonyl, (C₁-C₈)alkylaminocarbonyl, di(C₁-C₈)alkylaminocarbonyl, piperidino, pyrrolidino, cyano(C₁-C₆)alkyl, hydroxy(C₁-C₆)alkyl, carboxy(C₁-C₆)alkyl, (C₁-C₈)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkylthio(C₁-C₆)alkyl, halo(C₄-C₈)cycloalkylalkylthio(C₁-C₆)alkyl, (C₁-C₈)alkanesulfinyl(C₁-C₆)alkyl, (C₃-C₈)cycloalkanesulfinyl(C₁-C₆)alkyl, (C₄-C₈)cycloalkylalkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkanesulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkanesulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl, (C₁-C₈)acylamino(C₁-C₆)alkyl, piperidino(C₁-C₆)alkyl, pyrrolidino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, or di(C₁-C₈)alkylaminocarboxy(C₁-C₆)alkyl; and 2) phenyl, napthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, bicyclic heteroaryl(C₁-C₃)alkyl, phenyl(C₁-C₃)alkoxy, naphthyl(C₁-C₃)alkoxy, heteroaryl(C₁-C₃)alkoxy, and bicyclic heteroaryl(C₁-C₃)alkoxy, each optionally substituted with 1 to 5 groups independently selected from the group consisting of: fluoride, chloride, cyano, (C₁-C₆)alkyl, halo(C₁-C₆)alkyl, (C₁-C₆)alkoxy, halo(C₁-C₆)alkoxy, (C₁-C₆)alkanesulfonyl, (C₁-C₆)alkoxycarbonyl and aminocarbonyl.

More particularly, each R¹¹ is independently selected from the groups consisting of: 1) fluoride, chloride, bromide, cyano, nitro, (C₁-C₆)alkyl, (C₃-C₆)cycloalkyl, (C₄-C₇)cycloalkylalkyl, (C₂-C₆)alkenyl, (C₅-C₆)cycloalkenyl, (C₅-C₈)cycloalkylalkenyl, (C₂-C₆)alkynyl, (C₃-C₆)cycloalkylethynyl, halo(C₁-C₆)alkyl, halo(C₃-C₆)cycloalkyl, halo(C₄-C₇)cycloalkylalkyl, halo(C₂-C₆)alkenyl, halo(C₃-C₆)alkynyl, halo(C₃-C₆)cycloalkylethynyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₃-C₆)alkenyloxy, and (C₁-C₆)alkanesulfonyl; or 2) phenyl, heteroaryl, phenoxy, heteroaryloxy, phenylthio, heteroarylthio, benzyl, heteroarylmethyl, benzyloxy, and heteroaryloxy, each optionally substituted with 1 to 3 groups independently selected from: fluoride, chloride, cyano, (C₁-C₃)alkyl, halo(C₁-C₃)alkyl, (C₁-C₃)alkoxy, halo(C₁-C₃)alkoxy, and aminocarbonyl.

Even more particularly, each R¹¹ is independently selected from: fluoride, (C₁-C₃) alkyl, halo(C₁-C₃)alkyl, and oxo, and wherein the phenyl and heteroaryl are optionally and independently substituted with 1 to 3 groups selected from: halogen, nitro, cyano, (C₁-C₆)alkyl, halo(C₁-C₆)alkyl, (C₁-C₆)alkoxy, halo(C₁-C₆)alkoxy, phenyl and heteroaryl.

A first particular embodiment of the invention is a compound according to Formulae IIa, IIIa, IVa, Va, VIa, VIIa, VIIIa, IXa or Xa:

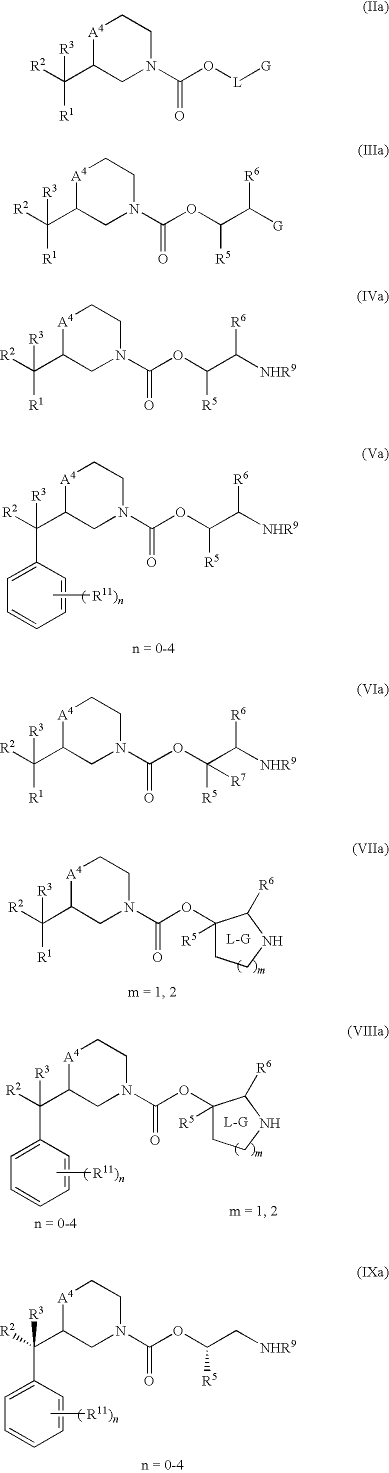

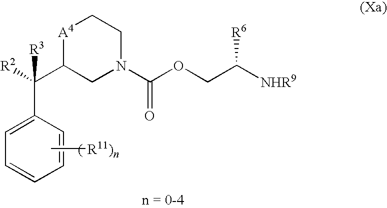

or an enantiomer, diastereomer, or pharmaceutically acceptable salt thereof. Values and particular values for each variable in Formulae Ila, IIIa, IVa, Va, VIa, VIIa, VIIIa, IXa, and Xa are as described for Formula I.

More particularly, in Formula IIa:

L is: 1) a linear (C₂-C₄)alkyl chain when G is —OH, —OR⁹, —NH₂, —NHR⁹, —NR⁹R¹⁰, —NHC(═NH)NH₂, or —NHC(═NH)NHR⁹; or 2) a linear (C₁-C₃)alkyl chain when G is —C(═NH)NH₂ or —C(═NH)NHR⁹; and L is substituted by 1-4 groups independently selected from R⁵, R⁶, R⁷, and R⁸.

Each R⁵, R⁶, R⁷, and R⁸ is independently selected from: 1) hydrogen; 2) (C₁-C₁₂)alkyl, (C₃-C₈)cycloalkyl, (C₃-C₈)cycloalkyl(C₁-C₃)alkyl, (C₂-C₁₂)alkenyl, (C₅-C₈)cycloalkyl(C₁-C₃)alkenyl, (C₂-C₁₂)alkynyl, (C₃-C₈)cycloalkyl(C₁-C₃)alkynyl, (C₄-C₁₂)bicycloalkyl(C₁-C₃)alkyl, (C₈-C₁₄)tricycloalkyl(C₁-C₃)alkyl, (C₁-C₆)alkoxy(C₁-C₆)alkyl, (C₃-C₈)cycloalkoxy(C₁-C₃)alkyl, (C₁-C₆)alkylthio(C₁-C₆)alkyl, (C₃-C₈)cycloalkylthio(C₁-C₃)alkyl, saturated heterocyclyl, and saturated heterocyclyl(C₁-C₃)alkyl; wherein each R⁵, R⁶, R⁷ and R⁸ is optionally and independently substituted by a group selected from: halogen, cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, halo(C₃-C₆)cycloalkoxy; and divalent sulfur atoms are optionally oxidized to sulfoxide or sulfone; and 3) phenyl, naphthyl, heteroaryl, phenyl(C₁-C₃)alkyl, naphthyl(C₁-C₃)alkyl, and heteroaryl(C₁-C₃)alkyl; each optionally substituted with 1 to 3 groups independently selected from: fluoride, chloride, bromide, iodide, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkythio(C₁-C₆)alkyl, halo(C₄-C₈)-cycloalkylalkylthio(C₁-C₆)alkyl, (C₁-C₈)alkanesulfinyl(C₁-C₆)alkyl, (C₃-C₈)cycloalkanesulfinyl(C₁-C₆)alkyl, (C₄-C₈)cycloalkylalkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkanesulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkanesulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, di(C₁-C₈)alkylaminocarboxy(C₁-C₆)alkyl, phenyl, napthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, napthyl(C₁-C₃)alkyl, heteroaryl(C₁-C₃)alkyl, and bicyclic heteroaryl(C₁-C₃)alkyl; wherein the aromatic and heteroaromatic groups are optionally substituted with 1 to 3 groups independently selected from: fluoride, chloride, cyano, (C₁-C₃)alkyl, halo(C₁-C₃)alkyl, (C₁-C₃)alkoxy, halo(C₁-C₃)-alkoxy, (C₁-C₃)alkanesulfonyl, and (C₁-C₃)alkoxycarbonyl.

R⁹ is: a) (C₁-C₁₂)alkyl, (C₄-C₁₂)cycloalkylalkyl, halo(C₁-C₁₂)alkyl, halo(C₄-C₁₂)cycloalkylalkyl, (C₂-C₁₂)alkenyl, (C₅-C₁₂)cycloalkylalkenyl, halo(C₂-C₁₂)alkenyl, halo(C₅-C₁₂)cycloalkylalkenyl, (C₂-C₁₂)alkynyl, (C₅-C₁₂)cycloalkylalkynyl, halo(C₂-C₁₂)alkynyl, halo(C₅-C₁₂)cycloalkylalkynyl, (C₁-C₆)alkoxy(C₁-C₆)alkyl, halo(C₁-C₆)alkoxy(C₁-C₆)alkyl, (C₁-C₆)alkylthio(C₁-C₆)alkyl, halo(C₁-C₆)alkylthio(C₁-C₆)alkyl, (C₁-C₆)alkanesulfinyl(C₁-C₆)alkyl, halo(C₁-C₆)alkanesulfinyl(C₁-C₆)alkyl, (C₁-C₆)alkanesulfonyl(C₁-C₆)alkyl, halo(C₁-C₆)alkanesulfonyl(C₁-C₆)alkyl, aminocarbonyl(C₁-C₆)alkyl, (C₁-C₆)alkylaminocarbonyl(C₁-C₆)alkyl, di(C₁-C₆)alkylaminocarbonyl(C₁-C₆)alkyl, cyano(C₁-C₆)alkyl, carboxy(C₁-C₆)alkyl, (C₁-C₆)alkoxycarbonyl(C₁-C₆)alkyl, saturated heterocyclyl, or saturated heterocyclyl(C₁-C₆)alkyl; or b) phenyl, naphthyl, heteroaryl, phenyl(C₁-C₃)alkyl, naphthyl(C₁-C₃)alkyl, or heteroaryl(C₁-C₃)alkyl, each optionally and independently substituted by 1 to 3 groups selected from: 1) fluoride, chloride, bromide, iodide, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkylthio(C₁-C₆)alkyl, halo(C₄-C₈)cycloalkylalkylthio(C₁-C₆)alkyl, (C₁-C₈)alkanesulfinyl(C₁-C₆)alkyl, (C₃-C₈)cycloalkanesulfinyl(C₁-C₆)alkyl, (C₄-C₈)cycloalkylalkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkanesulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkanesulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl, (C₁-C₈)acylamino(C₁-C₆)alkyl, (C₁-C₈)alkoxycarbonylamino, C₁-C₈)alkoxycarbonylamino(C₁-C₆)alkyl, aminocarboxy(C₁-C₆)alkyl, (C₁-C₈)alkylaminocarboxy(C₁-C₆)alkyl and di(C₁-C₈)alkylaminocarboxy(C₁-C₆)alkyl; and 2) phenyl, napthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, napthyl(C₁-C₃)alkyl, heteroaryl(C₁-C₃)alkyl, and bicyclic heteroaryl(C₁-C₃)alkyl, each optionally substituted with 1 to 3 groups independently selected from: fluoride, chloride, cyano, (C₁-C₃)alkyl, halo(C₁-C₃)alkyl, (C₁-C₃)alkoxy, halo(C₁-C₃)alkoxy, (C₁-C₃)alkanesulfonyl, and (C₁-C₃)-alkoxycarbonyl. The other variables are as described for Formula Ia.

A second particular embodiment of the present invention is a compound according to Formulae IIa, IIIa, IVa, Va, IXa or Xa wherein:

R¹, in Formulae IIa, IIIa or IVa, is: a) (C₁-C₉)alkyl, (C₃-C₇)cycloalkyl, (C₄-C₉)cycloalkylalkyl, halo(C₁-C₉)alkyl, halo(C₃-C₇)cycloalkyl, halo(C₄-C₉)cycloalkylalkyl, or saturated heterocyclyl each optionally substituted with 1 to 3 groups independently selected from: fluoride, (C₁-C₃)alkyl, halo(C₁-C₃)alkyl, and oxo; or b) phenyl, napthyl, heteroaryl or bicyclic heteroaryl, each substituted with n groups represented by R¹¹, wherein n is an integer from 0 to 3, and wherein each R¹¹ is independently selected from the groups consisting of: 1) fluoride, chloride, bromide, cyano, nitro, (C₁-C₆)alkyl, (C₃-C₆)cycloalkyl, (C₄-C₇)cycloalkylalkyl, (C₂-C₆)alkenyl, (C₅-C₆)cycloalkenyl, (C₅-C₈)cycloalkylalkenyl, (C₂-C₆)alkynyl, (C₃-C₆)cycloalkylethynyl, halo(C₁-C₆)alkyl, halo(C₃-C₆)cycloalkyl, halo(C₄-C₇)cycloalkylalkyl, halo(C₂-C₆)alkenyl, halo(C₃-C₆)alkynyl, halo(C₃-C₆)cycloalkylethynyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₃-C₆)alkenyloxy, or (C₁-C₆)alkanesulfonyl; and 2) phenyl, heteroaryl, phenoxy, heteroaryloxy, phenylthio, heteroarylthio, benzyl, heteroarylmethyl, benzyloxy, and heteroaryloxy, each optionally substituted with 1 to 3 groups independently selected from: fluoride, chloride, cyano, (C₁-C₃)alkyl, halo(C₁-C₃)alkyl, (C₁-C₃)alkoxy, halo(C₁-C₃)alkoxy, or aminocarbonyl.

R² is (C₁-C₈)alkyl, (C₄-C₈)cycloalkylalkyl, fluoro(C₁-C₈)alkyl, fluoro(C₄-C₈)cycloalkylalkyl, (C₁-C₈)alkoxy, (C₄-C₈)cycloalkylalkoxy, fluoro(C₁-C₈)alkoxy, hydroxy(C₁-C₈)alkyl, (C₁-C₅)alkoxy(C₁-C₅)alkyl, halo(C₁-C₅)alkylamino(C₁-C₅)alkyl, (C₁-C₅)alkoxy(C₁-C₅)hydroxyalkyl, (C₃-C₄)cycloalkoxy(C₁-C₅)alkyl, fluoro(C₁-C₅)alkoxy(C₁-C₅)alkyl, fluoro(C₃-C₄)cycloalkoxy(C₁-C₅)alkyl, (C₁-C₅)alkylthio(C₁-C₅)alkyl, (C₁-C₅)alkoxy(C₁-C₅)alkoxy, hydroxy(C₁-C₈)alkoxy, (C₃-C₄)cycloalkoxy(C₁-C₅)alkoxy, fluoro(C₁-C₅)alkoxy(C₁-C₅)alkoxy, fluoro(C₃-C₄)cycloalkoxy(C₁-C₅)alkoxy, (C₁-C₃)alkoxy(C₁-C₃)alkoxy(C₁-C₃)alkyl, fluoro(C₁-C₃)-alkoxy(C₁-C₃)alkoxy(C₁-C₃)alkyl, aminocarbonylamino(C₁-C₈)alkyl, aminocarbonylamino(C₁-C₈)alkoxy, (C₁-C₅)alkanoylamino(C₁-C₅)alkyl, (C₁-C₅)alkanoylamino(C₁-C₅)alkoxy, fluoro(C₁-C₅)alkanoylamino(C₁-C₅)alkyl, fluoro(C₁-C₅)alkanoylamino(C₁-C₅)alkoxy, (C₁-C₃)alkoxy(C₁-C₅)alkanoylamino(C₁-C₅)alkyl, (C₁-C₃)alkoxy(C₁-C₅)alkanoylamino(C₁-C₅)alkoxy, (C₃-C₄)cycloalkylcarbonylamino(C₁-C₅)alkyl, (C₃-C₄)cycloalkylcarbonylamino(C₁-C₅)alkoxy, aminosulfonylamino(C₁-C₈)alkyl, aminosulfonylamino(C₁-C₈)alkoxy, (C₁-C₅)alkanesulfonylamino(C₁-C₅)alkyl, (C₁-C₅)alkanesulfonylamino(C₁-C₅)alkoxy, formylamino(C₁-C₅)alkyl, formylamino(C₁-C₅)alkoxy, (C₁-C₅)alkoxycarbonylamino(C₁-C₅)alkyl, (C₁-C₅)alkoxycarbonylamino(C₁-C₅)alkoxy, (C₁-C₅)alkylaminocarbonylamino(C₁-C₅)alkyl, (C₁-C₅)alkylaminocarbonylamino(C₁-C₅)alkyl, di(C₁-C₅)alkylaminocarbonylamino(C₁-C₅)alkoxy, aminocarbonyl(C₁-C₅)alkyl, aminocarbonyl(C₁-C₅)alkoxy, (Cₗ-C₅)alkylaminocarbonyl(C₁-C₅)alkyl, (C₁-C₅)alkylaminocarbonyl(C₁-C₅)alkoxy, aminocarboxy(C₁-C₅)alkyl, aminocarboxy(C₁-C₅)alkoxy, (C₁-C₅)alkylaminocarboxy(C₁-C₅)alkyl, (C₁-C₅)alkylaminocarboxy(C₁-C₅)alkoxy, (C₁-C₈)alkoxycarbonylamino, (C₁-C₈)alkylaminocarbonylamino, (C₁-C₈)alkanoylamino, fluoro(C₁-C₈)alkoxycarbonylamino, fluoro(C₁-C₈)alkylaminocarbonylamino, (C₁-C₈)oxoalkyl, or fluoro(C₁-C₈)alkanoylamino.

R³ is —H, halogen, —OH, (C₁-C₄)alkanoylamino, or (C₁-C₃)alkoxy, with the proviso that when R³ is —OH or halogen, then R² is not (C₁-C₈)alkoxy, (C₄-C₈)cycloalkylalkoxy, fluoro(C₁-C₈)alkoxy, (C₁-C₅)alkoxy(C₁-C₅)alkoxy, hydroxy(C₁-C₈)alkoxy, (C₃-C₄)cycloalkoxy(C₁-C₅)alkoxy, fluoro(C₁-C₅)alkoxy(C₁-C₅)alkoxy, fluoro(C₃-C₄)cycloalkoxy(C₁-C₅)alkoxy, aminocarbonylamino(C₁-C₈)alkoxy, (C₁-C₅)alkanoylamino(C₁-C₅)alkoxy, fluoro(C₁-C₅)alkanoylamino(C₁-C₅)alkoxy, (C₁-C₃)alkoxy(C₁-C₅)alkanoylamino(C₁-C₅)alkoxy, (C₃-C₄)cycloalkylcarbonylamino(C₁-C₅)alkoxy, aminosulfonylamino(C₁-C₈)alkoxy, (C₁-C₅)alkanesulfonylamino(C₁-C₅)alkoxy, formylamino(C₁-C₅)alkoxy, (C₁-C₅)alkoxycarbonylamino(C₁-C₅)alkoxy, di(C₁-C₅)alkylaminocarbonylamino(C₁-C₅)alkoxy, aminocarbonyl(C₁-C₅)alkoxy, (C₁-C₅)alkylaminocarbonyl(C₁-C₅)alkoxy, aminocarboxy(C₁-C₅)alkoxy, (C₁-C₅)alkylaminocarboxy(C₁-C₅)alkoxy, (C₁-C₈)alkoxycarbonylamino, (C₁-C₈)alkylaminocarbonylamino, (C₁-C₈)alkanoylamino, fluoro(C₁-C₈)alkoxycarbonylamino, fluoro(C₁-C₈)alkylaminocarbonylamino, or fluoro(C₁-C₈)alkanoylamino.

A⁴ is CH₂, O, CH₂CH₂ or CH₂O.

R⁵ and R⁶, where present, are independently selected from: 1) hydrogen; (C₁-C₁₀)alkyl, (C₃-C₇)cycloalkyl(C₁-C₂)alkyl, (C₄-C₁₀)bicycloalkyl(C₁-C₂)alkyl, (C₈-C₁₂)tricycloalkyl(C₁-C₂)alkyl, saturated heterocyclyl(C₁-C₃)alkyl, (C₁-C₆)alkoxy(C₁-C₆)alkyl, (C₃-C₇)cycloalkoxy(C₁-C₃)alkyl, or (C₁-C₅)alkylthio(C₁-C₅)alkyl; each of these groups are optionally substituted by 1 to 3 groups independently selected from: halogen, cyano, hydroxy, (C₁-C₂)alkyl, (C₁-C₂)alkoxy, (C₃-C₆)cycloalkyl, (C₃-C₆)cycloalkoxy, halo(C₁-C₂)alkyl, halo(C₁-C₂)alkoxy, halo(C₃-C₆)cycloalkyl, and halo(C₃-C₆)cycloalkoxy; and 2) phenyl(C₁-C₂)alkyl or heteroaryl(C₁-C₂)alkyl, each optionally substituted with 1 to 3 groups independently selected from: fluoride, chloride, cyano, (C₁-C₃)alkyl, halo(C₁-C₃)alkyl, (C₁-C₃)alkoxy, and halo(C₁-C₃)alkoxy.

R⁹ is: 1) hydrogen, (C₁-C₆)alkyl, halo(C₁-C₆)alkyl, (C₄-C₁₀)cycloalkylalkyl, (C₁-C₅)alkoxy(C₁-C₅)alkyl, aminocarbonyl(C₁-C₅)alkyl, (C₁-C₆)alkylaminocarbonyl(C₁-C₆)alkyl, or di(C₁-C₆)alkylaminocarbonyl(C₁-C₆)alkyl; or 2) phenyl(C₁-C₂)alkyl, optionally substituted with 1 to 3 groups independently selected from: fluoride, chloride, cyano, (C₁-C₃)alkyl, halo(C₁-C₃)alkyl, (C₁-C₃)alkoxy, and halo(C₁-C₃)alkoxy.

Values and particular values for L are as described in the first embodiment; and G, in Formulae IIa and IIIa, are as described for Formula Ia.

A third embodiment of the invention is a compound according to Formulae IIa, IIIa, IVa, Va, IXa, or Xa wherein:

R¹, in Formulae IIa, IIIa, and IVa, is: a saturated heterocycle, phenyl, or heteroaryl; wherein the saturated heterocycle is substituted with n groups, represented by R¹¹, wherein n is an integer from 0 to 3, and wherein each R¹¹ is independently selected from: fluoride, (C₁-C₃) alkyl, halo(C₁-C₃)alkyl, and oxo, and wherein the phenyl and heteroaryl are optionally and independently substituted with 1 to 3 groups selected from: halogen, nitro, cyano, (C₁-C₆)alkyl, halo(C₁-C₆)alkyl, (C₁-C₆)alkoxy, halo(C₁-C₆)alkoxy, phenyl and heteroaryl.

R² is: (C₁-C₅)alkoxy(C₁-C₅)alkyl, (C₁-C₅)alkoxy(C₁-C₅)hydroxyalkyl, (C₃-C₄)cycloalkoxy(C₁-C₅)alkyl, fluoro(C₁-C₅)alkoxy(C₁-C₅)alkyl, (C₁-C₅)alkoxy, (C₁-C₅)alkoxy, fluoro(C₁-C₅)alkoxy(C₁-C₅)alkoxy, (C₁-C₃)alkoxy(C₁-C₃)alkoxy(C₁-C₃)alkyl, fluoro(C₁-C₃)alkoxy(C₁-C₃)alkoxy(C₁-C₃)alkyl, (C₁-C₅)alkanoylamino(C₁-C₅)alkyl, (C₁-C₅)alkanoylamino(C₁-C₅)alkoxy, fluoro(C₁-C₅)alkanoylamino(C₁-C₅)alkyl, fluoro(C₁-C₅)alkanoylamino(C₁-C₅)alkoxy, (C₁-C₃)alkoxy(C₁-C₅)alkanoylamino(C₁-C₅)alkyl, (C₁-C₃)alkoxy(C₁-C₅)alkanoylamino(C₁-C₅)alkoxy, (C₁-C₅)alkoxycarbonylamino(C₁-C₅)alkyl, (C₁-C₅)alkoxycarbonylamino(C₁-C₅)alkoxy, (C₁-C₅)alkylaminocarbonylamino(C₁-C₅)alkyl, di(C₁-C₅)alkylaminocarbonylamino(C₁-C₅)alkoxy, (C₁-C₈)alkylaminocarbonylamino, fluoro(C₁-C₈)alkoxycarbonylamino, or (C₁-C₈)oxoalkyl.

R³ is hydrogen, fluoride, chloride, —OH or (C₁-C₃)alkoxy, provided that when R³ is —OH, —F, or —Cl, then R² is not (C₁-C₅)alkoxy(C₁-C₅)alkoxy, fluoro(C₁-C₅)alkoxy(C₁-C₅)alkoxy, (C₁-C₅)alkanoylamino(C₁-C₅)alkoxy, fluoro(C₁-C₅)alkanoylamino(C₁-C₅)alkoxy, (C₁-C₃)alkoxy(C₁-C₅)alkanoylamino(C₁-C₅)alkoxy, (C₁-C₅)alkoxycarbonylamino(C₁-C₅)alkoxy, di(C₁-C₅)alkylaminocarbonylamino(C₁-C₅)alkoxy, (C₁-C₈)alkylaminocarbonylamino, or fluoro(C₁-C₈)alkoxycarbonylamino.

A⁴ is CH₂ or O.

R⁵ and R⁶, where present, are each independently hydrogen, (C₃-C₇)cycloalkyl(C₁-C₂)alkyl, saturated heterocyclyl(C₁-C₃)alkyl, (C₃-C₇)cycloalkoxy(C₁-C₃)alkyl, or heteroaryl(C1-C2)alkyl, wherein each group is optionally and individually substituted with 1 to 3 groups selected from: fluoride, chloride, cyano, (C₁-C₃)alkyl, halo(C₁-C₃)alkyl, (C₁-C₃)alkoxy, or halo(C₁-C₃)alkoxy.

R⁹ is: hydrogen, (C₁-C₃)alkyl, halo(C₁-C₃)alkyl, (C₄-C₈)cycloalkylalkyl, or (C₁-C₃)alkoxy(C₁-C₃)alkyl.

Values and particular values for L are as described in the first embodiment; and G, in Formulae IIa and IIIa, are as described for Formula Ia.

A fourth particular embodiment of the present invention is a compound according to Formulae Ia, IIa, IIIa, IVa, Va, IXa, or Xa wherein R¹, in Formulae IIa, IIIa, or IVa, is phenyl optionally substituted with 1 to 3 substituents independently selected from fluoride, chloride and methyl; R² is 4-methoxybutyl, 3-(methoxycarbonylamino)propyl or 2-(methoxycarbonylamino)ethoxy; R³ is H or OH; provided that when R² is 2-(methoxycarbonylamino)ethoxy R³ is not —OH; A⁴ is CH₂ or O; R⁵ is cyclohexylmethyl or 3-(tetrahydropyranyl)methyl and R⁶ is H, or R⁵ is H and R⁶ is cyclohexylmethyl or 3-(tetrahydropyranyl)methyl; values and particular values for L are as described in the first embodiment; and G, in Formulae IIa and IIIa, are as described for Formula Ia.

More particularly, in the first, second, third, and fourth embodiments, is a compound wherein one of R⁵ or R⁶ is hydrogen, but not both, and the other variables are as described above.

Even more particularly, in the first, second, third and fourth embodiments, is a compound wherein one of R⁵ or R⁶ is hydrogen, but not both, and R² is 4-methoxybutyl, 3-(methoxycarbonylamino)propyl, or 2-(methoxycarbonylamino)ethoxy, and the other variables are as described above.

A fifth embodiment of the invention is a compound according to Formulae VIa, VIIa, or VIIIa, wherein R⁷, in Formula VIa, is taken together with R⁹, in Formula VIa, and their intervening atoms form a saturated 3-, 4-, 5-, 6-, or 7-membered “L-G ring” comprising 3 to 7 carbon atoms, and 1 or 2 hetero atoms selected from 1 nitrogen atom, 0 or 1 oxygen atoms, and 0 or 1 sulfur atoms; said L-G ring being optionally substituted with 1 to 4 groups selected from: halogen, (C₁-C₈)alkyl, halo(C₁-C₈)alkyl, (C₃-C₈)cycloalkyl, (C₁-C₂)alkyl(C₃-C₆)cycloalkyl, halo(C₃-C₈)cycloalkyl, hydroxy(C₃-C₈)cycloalkyl, (C₃-C₈)cycloalkyl(C₁-C₃)alkyl, halo(C₃-C₈)cycloalkyl(C₁-C₃)alkyl, hydroxy(C₃-C₈)cycloalkyl(C₁-C₃)alkyl, (C₁-C₈)alkoxy, halo(C₁-C₈)alkoxy, (C₃-C₈)cycloalkoxy, halo(C₃-C₈)cycloalkoxy, hydroxy(C₃-C₈)cycloalkoxy, (C₁-C₈)alkoxy(C₁-C₃)alkyl, halo(C₁-C₈)alkoxy(C₁-C₃)alkyl, (C₃-C₈)cycloalkoxy(C₁-C₃)alkyl, halo(C₃-C₈)cycloalkoxy(C₁-C₃)alkyl, hydroxy(C₃-C₈)cycloalkoxy(C₁-C₃)alkyl, (C₃-C₈)cycloalkyl(C₁-C₃)alkoxy(C₁-C₃)alkyl, halo(C₃-C₈)cycloalkyl(C₁-C₃)alkoxy(C₁-C₃)alkyl, hydroxy(C₃-C₈)cycloalkyl(C₁-C₃)alkoxy(C₁-C₃)alkyl, (C₁-C₈)alkylthio, halo(C₁-C₈)alkylthio, (C₃-C₈)cycloalkylthio, halo(C₃-C₈)cycloalkylthio, hydroxy(C₃-C₈)cycloalkylthio, (C₃-C₈)cycloalkyl(C₁-C₃)alkylthio, halo(C₃-C₈)cycloalkyl(C₁-C₃)alkylthio, hydroxy(C₃-C₈)cycloalkyl(C₁-C₃)alkylthio, (C₁-C₈)alkylthio(C₁-C₃)alkyl, halo(C₁-C₈)alkylthio(C₁-C₃)alkyl, (C₃-C₈)cycloalkylthio(C₁-C₃)alkyl, halo(C₃-C₈)cycloalkylthio(C₁-C₃)alkyl, hydroxy(C₃-C₈)cycloalkylthio(C₁-C₃)alkyl, (C₃-C₈)cycloalkyl(C₁-C₃)alkylthio(C₁-C₃)alkyl, halo(C₃-C₈)cycloalkyl(C₁-C₃)alkylthio(C₁-C₃)alkyl, hydroxy(C₃-C₈)cycloalkyl(C₁-C₃)alkylthio(C₁-C₃)alkyl and oxo.

Values and particular values of the remainder of variables are as described for Formula Ia.

A sixth embodiment of the invention is a compound according to Formulae VIa, VIIa, or VIIIa, wherein:

R¹, in Formulae VIa or VIIa, is 1) (C₁-C₉)alkyl, (C₃-C₇)cycloalkyl, (C₄-C₉)cycloalkylalkyl, halo(C₁-C₉)alkyl, halo(C₃-C₇)cycloalkyl, halo(C₄-C₉)cycloalkylalkyl, or saturated heterocyclyl, each optionally substituted with 1 to 3 groups independently selected from: fluoride, (C₁-C₃)alkyl, halo(C₁-C₃)alkyl, and oxo; or 2) phenyl, napthyl, heteroaryl, or bicyclic heteroaryl each optionally substituted with 1 to 3 groups independently selected from: a) fluoride, chloride, bromide, cyano, nitro, (C₁-C₆)alkyl, (C₃-C₆)cycloalkyl, (C₄-C₇)cycloalkylalkyl, (C₂-C₆)alkenyl, (C₅-C₆)cycloalkenyl, (C₅-C₈)cycloalkylalkenyl, (C₂-C₆)alkynyl, (C₃-C₆)cycloalkylethynyl, halo(C₁-C₆)alkyl, halo(C₃-C₆)cycloalkyl, halo(C₄-C₇)-cycloalkylalkyl, halo(C₂-C₆)alkenyl, halo(C₃-C₆)alkynyl, halo(C₃-C₆)cycloalkylethynyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₃-C₆)alkenyloxy, or (C₁-C₆)alkanesulfonyl; and b) phenyl, heteroaryl, phenoxy, heteroaryloxy, phenylthio, heteroarylthio, benzyl, heteroarylmethyl, benzyloxy, or heteroaryloxy, each optionally substituted with 1 to 3 groups independently selected from: fluoride, chloride, cyano, (C₁-C₃)alkyl, halo(C₁-C₃)alkyl, (C₁-C₃)alkoxy, halo(C₁-C₃)alkoxy, and aminocarbonyl.

R² is (C₁-C₈)alkyl, (C₄-C₈)cycloalkylalkyl, fluoro(C₁-C₈)alkyl, fluoro(C₄-C₈)cycloalkylalkyl, (C₁-C₈)alkoxy, (C₄-C₈)cycloalkylalkoxy, fluoro(C₁-C₈)alkoxy, hydroxy(C₁-C₈)alkyl, (C₁-C₅)alkoxy(C₁-C₅)alkyl, halo(C₁-C₅)alkylamino(C₁-C₅)alkyl, (C₁-C₅)alkoxy(C₁-C₅)hydroxyalkyl, (C₃-C₄)cycloalkoxy(C₁-C₅)alkyl, fluoro(C₁-C₅)alkoxy(C₁-C₅)alkyl, fluoro(C₃-C₄)cycloalkoxy(C₁-C₅)alkyl, (C₁-C₅)alkylthio(C₁-C₅)alkyl, (C₁-C₅)alkoxy(C₁-C₅)alkoxy, hydroxy(C₁-C₈)alkoxy, (C₃-C₄)cycloalkoxy(C₁-C₅)alkoxy, fluoro(C₁-C₅)alkoxy(C₁-C₅)alkoxy, fluoro(C₃-C₄)cycloalkoxy(C₁-C₅)alkoxy, (C₁-C₃)alkoxy(C₁-C₃)alkoxy(C₁-C₃)alkyl, fluoro(C₃-C₃)alkoxy(C₁-C₃)alkoxy(C₁-C₃)alkyl, aminocarbonylamino(C₁-C₈)alkyl, aminocarbonylamino(C₁-C₈)alkoxy, (C₁-C₅)alkanoylamino(C₁-C₅)alkyl, (C₁-C₅)alkanoylamino(C₁-C₅)alkoxy, fluoro(C₁-C₅)alkanoylamino(C₁-C₅)alkyl, fluoro(C₁-C₅)alkanoylamino(C₁-C₅)alkoxy, (C₁-C₃)alkoxy(C₁-C₅)alkanoylamino(C₁-C₅)alkyl, (C₁-C₃)alkoxy(C₁-C₅)alkanoylamino(C₁-C₅)alkoxy, (C₃-C₄)cycloalkylcarbonylamino(C₁-C₅)alkyl, (C₃-C₄)cycloalkylcarbonylamino(C₁-C₅)alkoxy, aminosulfonylamino(C₁-C₈)alkyl, aminosulfonylamino(C₁-C₈)alkoxy, (C₁-C₅)alkanesulfonylamino(C₁-C₅)alkyl, (C₁-C₅)alkanesulfonylamino(C₁-C₅)alkoxy, formylamino(C₁-C₅)alkyl, formylamino(C₁-C₅)alkoxy, (C₁-C₅)alkoxycarbonylamino(C₁-C₅)alkyl, (C₁-C₅)alkoxycarbonylamino(C₁-C₅)alkoxy, (C₁-C₅)alkylaminocarbonylamino(C₁-C₅)alkyl, di(C₁-C₅)alkylaminocarbonylamino(C₁-C₅)alkoxy, aminocarbonyl(C₁-C₅)alkyl, aminocarbonyl(C₁-C₅)alkoxy, (C₁-C₅)alkylaminocarbonyl(C₁-C₅)alkyl, (C₁-C₅)alkylaminocarbonyl(C₁-C₅)alkoxy, aminocarboxy(C₁-C₅)alkyl, aminocarboxy(C₁-C₅)alkoxy, (C₁-C₅)alkylaminocarboxy(C₁-C₅)alkyl, (C₁-C₅)alkylaminocarboxy(C₁-C₅)alkoxy, (C₁-C₈)alkoxycarbonylamino, (C₁-C₈)alkylaminocarbonylamino, (C₁-C₈)alkanoylamino, fluoro(C₁-C₈)alkoxycarbonylamino, fluoro(C₁-C₈)alkylaminocarbonylamino, fluoro(C₁-C₈)alkanoylamino, or (C₁-C₈)oxoalkyl.

R³ is H, halogen, —OH, (C₁-C₄)alkanoylamino, or (C₁-C₃)alkoxy, provided that when R³ is —OH or halogen, then R² is not (C₁-C₈)alkoxy, (C₄-C₈)cycloalkylalkoxy, fluoro(C₁-C₈)alkoxy, (C₁-C₅)alkoxy(C₁-C₅)alkoxy, hydroxy(C₁-C₈)alkoxy, (C₃-C₄)cycloalkoxy(C₁-C₅)alkoxy, fluoro(C₁-C₅)alkoxy(C₁-C₅)alkoxy, fluoro(C₃-C₄)cycloalkoxy(C₁-C₅)alkoxy, aminocarbonylamino(C₁-C₈)alkoxy, (C₁-C₅)alkanoylamino(C₁-C₅)alkoxy, fluoro(C₁-C₅)alkanoylamino(C₁-C₅)alkoxy, (C₁-C₃)alkoxy(C₁-C₅)alkanoylamino(C₁-C₅)alkoxy, (C₃-C₄)cycloalkylcarbonylamino(C₁-C₅)alkoxy, aminosulfonylamino(C₁-C₈)alkoxy, (C₁-C₅)alkanesulfonylamino(C₁-C₅)alkoxy, formylamino(C₁-C₅)alkoxy, (C₁-C₅)alkoxycarbonylamino(C₁-C₅)alkoxy, di(C₁-C₅)alkylaminocarbonylamino(C₁-C₅)alkoxy, aminocarbonyl(C₁-C₅)alkoxy, (C₁-C₅)alkylaminocarbonyl(C₁-C₅)alkoxy, aminocarboxy(C₁-C₅)alkoxy, (C₁-C₅)alkylaminocarboxy(C₁-C₅)alkoxy, (C₁-C₈)alkoxycarbonylamino, (C₁-C₈)alkylaminocarbonylamino, (C₁-C₈)alkanoylamino, fluoro(C₁-C₈)alkoxycarbonylamino, fluoro(C₁-C₈)alkylaminocarbonylamino, or fluoro(C₁-C₈)alkanoylamino.

A⁴ is CH₂, O, CH₂CH₂ or CH₂O.

R⁵ and R⁶ are each independently selected from: 1) hydrogen; 2) (C₁-C₁₀)alkyl, (C₃-C₇)cycloalkyl(C₁-C₂)alkyl, (C₄-C₁₀)bicycloalkyl(C₁-C₂)alkyl, (C₈-C₁₂)tricycloalkyl(C₁-C₂)alkyl, saturated heterocyclyl(C₁-C₂)alkyl, (C₁-C₆)alkoxy(C₁-C₆)alkyl, (C₃-C₇)cycloalkoxy(C₁-C₃)alkyl, (C₁-C₅)alkylthio(C₁-C₅)alkyl, or saturated heterocyclyl(C₁-C₃)alkyl, wherein each of these groups are optionally substituted by 1 to 3 groups independently selected from: halogen, cyano, hydroxy, (C₁-C₂)alkyl, (C₁-C₂)alkoxy, (C₃-C₆)cycloalkyl, (C₃-C₆)cycloalkoxy, halo(C₁-C₂)alkyl, halo(C₁-C₂)alkoxy, halo(C₃-C₆)cycloalkyl, and halo(C₃-C₆)cycloalkoxy; and 3) phenyl(C₁-C₂)alkyl or heteroaryl(C₁-C₂)alkyl, wherein each group is optionally substituted with 1 to 3 groups independently selected from: fluoride, chloride, cyano, (C₁-C₃)alkyl, halo(C₁-C₃)alkyl, (C₁-C₃)alkoxy, and halo(C₁-C₃)alkoxy.

Values and particular values for R⁷, R⁹, and the L-G ring are as described in the fifth embodiment.

A seventh embodiment of the present invention is a compound according to Formulae VIa, VIIa, or VIIIa, wherein:

R¹, in Formulae VIa and VIIa, is a saturated heterocycle, phenyl, or heteroaryl; wherein the saturated heterocycle is optionally substituted with 1 to 3 groups independently selected from: fluoride, (C₁-C₃) alkyl, halo(C₁-C₃)alkyl, and oxo, and wherein the phenyl and heteroaryl are optionally and independently substituted with n groups represented by R¹¹, wherein n is an integer from 0-3, and wherein each R¹¹ is independently selected from: halogen, nitro, cyano, (C₁-C₆)alkyl, halo(C₁-C₆)alkyl, (C₁-C₆)alkoxy, halo(C₁-C₆)alkoxy, phenyl and heteroaryl.

R² is (C₁-C₅)alkoxy(C₁-C₅)alkyl, (C₁-C₅)alkoxy(C₁-C₅)hydroxyalkyl, (C₃-C₄)cycloalkoxy(C₁-C₅)alkyl, fluoro(C₁-C₅)alkoxy(C₁-C₅)alkyl, (C₁-C₅)alkoxy(C₁-C₅)alkoxy, fluoro(C₁-C₅)alkoxy(C₁-C₅)alkoxy, (C₁-C₃)alkoxy(C₁-C₃)alkoxy(C₁-C₃)alkyl, fluoro(C₁-C₃)alkoxy(C₁-C₃)alkoxy(C₁-C₃)alkyl, (C₁-C₅)alkanoylamino(C₁-C₅)alkyl, (C₁-C₅)alkanoylamino(C₁-C₅)alkoxy, fluoro(C₁-C₅)alkanoylamino(C₁-C₅)alkyl, fluoro(C₁-C₅)alkanoylamino(C₁-C₅)alkoxy, (C₁-C₃)alkoxy(C₁-C₅)alkanoylamino(C₁-C₅)alkyl, (C₁-C₃)alkoxy(C₁-C₅)alkanoylamino(C₁-C₅)alkoxy, (C₁-C₅)alkoxycarbonylamino(C₁-C₅)alkyl, (C₁-C₅)alkoxycarbonylamino(C₁-C₅)alkoxy, (C₁-C₅)alkylaminocarbonylamino(C₁-C₅)alkyl, di(C₁-C₅)alkylaminocarbonylamino(C₁-C₅)alkoxy, (C₁-C₈)alkylaminocarbonylamino, fluoro(C₁-C₈)alkoxycarbonylamino, or (C₁-C₈)oxoalkyl.

R³ is hydrogen, fluoride, chloride, —OH or (C1-C3)alkoxy, provided that when R³ is —OH, —F, or —Cl, then R² is not (C₁-C₅)alkoxy(C₁-C₅)alkoxy, fluoro(C₁-C₅)alkoxy(C₁-C₅)alkoxy, (C₁-C₅)alkanoylamino(C₁-C₅)alkoxy, fluoro(C₁-C₅)alkanoylamino(C₁-C₅)alkoxy, (C₁-C₃)alkoxy(C₁-C₅)alkanoylamino(C₁-C₅)alkoxy, (C₁-C₅)alkoxycarbonylamino(C₁-C₅)alkoxy, di(C₁-C₅)alkylaminocarbonylamino(C₁-C₅)alkoxy, (C₁-C₈)alkylaminocarbonylamino, or fluoro(C₁-C₈)alkoxycarbonylamino.

A⁴ is CH₂ or O.

R⁵ and R⁶ are each individually hydrogen, (C₃-C₇)cycloalkyl(C₁-C₂)alkyl, saturated heterocyclyl(C₁-C₃)alkyl, (C₃-C₇)cycloalkoxy(C₁-C₃)alkyl, or heteroaryl(C1-C2)alkyl, wherein each group is optionally and individually substituted with 1 to 3 groups selected from: fluoride, chloride, cyano, (C₁-C₃)alkyl, halo(C₁-C₃)alkyl, (C₁-C₃)alkoxy, or halo(C₁-C₃)alkoxy.

The substituents on “L-G ring” are selected from: fluoride, (C₁-C₈)alkyl, halo(C₁-C₈)alkyl, (C₃-C₈)cycloalkyl, halo(C₃-C₈)cycloalkyl, hydroxy(C₃-C₈)cycloalkyl, (C₃-C₈)cycloalkyl(C₁-C₃)alkyl, halo(C₃-C₈)cycloalkyl(C₁-C₃)alkyl, hydroxy(C₃-C₈)cycloalkyl(C₁-C₃)alkyl, (C₁-C₈)alkoxy, halo(C₁-C₈)alkoxy, (C₃-C₈)cycloalkoxy, halo(C₃-C₈)cycloalkoxy, hydroxy(C₃-C₈)cycloalkoxy, (C₁-C₈)alkoxy(C₁-C₃)alkyl, halo(C₁-C₈)alkoxy(C₁-C₃)alkyl, (C₃-C₈)cycloalkoxy(C₁-C₃)alkyl, halo(C₃-c₈)cycloalkoxy(C₁-C₃)alkyl, hydroxy(C₃-C₈)cycloalkoxy(C₁-C₃)alkyl, (C₃-C₈)cycloalkyl(C₁-C₃)alkoxy(C₁-C₃)alkyl, halo(C₃-C₈)cycloalkyl(C₁-C₃)alkoxy(C₁-C₃)alkyl, and hydroxy(C₃-C₈)cycloalkyl(C₁-C₃)alkoxy(C₁-C₃)alkyl.

Values and particular values for R⁷ and R⁹ are as described in the fifth embodiment.

An eighth embodiment of the present invention is a compound according to Formulae VIa, VIIa or VIIIa, wherein R¹ is phenyl optionally substituted with 1 to 3 substituents independently selected from fluoride, chloride and methyl; R² is 4-methoxybutyl, 3-(methoxycarbonylamino)propyl or 2-(methoxycarbonylamino)ethoxy; R³ is —H or —OH; provided that when R² is 2-(methoxycarbonylamino)ethoxy, then R³ is not —OH; A⁴ is CH₂ or O; R⁵ is H or cyclohexylmethyl; R⁶ is H, cyclohexylmethyl; allowing both R⁵ and R⁶ to be H simultaneously; and the “L-G ring” is optionally substituted with one group selected from: (C₃-C₆)alkyl, (C₃-C₆)cycloalkyl, and (C₃-C₆)cycloalkyl(C₁-C₂)alkyl.

More particularly, in the fifth, sixth, seventh, and eighth embodiments, is a compound wherein at least one of R⁵ or R⁶ is hydrogen, and the remainder of the variables are as described above.

Even more particularly, in the fifth, sixth, seventh and eighth embodiments, is a compound wherein at least one of R⁵ or R⁶ is hydrogen, and R² is 4-methoxybutyl, 3-(methoxycarbonylamino)propyl, or 2-(methoxycarbonylamino)ethoxy, and the remainder of the variables are as described above.

The following are compounds of the invention:
(R)—((S)-1-cyclohexyl-3-(methylamino)propan-2-yl)-3-((S)-1-(3-chloro-2-fluorophenyl)-1-hydroxy-5-methoxypentyl)piperidine-1-carboxylate;

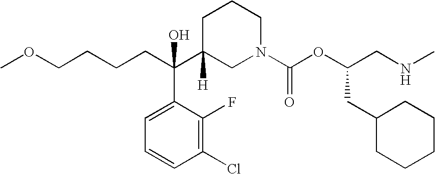

(R)—((S)-1-cyclohexyl-3-(methylamino)propan-2-yl)-3-((S)-1-(3-chlorophenyl)-1-hydroxy-4-(methoxycarbonylamino)butyl)piperidine-1-carboxylate;

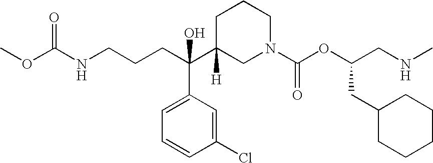

(R)—((S)-1-cyclohexyl-3-(methylamino)propan-2-yl)-2-((R)-1-(3-chloro-2-fluorophenyl)-1-hydroxy-5-methoxypentyl)morpholine-4-carboxylate;

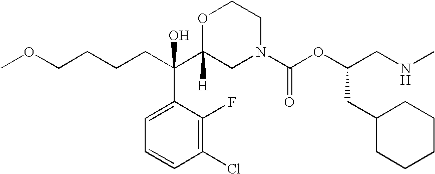

(R)—((S)-2-amino-3-cyclohexylpropyl)-3-((S)-1-(3-chloro-2-fluorophenyl)-1-hydroxy-5-methoxypentyl)piperidine-1-carboxylate;

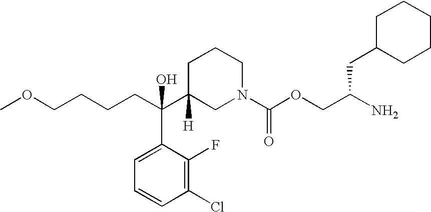

(R)—((S)-2-amino-3-((R)-tetrahydro-2H-pyran-3-yl)propyl)-3-((R)-(3-chlorophenyl)(2-(methoxycarbonylamino)ethoxy)methyl)piperidine-1-carboxylate;

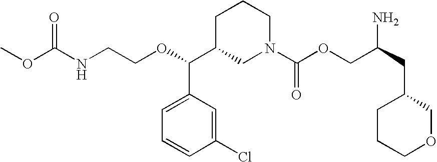

(R)—((R)-1-cyclohexyl-3-(methylamino)propan-2-yl)-3-((S)-1-(3-chlorophenyl)-1-hydroxy-4-(methoxycarbonylamino)butyl)piperidine-1-carboxylate;

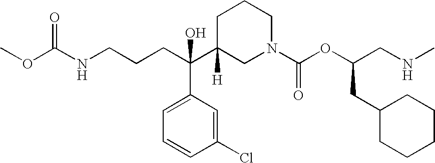

(R)-((3SR,4RS)-4-isobutylpiperidin-3-yl)-3-(R)-(3-chlorophenyl)(2-(methoxycarbonylamino)ethoxy)methyl)piperidine-1-carboxylate;

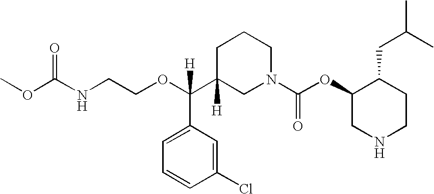

(R)—((S)-3-(cyclohexylmethyl)pyrrolidin-3-yl)-3-((R)-(3-chlorophenyl)(2-(methoxycarbonylamino)ethoxy)methyl)piperidine-1-carboxylate;

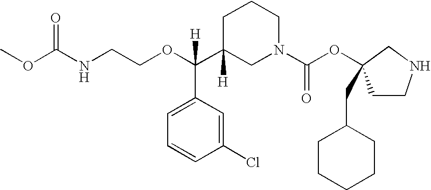

(R)—((R)-3-(cyclohexylmethyl)pyrrolidin-3-yl)-3-((R)-(3-chlorophenyl)(2-(methoxycarbonylamino)ethoxy)methyl)piperidine-1-carboxylate;

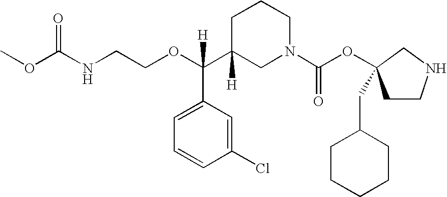

(R)—((R)-1-cyclohexyl-3-(methylamino)propan-2-yl)-3-((S)-1-(3-chloro-2-fluorophenyl)-1-hydroxy-5-methoxypentyl)piperidine-1-carboxylate; and

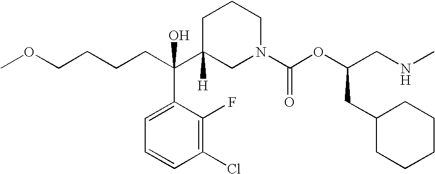

(R)—((R)-1-cyclohexyl-3-(methylamino)propan-2-yl)-2-((R)-1-(3-chloro-2-fluorophenyl)-1-hydroxy-5-methoxypentyl)morpholine-4-carboxylate.

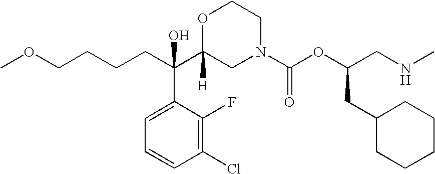

Preferred compounds of the present invention are:
(R)—((S)-1-cyclohexyl-3-(methylamino)propan-2-yl)-3-((S)-1-(3-chloro-2-fluorophenyl)-1-hydroxy-5-methoxypentyl)piperidine-1-carboxylate;
(R)-((S)-1-cyclohexyl-3-(methylamino)propan-2-yl)-3-((S)-1-(3-chlorophenyl)-1-hydroxy-4-(methoxycarbonylamino)butyl)piperidine-1-carboxylate;
(R)—((S)-1-cyclohexyl-3-(methylamino)propan-2-yl)-2-((R)-1-(3-chloro-2-fluorophenyl)-1-hydroxy-5-methoxypentyl)morpholine-4-carboxylate;
(R)—((S)-2-amino-3-cyclohexylpropyl)-3-((S)-1-(3-chloro-2-fluorophenyl)-1-hydroxy-5-methoxypentyl)piperidine-1-carboxylate; and
(R)—((S)-2-amino-3-((R)-tetrahydro-2H-pyran-3-yl)propyl)-3-((R)-(3-chlorophenyl)(2-(methoxycarbonylamino)ethoxy)methyl)piperidine-1-carboxylate.

More preferred compounds of the present invention are:
(R)—((S)-1-cyclohexyl-3-(methylamino)propan-2-yl)-3-((S)-1-(3-chlorophenyl)-1-hydroxy-4-(methoxycarbonylamino)butyl)piperidine-1-carboxylate; and
(R)—((S)-2-amino-3-((R)-tetrahydro-2H-pyran-3-yl)propyl)-3-((R)-(3-chlorophenyl)(2-(methoxycarbonylamino)ethoxy)methyl)piperidine-1-carboxylate.

When any variable (e.g., aryl, heterocyclyl, R¹, R², etc.) occurs more than once in a compound, its definition on each occurrence is independent of any other occurrence.

“Alkyl” means a saturated aliphatic branched or straight-chain mono- or di-valent hydrocarbon radical having the specified number of carbon atoms. Thus, “(C₁-C₈)alkyl” means a radical having from 1-8 carbon atoms in a linear or branched arrangement. “(C₁-C₆)alkyl” includes methyl, ethyl, propyl, butyl, pentyl, and hexyl.

“Alkylene” means a saturated aliphatic straight-chain divalent hydrocarbon radical having the specified number of carbon atoms, e.g., —(CH₂)ₓ— wherein x is a positive integer such as 1-10, preferably 1-6. Thus, “(C₁-C₆)alkylene” means a radical having from 1-6 carbon atoms in a linear or branched arrangement, with optional unsaturation or optional substitution.

“Cycloalkyl” means a saturated aliphatic cyclic hydrocarbon radical having the specified number of carbon atoms. Thus, (C₃-C₇)cycloalkyl means a radical having from 3-8 carbon atoms arranged in a ring. (C₃-C₇)cycloalkyl includes cyclopropyl, cyclobutyl, cyclopentyl, cyclohexyl, and cycloheptyl.

Haloalkyl and halocycloalkyl include mono, poly, and perhaloalkyl groups where the halogens are independently selected from fluorine, chlorine, and bromine.

Saturated heterocyclic rings are 4-, 5-, 6-, and 7-membered heterocyclic rings containing 1 to 4 heteroatoms independently selected from N, O, and S, and include pyrrolidine, piperidine, tetrahydrofuran, tetrahydropyran, tetrahydrothiophene, tetrahydrothiopyran, isoxazolidine, 1,3-dioxolane, 1,3-dithiolane, 1,3-dioxane, 1,4-dioxane, 1,3-dithiane, 1,4-dithiane, morpholine, thiomorpholine, thiomorpholine 1,1-dioxide, tetrahydro-2H-1,2-thiazine 1,1-dioxide, and isothiazolidine 1,1-dioxide. Oxo substituted saturated heterocyclic rings include tetrahydrothiophene 1-oxide, tetrahydrothiophene 1,1-dioxide, thiomorpholine 1-oxide, thiomorpholine 1,1-dioxide, tetrahydro-2H-1,2-thiazine 1,1-dioxide, and isothiazolidine 1,1-dioxide, pyrrolidin-2-one, piperidin-2-one, piperazin-2-one, and morpholin-2-one.

“Heteroaryl” means a monovalent heteroaromatic monocyclic or polycylic ring radical. Heteroaryl rings are 5- and 6-membered aromatic heterocyclic rings containing 1 to 4 heteroatoms independently selected from N, O, and S, and include furan, thiophene, pyrrole, imidazole, pyrazole, oxazole, isoxazole, thiazole, isothiazole, 1,2,3-triazole, 1,2,4-triazole, 1,3,4-oxadiazole, 1,2,5-thiadiazole, 1,2,5-thiadiazole 1-oxide, 1,2,5-thiadiazole 1,1-dioxide, 1,3,4-thiadiazole, pyridine, pyridine-N-oxide, pyrazine, pyrimidine, pyridazine, 1,2,4-triazine, 1,3,5-triazine, and tetrazole. Bicyclic heteroaryl rings are bicyclo[4.4.0] and bicyclo[4,3.0] fused ring systems containing 1 to 4 heteroatoms independently selected from N, O, and S, and include indolizine, indole, isoindole, benzo[b]furan, benzo[b]thiophene, indazole, benzimidazole, benzthiazole, purine, 4H-quinolizine, quinoline, isoquinoline, cinnoline, phthalazine, quinazoline, quinoxaline, 1,8-naphthyridine, and pteridine.

Bicycloalkyl rings can be fused, bridged or spiro ring systems, and include bicyclo[1.1.0]butane, bicyclo[1.2.0]pentane, bicyclo[2.2.0]hexane, bicyclo[3.2.0]heptane, bicyclo[3.3.0]octane, bicyclo[4.2.0]octane, bicyclo[2.2.1]heptane, bicyclo[2.2.2]octane, bicyclo[3.2.1]octane, bicyclo[3.2.2]nonane, bicyclo[3.3.1]nonane, bicyclo[3.3.2]decane and bicyclo[3.3.3]undecane, spiro[2.2]pentane, spiro[2.3]hexane, spiro[3.3]heptane, spiro[2.4]heptane, spiro[3.4]octane, and spiro[2.5]octane.

Tricycloalkyl rings can be fused, bridged or spiro ring systems, and include tricyclo[3.3.1.0^{3,7}]nonane (noradamantane) and tricyclo[3.3.1.1^{3,7}]decane (adamantane).

“Alkoxy” means an alkyl radical attached through an oxygen linking atom. “(C₁-C₄)-alkoxy” includes methoxy, ethoxy, propoxy, and butoxy.

“Aromatic” means an unsaturated cycloalkyl ring system.

“Aryl” means an aromatic monocyclic, or polycyclic ring system. Aryl systems include phenyl, naphthalenyl, fluorenyl, indenyl, azulenyl, and anthracenyl.

“Hetero” refers to the replacement of at least one carbon atom member in a ring system with at least one heteroatom selected from N, S, and O. A hetero ring may have 1, 2, 3, or 4 carbon atom members replaced by a heteroatom.

“Unsaturated ring” means a ring containing one or more double bonds and include cyclopentene, cyclohexene, cyclopheptene, cyclohexadiene, benzene, pyrroline, pyrazole, 4,5-dihydro-1H-imidazole, imidazole, 1,2,3,4-tetrahydropyridine, 1,2,3,6-tetrahydropyridine, pyridine and pyrimidine.

### Enantiomers, Diastereomers, and Salts

Certain compounds of Formula I may exist in various stereoisomeric or tautomeric forms. The invention encompasses all such forms, including active compounds in the form of essentially pure enantiomers, racemic mixtures, and tautomers, including forms those not depicted structurally.

The compounds of the invention may be present in the form of pharmaceutically acceptable salts. For use in medicines, the salts of the compounds of the invention refer to non-toxic “pharmaceutically acceptable salts.” Pharmaceutically acceptable salt forms include pharmaceutically acceptable acidic/anionic or basic/cationic salts.

Pharmaceutically acceptable acidic/anionic salts include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphospate, polygalacturonate, salicylate, stearate, subacetate, succinate, sulfate, tannate, tartrate, teoclate, tosylate, and triethiodide salts.

The compounds of the invention include pharmaceutically acceptable anionic salt forms, wherein the anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphospate, polygalacturonate, salicylate, stearate, subacetate, succinate, sulfate, tannate, tartrate, teoclate, tosylate, and triethiodide salts.

The anionic salt form of a compound of the invention includes the acetate, bromide, camsylate, chloride, edisylate, fumarate, hydrobromide, hydrochloride, iodide, isethionate, lactate, mesylate, maleate, napsylate, salicylate, sulfate, and tosylate salts.

When a disclosed compound or its pharmaceutically acceptable salt is named or depicted by structure, it is to be understood that solvates or hydrates of the compound or its pharmaceutically acceptable salts are also included. “Solvates” refer to crystalline forms wherein solvent molecules are incorporated into the crystal lattice during crystallization. Solvate may include water or nonaqueous solvents such as ethanol, isopropanol, DMSO, acetic acid, ethanolamine, and EtOAc. Solvates, wherein water is the solvent molecule incorporated into the crystal lattice, are typically referred to as “hydrates”. Hydrates include stoichiometric hydrates as well as compositions containing variable amounts of water.

When a disclosed compound or its pharmaceutically acceptable salt is named or depicted by structure, it is to be understood that the compound, including solvates thereof, may exist in crystalline forms, non-crystalline forms or a mixture thereof. The compound or its pharmaceutically acceptable salts or solvates may also exhibit polymorphism (i.e. the capacity to occur in different crystalline forms). These different crystalline forms are typically known as “polymorphs.” It is to be understood that when named or depicted by structure, the disclosed compound and its pharmaceutically acceptable salts, solvates or hydrates also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in solidifying the compound. For example, changes in temperature, pressure, or solvent may result in different polymorphs. In addition, one polymorph may spontaneously convert to another polymorph under certain conditions.

It may be necessary and/or desirable during synthesis to protect sensitive or reactive groups on any of the molecules concerned. Representative conventional protecting groups are described in T. W. Greene and P. G. M. Wuts “Protective Groups in Organic Synthesis” John Wiley & Sons, Inc., New York 1999. Protecting groups may be added and removed using methods well known in the art.

The invention also includes various isomers and mixtures thereof. “Isomer” refers to compounds that have the same composition and molecular weight but differ in physical and/or chemical properties. The structural difference may be in constitution (geometric isomers) or in the ability to rotate the plane of polarized light (stereoisomers).

Certain of the disclosed aspartic protease inhibitors may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms. The symbol “*” in a structural formula represents the presence of a chiral carbon center. “R” and “S” represent the configuration of substituents around one or more chiral carbon atoms. Thus, “R*” and “S*” denote the relative configurations of substituents around one or more chiral carbon atoms. When a chiral center is not defined as R or S, a mixture of both configurations is present.

“Racemate” or “racemic mixture” means a compound of equimolar quantities of two enantiomers, wherein such mixtures exhibit no optical activity; i.e., they do not rotate the plane of polarized light.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration.

Atoms (other than H) attached to a carbocyclic ring may be in a cis or trans configuration. In the “cis” configuration, the substituents are on the same side in relationship to the plane of the ring; in the “trans” configuration, the substituents are on opposite sides in relationship to the plane of the ring. A mixture of “cis” and “trans” species is designated “cis/trans”.

The point at which a group or moiety is attached to the remainder of the compound or another group or moiety can be indicated by [symbol] which represents [symbol] or

[symbol]

“R,” “S,” “S*,” “R*,” “E,” “Z,” “cis,” and “trans,” indicate configurations relative to the core molecule.

The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least 60%, 70%, 80%, 90%, 99% or 99.9% by weight pure relative to the other stereoisomers. When a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

When a disclosed compound is named or depicted by structure without indicating the stereochemistry, and the inhibitor has at least one chiral center, it is to be understood that the name or structure encompasses one enantiomer of inhibitor free from the corresponding optical isomer, a racemic mixture of the inhibitor and mixtures enriched in one enantiomer relative to its corresponding optical isomer.

When a disclosed aspartic protease inhibitor is named or depicted by structure without indicating the stereochemistry and has at least two chiral centers, it is to be understood that the name or structure encompasses a diastereomer free of other diastereomers, a pair of diastereomers free from other diastereomeric pairs, mixtures of diastereomers, mixtures of diastereomeric pairs, mixtures of diastereomers in which one diastereomer is enriched relative to the other diastereomer(s) and mixtures of diastereomeric pairs in which one diastereomeric pair is enriched relative to the other diastereomeric pair(s).

The compounds of the invention are useful for ameliorating or treating disorders or diseases in which decreasing the levels of aspartic protease products is effective in treating the disease state or in treating infections in which the infectious agent depends upon the activity of an aspartic protease. In hypertension, elevated levels of angiotensin I, the product of renin catalyzed cleavage of angiotensinogen are present. Thus, the compounds of the invention can be used in the treatment of hypertension, heart failure such as (acute and chronic) congestive heart failure; left ventricular dysfunction; cardiac hypertrophy; cardiac fibrosis; cardiomyopathy (e.g., diabetic cardiac myopathy and post-infarction cardiac myopathy); supraventricular and ventricular arrhythmias; atrial fibrillation; atrial flutter; detrimental vascular remodeling; myocardial infarction and its sequelae; atherosclerosis; angina (whether unstable or stable); renal failure conditions, such as diabetic nephropathy; glomerulonephritis; renal fibrosis; scleroderma; glomerular sclerosis; microvascular complications, for example, diabetic retinopathy; renal vascular hypertension; vasculopathy; neuropathy; complications resulting from diabetes, including nephropathy, vasculopathy, retinopathy and neuropathy, diseases of the coronary vessels, proteinuria, albumenuria, post-surgical hypertension, metabolic syndrome, obesity, restenosis following angioplasty, eye diseases and associated abnormalities including raised intra-ocular pressure, glaucoma, retinopathy, abnormal vascular growth and remodelling, angiogenesis-related disorders, such as neovascular age related macular degeneration; hyperaldosteronism, anxiety states, and cognitive disorders (Fisher N. D.; Hollenberg N. K. Expert Opin. Investig. Drugs. 2001, 10, 417-26).

Elevated levels of β-amyloid, the product of the activity of the well-characterized aspartic protease β-secretase (BACE) activity on amyloid precursor protein, are widely believed to be responsible for the development and progression of amyloid plaques in the brains of Alzheimer's disease patients. The secreted aspartic proteases of Candida albicans are associated with its pathogenic virulence (Naglik, J. R.; Challacombe, S. J.; Hube, B. Microbiology and Molecular Biology Reviews 2003, 67, 400-428). The viruses HIV and HTLV depend on their respective aspartic proteases for viral maturation. Plasmodium falciparum uses plasmepsins I and II to degrade hemoglobin.

A pharmaceutical composition of the invention may, alternatively or in addition to a compound of Formula I, comprise a pharmaceutically acceptable salt of a compound of Formula I or a prodrug or pharmaceutically active metabolite of such a compound or salt and one or more pharmaceutically acceptable carriers therefor.

The compositions of the invention are aspartic protease inhibitors. Said compositions contain compounds having a mean inhibition constant (IC₅₀) against aspartic proteases of between about 5,000 nM to about 0.01 nM; preferably between about 50 nM to about 0.01 nM; and more preferably between about 5 nM to about 0.01 nM.

The compositions of the invention reduce blood pressure. Said compositions include compounds having an IC₅₀ for renin of between about 5,000 nM to about 0.01 nM; preferably between about 50 nM to about 0.01 nM; and more preferably between about 5 nM to about 0.01 nM.

The invention includes a therapeutic method for treating or ameliorating an aspartic protease mediated disorder in a subject in need thereof comprising administering to a subject in need thereof an effective amount of a compound of Formula I, or the enantiomers, diastereomers, or salts thereof or composition thereof.

Administration methods include administering an effective amount (i.e., a therapeutically effective amount) of a compound or composition of the invention at different times during the course of therapy or concurrently in a combination form. The methods of the invention include all known therapeutic treatment regimens.

“Prodrug” means a pharmaceutically acceptable form of an effective derivative of a compound (or a salt thereof) of the invention, wherein the prodrug may be: 1) a relatively active precursor which converts in vivo to a compound of the invention; 2) a relatively inactive precursor which converts in vivo to a compound of the invention; or 3) a relatively less active component of the compound that contributes to therapeutic activity after becoming available in vivo (i.e., as a metabolite). See “Design of Prodrugs”, ed. H. Bundgaard, Elsevier, 1985.

“Metabolite” means a pharmaceutically acceptable form of a metabolic derivative of a compound (or a salt thereof) of the invention, wherein the derivative is an active compound that contributes to therapeutic activity after becoming available in vivo.

“Effective amount” means that amount of active compound agent that elicits the desired biological response in a subject. Such response includes alleviation of the symptoms of the disease or disorder being treated. The effective amount of a compound of the invention in such a therapeutic method is from about 10 mg/kg/day to about 0.01 mg/kg/day, preferably from about 0.5 mg/kg/day to 5 mg/kg/day.

The invention includes the use of a compound of the invention for the preparation of a composition for treating or ameliorating an aspartic protease mediated chronic disorder or disease or infection in a subject in need thereof, wherein the composition comprises a mixture one or more compounds of the invention and an optional pharmaceutically acceptable carrier.

“Pharmaceutically acceptable carrier” means compounds and compositions that are of sufficient purity and quality for use in the formulation of a composition of the invention and that, when appropriately administered to an animal or human, do not produce an adverse reaction.

“Aspartic protease mediated disorder or disease” includes disorders or diseases associated with the elevated expression or overexpression of aspartic proteases and conditions that accompany such diseases.

An embodiment of the invention includes administering a renin inhibiting compound of Formula I or composition thereof in a combination therapy (U.S. Pat. No. 5,821,232; U.S. Pat. No. 6,716,875; U.S. Pat. No. 5,663,188; Fossa, A. A. et al., Synergistic effect on reduction in blood pressure with coadministration of a renin inhibitor or an angiotensin-converting enzyme inhibitor with an angiotensin II receptor antagonist, Drug Development Research, 1994, 33(4), 422-8) with one or more additional agents for the treatment of hypertension including α-blockers, β-blockers, calcium channel blockers, diuretics, natriuretics, saluretics, centrally acting antiphypertensives, angiotensin converting enzyme (ACE) inhibitors, dual ACE and neutral endopeptidase (NEP) inhibitors, angiotensin-receptor blockers (ARBs), aldosterone synthase inhibitor, aldosterone-receptor antagonists, or endothelin receptor antagonist.

α-Blockers include doxazosin, prazosin, tamsulosin, and terazosin.

β-Blockers for combination therapy are selected from atenolol, bisoprol, metoprolol, acetutolol, esmolol, celiprolol, taliprolol, acebutolol, oxprenolol, pindolol, propanolol, bupranolol, penbutolol, mepindolol, carteolol, nadolol, carvedilol, and their pharmaceutically acceptable salts.

Calcium channel blockers include dihydropyridines (DHPs) and non-DHPs. The preferred DHPs are selected from the group consisting of amlodipine, felodipine, ryosidine, isradipine, lacidipine, nicardipine, nifedipine, nigulpidine, niludipine, nimodiphine, nisoldipine, nitrendipine, and nivaldipine and their pharmaceutically acceptable salts. Non-DHPs are selected from flunarizine, prenylamine, diltiazem, fendiline, gallopamil, mibefradil, anipamil, tiapamil, and verampimil and their pharmaceutically acceptable salts.

A diuretic is, for example, a thiazide derivative selected from amiloride, chlorothiazide, hydrochlorothiazide, methylchlorothiazide, and chlorothalidon.

ACE inhibitors include alacepril, benazepril, benazaprilat, captopril, ceronapril, cilazapril, delapril, enalapril, enalaprilat, fosinopril, lisinopril, moexipiril, moveltopril, perindopril, quinapril, quinaprilat, ramipril, ramiprilat, spirapril, temocapril, trandolapril, and zofenopril. Preferred ACE inhibitors are benazepril, enalpril, lisinopril, and ramipril.

Dual ACE/NEP inhibitors are, for example, omapatrilat, fasidotril, and fasidotrilat.

Preferred ARBs include candesartan, eprosartan, irbesartan, losartan, olmesartan, tasosartan, telmisartan, and valsartan.

Preferred aldosterone synthase inhibitors are anastrozole, fadrozole, and exemestane.

Preferred aldosterone-receptor antagonists are spironolactone and eplerenone.

A preferred endothelin antagonist is, for example, bosentan, enrasentan, atrasentan, darusentan, sitaxentan, and tezosentan and their pharmaceutically acceptable salts.

An embodiment of the invention includes administering an HIV protease inhibiting compound of Formula I or composition thereof in a combination therapy with one or more additional agents for the treatment of AIDS reverse transcriptase inhibitors, non-nucleoside reverse transcriptase inhibitors, other HIV protease inhibitors, HIV integrase inhibitors, entry inhibitors (including attachment, co-receptor and fusion inhibitors), antisense drugs, and immune stimulators.

Preferred reverse transcriptase inhibitors are zidovudine, didanosine, zalcitabine, stavudine, lamivudine, abacavir, tenofovir, and emtricitabine.

Preferred non-nucleoside reverse transcriptase inhibitors are nevirapine, delaviridine, and efavirenz.

Preferred HIV protease inhibitors are saquinavir, ritonavir, indinavir, nelfinavir, amprenavir, lopinavir, atazanavir, and fosamprenavir.

Preferred HIV integrase inhibitors are L-870,810 and S-1360.

Entry inhibitors include compounds that bind to the CD4 receptor, the CCR5 receptor or the CXCR4 receptor. Specific examples of entry inhibitors include enfuvirtide (a peptidomimetic of the HR2 domain in gp41) and sifurvitide.

A preferred attachment and fusion inhibitor is enfuvirtide.

An embodiment of the invention includes administering β-secretase inhibiting compound of Formula I or composition thereof in a combination therapy with one or more additional agents for the treatment of Alzheimer's disease including tacrine, donepezil, rivastigmine, galantamine, and memantine.

An embodiment of the invention includes administering a plasmepsin inhibiting compound of Formula I or composition thereof in a combination therapy with one or more additional agents for the treatment of malaria including artemisinin, chloroquine, halofantrine, hydroxychloroquine, mefloquine, primaquine, pyrimethamine, quinine, sulfadoxine.

Combination therapy includes co-administration of the compound of the invention and said other agent, sequential administration of the compound and the other agent, administration of a composition containing the compound and the other agent, or simultaneous administration of separate compositions containing of the compound and the other agent.

The invention further includes the process for making the composition comprising mixing one or more of the present compounds and an optional pharmaceutically acceptable carrier; and includes those compositions resulting from such a process, which process includes conventional pharmaceutical techniques.

The compositions of the invention include ocular, oral, nasal, transdermal, topical with or without occlusion, intravenous (both bolus and infusion), and injection (intraperitoneally, subcutaneously, intramuscularly, intratumorally, or parenterally). The composition may be in a dosage unit such as a tablet, pill, capsule, powder, granule, liposome, ion exchange resin, sterile ocular solution, or ocular delivery device (such as a contact lens and the like facilitating immediate release, timed release, or sustained release), parenteral solution or suspension, metered aerosol or liquid spray, drop, ampoule, auto-injector device, or suppository; for administration ocularly, orally, intranasally, sublingually, parenterally, or rectally, or by inhalation or insufflation.

Compositions of the invention suitable for oral administration include solid forms such as pills, tablets, caplets, capsules (each including immediate release, timed release, and sustained release formulations), granules and powders; and, liquid forms such as solutions, syrups, elixirs, emulsions, and suspensions. Forms useful for ocular administration include sterile solutions or ocular delivery devices. Forms useful for parenteral administration include sterile solutions, emulsions, and suspensions.

The compositions of the invention may be administered in a form suitable for once-weekly or once-monthly administration. For example, an insoluble salt of the active compound may be adapted to provide a depot preparation for intramuscular injection (e.g., a decanoate salt) or to provide a solution for ophthalmic administration.

The dosage form containing the composition of the invention contains a therapeutically effective amount of the active ingredient necessary to provide a therapeutic effect. The composition may contain from about 5,000 mg to about 0.5 mg (preferably, from about 1,000 mg to about 0.5 mg) of a compound of the invention or salt form thereof and may be constituted into any form suitable for the selected mode of administration. The composition may be administered about 1 to about 5 times per day. Daily administration or post-periodic dosing may be employed.

For oral administration, the composition is preferably in the form of a tablet or capsule containing, e.g., 500 to 0.5 milligrams of the active compound. Dosages will vary depending on factors associated with the particular patient being treated (e.g., age, weight, diet, and time of administration), the severity of the condition being treated, the compound being employed, the mode of administration, and the strength of the preparation.

The oral composition is preferably formulated as a homogeneous composition, wherein the active ingredient is dispersed evenly throughout the mixture, which may be readily subdivided into dosage units containing equal amounts of a compound of the invention. Preferably, the compositions are prepared by mixing a compound of the invention (or pharmaceutically acceptable salt thereof) with one or more optionally present pharmaceutical carriers (such as a starch, sugar, diluent, granulating agent, lubricant, glidant, binding agent, and disintegrating agent), one or more optionally present inert pharmaceutical excipients (such as water, glycols, oils, alcohols, flavoring agents, preservatives, coloring agents, and syrup), one or more optionally present conventional tableting ingredients (such as corn starch, lactose, sucrose, sorbitol, talc, stearic acid, magnesium stearate, dicalcium phosphate, and any of a variety of gums), and an optional diluent (such as water).

Binder agents include starch, gelatin, natural sugars (e.g., glucose and beta-lactose), corn sweeteners and natural and synthetic gums (e.g., acacia and tragacanth). Disintegrating agents include starch, methyl cellulose, agar, and bentonite.

Tablets and capsules represent an advantageous oral dosage unit form. Tablets may be sugarcoated or film-coated using standard techniques. Tablets may also be coated or otherwise compounded to provide a prolonged, control-release therapeutic effect. The dosage form may comprise an inner dosage and an outer dosage component, wherein the outer component is in the form of an envelope over the inner component. The two components may further be separated by a layer that resists disintegration in the stomach (such as an enteric layer) and permits the inner component to pass intact into the duodenum or a layer which delays or sustains release. A variety of enteric and non-enteric layer or coating materials (such as polymeric acids, shellacs, acetyl alcohol, and cellulose acetate or combinations thereof) may be used.

Compounds of the invention may also be administered via a slow release composition; wherein the composition includes a compound of the invention and a biodegradable slow release carrier (e.g., a polymeric carrier) or a pharmaceutically acceptable non-biodegradable slow release carrier (e.g., an ion exchange carrier).

Biodegradable and non-biodegradable slow release carriers are well known in the art. Biodegradable carriers are used to form particles or matrices which retain an active agent(s) and which slowly degrade/dissolve in a suitable environment (e.g., aqueous, acidic, basic and the like) to release the agent. Such particles degrade/dissolve in body fluids to release the active compound(s) therein. The particles are preferably nanoparticles (e.g., in the range of about 1 to 500 nm in diameter, preferably about 50-200 nm in diameter, and most preferably about 100 nm in diameter). In a process for preparing a slow release composition, a slow release carrier and a compound of the invention are first dissolved or dispersed in an organic solvent. The resulting mixture is added into an aqueous solution containing an optional surface-active agent(s) to produce an emulsion. The organic solvent is then evaporated from the emulsion to provide a colloidal suspension of particles containing the slow release carrier and the compound of the invention.

The compound of Formula I may be incorporated for administration orally or by injection in a liquid form such as aqueous solutions, suitably flavored syrups, aqueous or oil suspensions, flavored emulsions with edible oils such as cottonseed oil, sesame oil, coconut oil or peanut oil and the like, or in elixirs or similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone, and gelatin. The liquid forms in suitably flavored suspending or dispersing agents may also include synthetic and natural gums. For parenteral administration, sterile suspensions and solutions are desired. Isotonic preparations, which generally contain suitable preservatives, are employed when intravenous administration is desired.

The compounds may be administered parenterally via injection. A parenteral formulation may consist of the active ingredient dissolved in or mixed with an appropriate inert liquid carrier. Acceptable liquid carriers usually comprise aqueous solvents and other optional ingredients for aiding solubility or preservation. Such aqueous solvents include sterile water, Ringer's solution, or an isotonic aqueous saline solution. Other optional ingredients include vegetable oils (such as peanut oil, cottonseed oil, and sesame oil), and organic solvents (such as solketal, glycerol, and formyl). A sterile, non-volatile oil may be employed as a solvent or suspending agent. The parenteral formulation is prepared by dissolving or suspending the active ingredient in the liquid carrier whereby the final dosage unit contains from 0.005 to 10% by weight of the active ingredient. Other additives include preservatives, isotonizers, solubilizers, stabilizers, and pain-soothing agents. Injectable suspensions may also be prepared, in which case appropriate liquid carriers, suspending agents and the like may be employed.

Compounds of the invention may be administered intranasally using a suitable intranasal vehicle.

Compounds of the invention may also be administered topically using a suitable topical transdermal vehicle or a transdermal patch.

For ocular administration, the composition is preferably in the form of an ophthalmic composition. The ophthalmic compositions are preferably formulated as eye-drop formulations and filled in appropriate containers to facilitate administration to the eye, for example a dropper fitted with a suitable pipette. Preferably, the compositions are sterile and aqueous based, using purified water. In addition to the compound of the invention, an ophthalmic composition may contain one or more of: a) a surfactant such as a polyoxyethylene fatty acid ester; b) a thickening agents such as cellulose, cellulose derivatives, carboxyvinyl polymers, polyvinyl polymers, and polyvinylpyrrolidones, typically at a concentration n the range of about 0.05 to about 5.0% (wt/vol); c) (as an alternative to or in addition to storing the composition in a container containing nitrogen and optionally including a free oxygen absorber such as Fe), an anti-oxidant such as butylated hydroxyanisol, ascorbic acid, sodium thiosulfate, or butylated hydroxytoluene at a concentration of about 0.00005 to about 0.1% (wt/vol); d) ethanol at a concentration of about 0.01 to 0.5% (wt/vol); and e) other excipients such as an isotonic agent, buffer, preservative, and/or pH-controlling agent. The pH of the ophthalmic composition is desirably within the range of 4-8.

### Methods of Preparation

In the discussion below, R¹, R², R³, X, Y, A, L, R⁵, G, R⁹ and R¹⁰ are defined as described above for compounds of Formula I. In cases where the synthetic intermediates and final products of Formula I described below contain potentially reactive functional groups, for example amino, hydroxyl, thiol and carboxylic acid groups, that may interfere with the desired reaction, it may be advantageous to employ protected forms of the intermediate. Methods for the selection, introduction and subsequent removal of protecting groups are well known to those skilled in the art. (T. W. GREENE & P. G. M. WUTS, PROTECTIVE GROUPS IN ORGANIC SYNTHESIS, John Wiley & Sons, Inc., New York 1999). Such protecting group manipulations are assumed in the discussion below and not usually described explicitly. Generally, reagents in the reaction schemes are used in equimolar amounts; however, in certain cases it may be desirable to use an excess of one reagent to drive a reaction to completion. This is especially the case when the excess reagent can be readily removed by evaporation or extraction. Bases employed to neutralize HCI in reaction mixtures are generally used in slight to substantial excess (1.05 to 5 equivalents).

In the first process, a compound of Formula I is prepared by reaction of an intermediate of Formula II wherein Z¹ is a leaving group such as halide, alkanesulfonate, haloalkanesulfonate, arylsulfonate, aryloxide, heteroaryloxide, azole or azolium salt with an alcohol intermediate of Formula III:

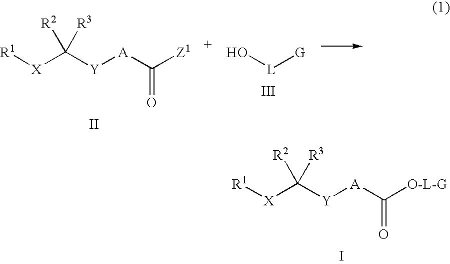

When C(═O)Z¹ is attached to a nitrogen that is part of ring A, the reaction can be effected using a strong base such as NaH in an ethereal solvent such as THF at 25° C. to 150° C., or using a soluble base such as pyridine or collidine as solvent or co-solvent at 50° C. to 200° C. When C(═O)Z¹ is attached to a carbon atom that is part of ring A, the reaction can be effected using a soluble base such as TEA or DIEA in an inert solvent such as CH₂Cl₂ or THF at −20° C. to 50° C.

In the second process, a compound of Formula I, wherein the carbonyl carbon is attached to a nitrogen atom that is part of ring A, is prepared by reaction of a compound of cyclic secondary amine of Formula IV, wherein the H atom in IV is attached to a nitrogen atom that is part of ring A, with a compound of Formula V, wherein Z¹ is a leaving group such as halide, alkanesulfonate, arylsulfonate, aryloxide, azole or azolium salt:

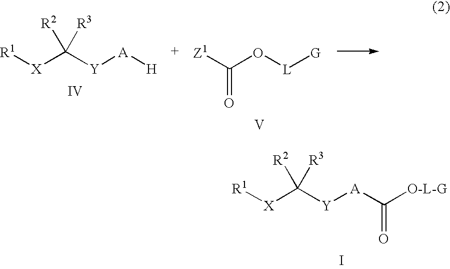

In the third process, a compound of Formula I, wherein G is —NHR⁹ or —NR⁹R¹⁰, is prepared by reductive amination of an aldehyde of Formula VI wherein L* is a linear (C₁-C₃)alkyl chain with an amine of formula R^{e}NH₂ or R^{e}R^{f}NH using a reducing agent such as NaCNBH₃ or NaBH(OAc)₃:

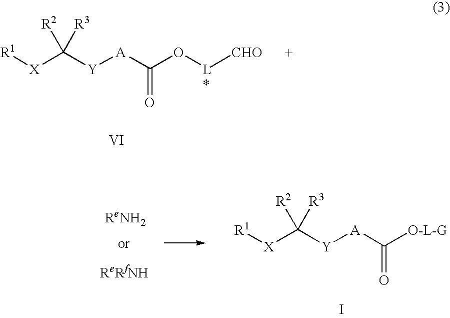

In the fourth process, a compound of Formula I, wherein G is —NH₂, is prepared by reduction of an azide of Formula VI using catalytic hydrogenation or PPh₃ in wet THF:

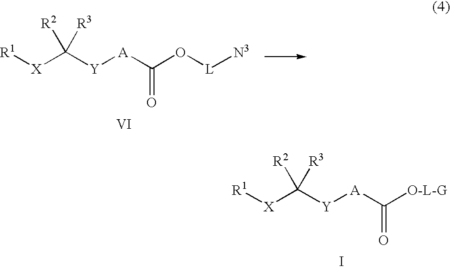

In the fifth process, optionally protected compounds of Formula I, wherein R³ is —OH, are prepared from ketone compounds of Formula VII by addition of an organometallic reagent of Formula VIII, wherein M is Li, MgCl, MgBr or MgI:

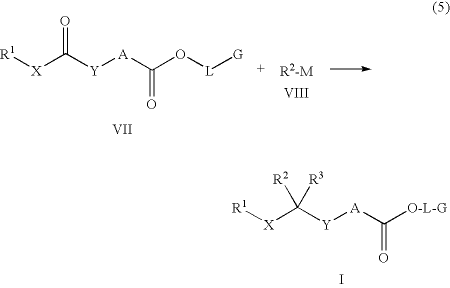

In the sixth process, optionally protected compounds of Formula I, wherein R³ is —OH, are prepared from ketone compounds of Formula IX by addition of an organometallic reagent of Formula X, wherein M is Li, MgCl, MgBr or MgI:

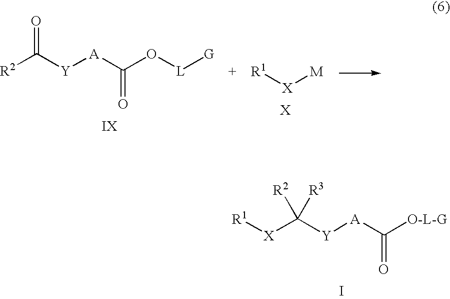

In the seventh process, a compound of Formula I, wherein R² is attached to the molecule through an ether linkage and R³ is —H, is prepared by reaction of an alcohol intermediate of Formula XI with an alkylating agent of Formula XII, wherein Z² is halide, preferably bromide or iodide, alkanesulfonate, haloalkanesulfonate or arylsulfonate:

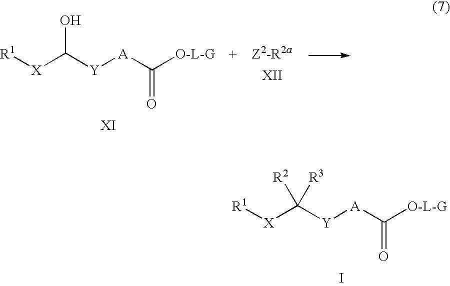

In the eighth process, a compound of Formula I, wherein R² is attached to the molecule through an ether linkage, R³ is —H, X and Y are bonds, and both R¹ and A are aromatic or heteroaromatic rings, is prepared by reaction of an alcohol intermediate of Formula XIII with a second alcohol of Formula XIV under acidic conditions:

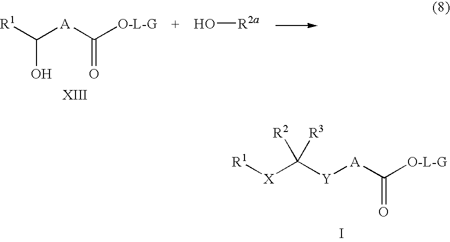

In the ninth process, compounds of Formula I can be prepared from other compounds of Formula I and protected compounds of Formula I:

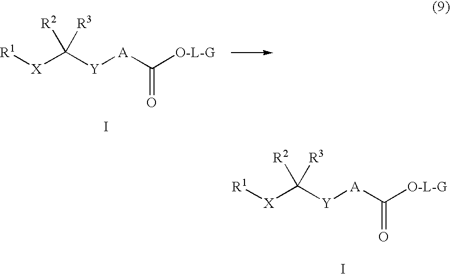

(1) when R¹ is bromophenyl or iodophenyl, it may be transformed into a compound in which R¹ is biphenyl by palladium catalyzed coupling with a phenylboronic acid under Suzuki conditions;

(2) when R¹ is bromophenyl or iodophenyl, it may be transformed a compound in which R¹ is alkynylphenyl by palladium catalyzed coupling with a terminal alkyne under Sonogashira conditions;

(3) when R¹ is bromophenyl or iodophenyl, it may be transformed into a compound in which R¹ is allylphenyl by palladium catalyzed coupling with tetraallyltin using a Stille conditions;

(4) when R¹ is bromophenyl or iodophenyl, it may be transformed into a compound in which R¹ is cyanophenyl using CuCN;

(5) when R¹ is hydroxyphenyl, it may be alkylated with an alkyl halide, cycloalkyl halide or cycloalkylalkyl halide in the presence of a base such as sodium hydride to yield a compound in which R¹ is alkoxyphenyl, cycloalkoxyphenyl or cycloalkylalkoxyphenyl;

(6) when R² is alkenyl or alkenyloxy, it may be transformed into a compound in which R² is hydroxyalkyl or hydroxyalkoxy by hydroboration;

(7) when R² is hydroxyalkyl, it may be transformed into a compound in which R² is alkoxycarbonylaminoalkyl by the following steps: a) conversion of the hydroxyl to the corresponding methanesulfonate; b) displacement of the methanesulfonate by azide anion; c) reduction of the azide; and d) acylation with an alkyl chloroformate; (8) when R³ is —OH, it may be transformed into a compound in which R³ is H by direct deoxygenation with Raney nickel, or by dehydration followed by hydrogenation;

(9) when R³ is —OH, it may be transformed into a compound in which R³ is alkanoylamino by treatment with an alkyl nitrile in the presence of strong acid (Ritter reaction).

Intermediates of Formula II, wherein Z¹=chlorine and the carbonyl carbon is attached to carbon atom that is part of ring A, are prepared from carboxylic acid intermediates of Formula XV:

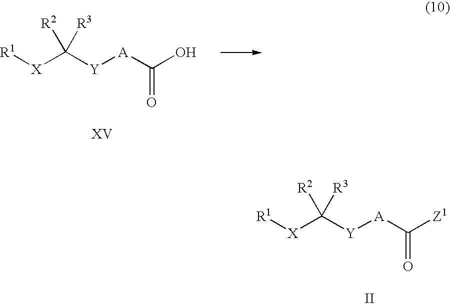

by reaction with, for example, thionyl chloride, oxalyl chloride, or phosphorus oxychloride.

Intermediates of Formula II, wherein the carbonyl carbon is attached to a nitrogen atom that is part of ring A, and Z¹ is chlorine, 1-imidazolyl, or p-nitrophenoxy, are prepared from intermediates of Formula IV, wherein H is attached to a nitrogen atom that is part of ring A, by reaction with phosgene, 1,1′-carbonyldiimidazole, or p-nitrophenyl chloroformate, respectively:

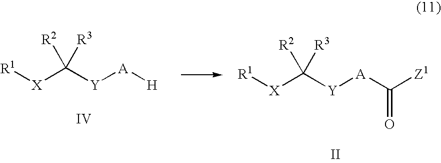

Intermediates of Formula IV, wherein H is attached to a nitrogen atom that is part of A, are prepared from intermediates of Formula XVI:

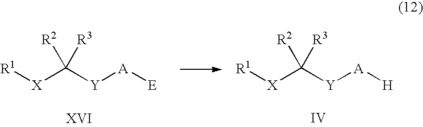

wherein E is an amine protecting group, including carbamate, amide, and sulfonamide protecting groups known in the art (T. W. GREENE & P. G. M. WUTS, PROTECTIVE GROUPS IN ORGANIC SYNTHESIS, John Wiley & Sons, Inc., New York 1999).

Intermediates of Formula XVI, wherein R³ is —OH, are prepared from ketone intermediates of Formula XVII by addition of an organometallic reagent of Formula VII, wherein M is, for example, Li, MgCl, MgBr, or MgI:

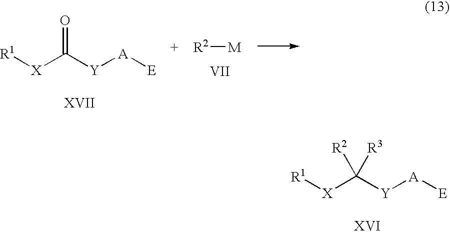

Intermediates of Formula XVI, wherein R² is a group attached by an ether linkage, are prepared from alcohol intermediates of Formula XVIII by reaction under basic conditions with alkylating agents of Formula XII, wherein Z² is a leaving group such as halide, alkanesulfonate, haloalkanesulfonate or arylsulfonate:

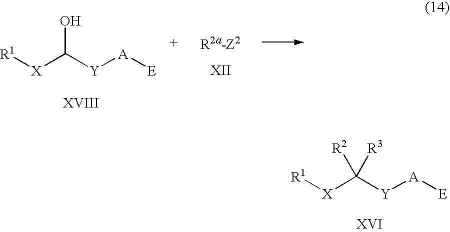

or by reaction with an alcohol of formula R^{2a}OH under acidic conditions.

Alcohol intermediates of Formula XVIII are prepared by reduction of ketone intermediates of Formula XVII with, for example, a hydride reducing agent such NaBFI₄, LiAlH₄ or diisobutylaluminum hydride:

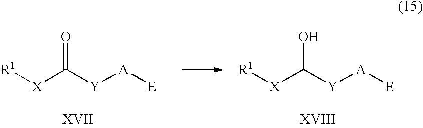

or by addition of an organometallic reagent of Formula X, wherein M is, for example, Li, MgCl, MgBr, or MgI to an aldehyde of Formula XIX:

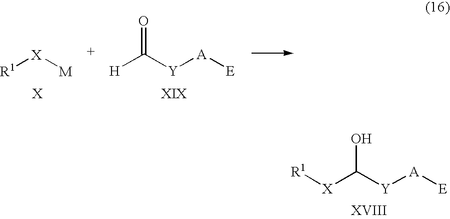

Ketone intermediates of Formula XVII are prepared by the addition of an organometallic reagent of Formula X to a carboxylic acid derivative of Formula XX, wherein Z³ is an alkoxide, dialkylamino group, or preferably an N-alkoxy-N-alkylamino group:

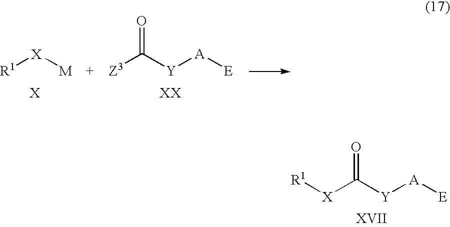

Organometallic reagents of Formula X are prepared by known process including halogen-lithium exchange, ortho-lithiation and treatment of halides R¹—X-Hal with magnesium or lithium metal.

Aldehyde intermediates of Formula XIX are prepared by reduction of carboxylic acid derivatives of Formula XX, wherein Z³ is an alkoxy or an N-alkoxy-N-alkylamino group, by using, for example, a hydride reducing agent such as LiAlH₄ or diisobutylaluminum hydride:

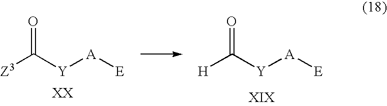

Ketone intermediates of Formula XVII are also prepared by oxidation of alcohol intermediates of Formula XVIII:

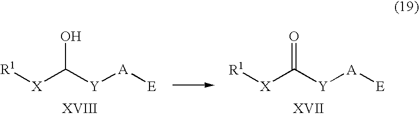

Alcohol intermediates of Formula III, wherein L is a linear C₂ alkyl chain substituted by one group R⁵ and G is —NHR⁹ or —NR⁹R¹⁰, are prepared by reaction of epoxide intermediates of Formula XXI with primary amines R⁹NH₂ or secondary amines R⁹NHR¹⁰:

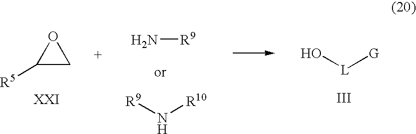

Alcohol intermediates of Formula III, wherein L is a linear C₂ alkyl chain substituted by one group R⁵ attached to the alcohol bearing carbon and G is —NH₂, are prepared by reaction of epoxide intermediates of Formula XXI with, for example NaN₃ in warm DMF, followed by reduction of azido intermediate of Formula XXII using catalytic hydrogenation or PPh₃ in wet THF:

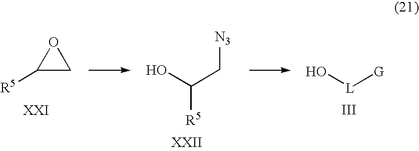

Epoxides of Formula XXI are prepared from alkenes of Formula XXIII by reaction with peracids such as m-chloroperoxybenzoic acid:

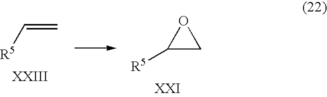

Alkenes of Formula XXIII are prepared from aldehydes of Formula XXIV by treatment with Tebbe reagent or triphenylphosphonium methylide:

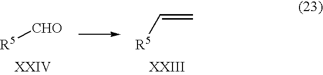

Epoxides of Formula XXI are also prepared by the reaction of aldehydes of Formula XXIV with trimethylsulfoxonium iodide or trimethylsulfonium iodide (J. Aube, Epoxidation and Related Processes, in 1 COMPREHENSIVE ORGANIC SYNTHESIS ch. 3.2 (B. M. Trost et al., eds., Pergamon Press New York, 1992):

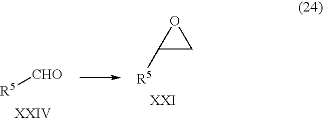

Alcohol intermediates of Formula III, wherein L is a linear C₂ alkyl chain substituted by one group R⁵ attached to the alcohol bearing carbon and G is —NH₂, —NHR⁹ or —NR⁹R¹⁰, are prepared by reduction of amides of α-hydroxyamide intermediates of Formula XXV, wherein R^{x}═R^{y}═H, R^{x}═R⁹ and R^{y}═H, or R^{x}═R⁹ and R^{y}═R¹⁰, with, for example, BH₃.THF or LiAlH₄:

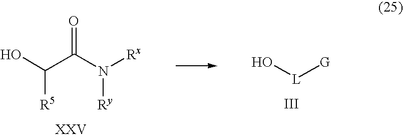

α-Hydroxyamide intermediates of Formula XXV are prepared by coupling α-hydroxyacid intermediates of Formula XXVI with amines of Formula XXVII using, for example, EDC in the presence of HOBt:

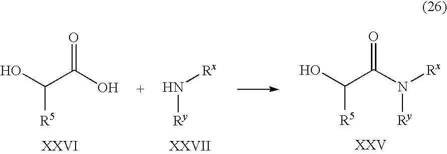

α-Hydroxyacid intermediates of Formula XXVI are prepared by diazotization of α-aminoacids of Formula XXVIII:

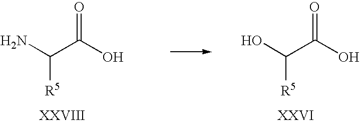

Alternatively, α-hydroxyacid intermediates of Formula XXVI are prepared by hydrolysis of cyanohydrin intermediates of Formula XXIX:

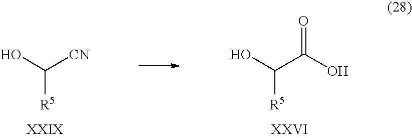

Alcohol intermediates of Formula III, wherein L is a linear C₂ alkyl chain substituted by one group R⁵ attached to the nitrogen bearing carbon and G is —NH₂, are prepared by reduction α-aminoacids of Formula XXVIII using, for example, BH₃.THF or LiAlH₄:

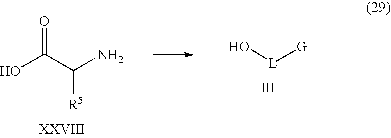

Intermediates of Formula V, wherein Z¹ is chlorine, 1-imidazolyl, or p-nitrophenoxy, are prepared from alcohol intermediates of Formula III by reaction with phosgene, 1,1′-carbonyldiimidazole, or p-nitrophenyl chloroformate:

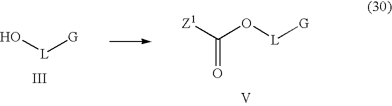

Aldehyde intermediates of Formula VI are prepared by oxidation of primary alcohol intermediates of Formula XXX by oxidation with, for example, Dess-Martin periodinane reagent or pyridinum chlorochromate:

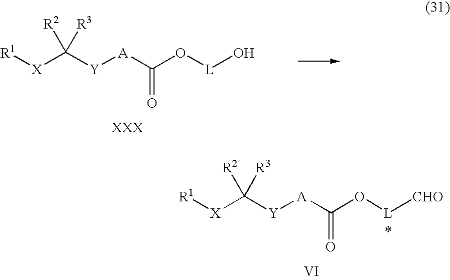

Primary alcohol intermediates of Formula XXX are prepared from diol intermediates HO-L-OH by routes analogous to those shown in equations 1 and 2.

The invention is further defined by reference to the examples, which are intended to be illustrative and not limiting.

Representative compounds of the invention can be synthesized in accordance with the general synthetic schemes described above and are illustrated in the examples that follow. The methods for preparing the various starting materials used in the schemes and examples are well within the knowledge of persons skilled in the art.

The following abbreviations have the indicated meanings:

| Abbreviation | Meaning |
|---|---|
| AcOH | acetic acid |
| aq | aqueous |
| Boc | tert-butoxy carbonyl or t-butoxy carbonyl |
| BF₃•Et₂O | boron trifluoride etherate |
| (Boc)₂O | di-tert-butyl dicarbonate |
| brine | saturated aqueous NaCl |
| CH₂Cl₂ | methylene chloride |
| CH₃CN | acetonitrile |
| Cpd | compound |
| d | day |
| DIBAL-H | diisobutylaluminum hydride |
| DIEA | N,N-diisopropylethylamine |
| DMAP | 4-(dimethylamino)pyridine |
| DMF | N,N-dimethylformamide |
| equiv | equivalents |
| Et | ethyl |
| EtOAc | ethyl acetate |
| h, hr | hour |
| H₂ | hydrogen gas |
| HOBt | 1-hydroxybenzotriazole |
| HBTU | 2-(1H-Benzotriazole-1-yl)-1,1,3,3- |
| | tetramethyluronium hexafluorophosphate |
| HCl | hydrochloric acid |
| HPLC | High Performance Liquid Chromatography |
| K₂CO₃ | potassium carbonate |
| KHSO₄ | potassium bisulfate |
| LC-MS | liquid chromatography-mass spectroscopy |
| LiCl | lithium chloride |
| m-CPBA | meta-chloroperoxybenzoic acid |
| Me | methyl |
| MeCN | acetonitrile |
| MeOH | methanol |
| min | minute |
| MS | mass spectrum |
| N₂ | nitrogen gas |
| NaBH₄ | sodium borohydride |
| NaBr | sodium bromide |
| NaHCO₃ | sodium bicarbonate |
| NaOH | sodium hydroxide |
| Na₂SO₄ | sodium sulfate |
| NH₃ | ammonia |
| NH₄Cl | ammonium chloride |
| Pd-C | palladium on carbon |
| PtO₂ | platinum (IV) oxide |
| p-TSA | para-toluenesulfonic acid |
| Red-Al | Sodium bis(2-methoxyethoxy)aluminum |
| | dihydride |
| rt | room temperature |
| satd | saturated |
| TEA | triethylamine or Et₃N |
| TEMPO | 2,2,6,6-tetramethyl-1-piperidinyloxy, |
| | free radical |
| TFA | trifluoroacetic acid |
| THF | tetrahydrofuran |
| TiCl₄ | titanium (IV) chloride |
| TiCl(O-i-Pr)₃ | chloride titanium(IV) triisopropoxide |
| TLC | thin layer chromatography |
| t_{R} | retention time |
| TsCl | tosyl chloride |

### Purification Methods

Preparative HPLC refers to reverse phase HPLC on a C-18 column eluted with a water/acetonitrile gradient containing 0.01% TFA run on a Gilson 215 system.

Column chromatography and flash chromatography refer to normal phase chromatography on a silica gel column or cartridge eluted with an hexanes/EtOAc gradient.

### Analytical Methods

LC-MS (3 min)
LC Conditions:
Column: Chromolith SpeedRod, RP-18e, 50×4.6 mm; Mobil phase: A: 0.01% TFA/water, B: 0.01% TFA/CH₃CN; Flow rate: 1 mL/min; Gradient:

| Time (min) | A % | B % |
|---|---|---|
| 0.0 | 90 | 10 |
| 2.0 | 10 | 90 |
| 2.4 | 10 | 90 |
| 2.5 | 90 | 10 |
| 3.0 | 90 | 10 |

MS Conditions: Electrospray ionization.

### Synthetic Procedures

### Preparation 1

### (S)-1-(3-chloro-2-fluorophenyl)-5-methoxy-1-((R)-piperidin-3-yl)pentan-1-ol

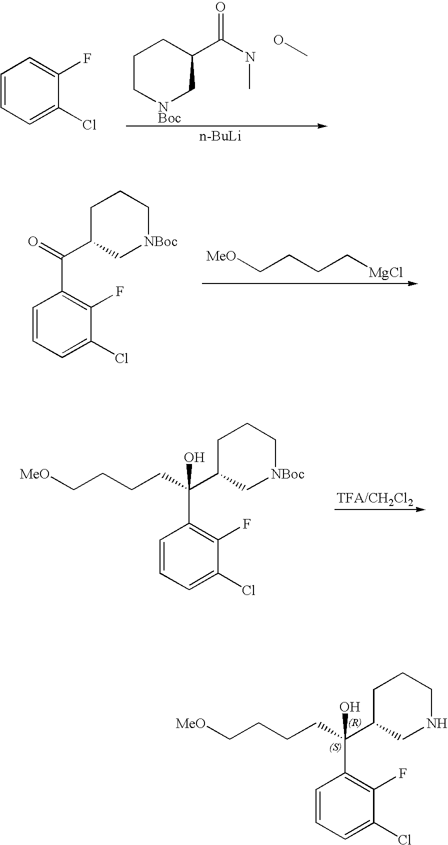

### Step 1. (R)-tert-butyl 3-(3-chloro-2-fluorobenzoyl)piperidine-1-carboxylate

2.5 M BuLi in hexane (40 mL, 0.1 mol) was added dropwise over 45 min to a stirred solution of 1-chloro-2-fluoro-benzene (13.0 g, 0.1 mol) in THF (250 mL) at −75° C. After additional stirring for 30 min at −75° C., a solution of (R)-tert-butyl 3-(methoxy(methyl)carbamoyl)-piperidine-1-carboxylate (21.76 g, 0.08 mol) in THF (100 mL) was added dropwise over 30 min. The mixture was allowed to warm to 0° C. The mixture was quenched with sat'd aq. NH₄Cl, extracted with EtOAc (3×) and the combined organic layers were dried over Na₂SO₄. Solvent removal and flash column chromatography, eluting with 5% EtOAc/PE, afforded (R)-tert-butyl 3-(3-chloro-2-fluorobenzoyl)piperidine-1-carboxylate (19.2 g, 70%). ¹H NMR (400 MHz, CDCl₃): δ=1.45 (s, 9H), 1.63 (m, 2H), 1.76 (m, 1H), 2.06 (m, 1H), 2.87(m, 1H), 3.15(m, 1H), 3.25 (m, 1H), 3.9 (m, 1H), 4.2 (m, 1H), 7.18 (m, 1H), 7.60 (m, 2H). MS (E/Z): 342 (M+H⁺).

### Step 2. (R)-tert-butyl 3-((S)-1-(3-chloro-2-fluorophenyl)-1-hydroxy-5-methoxypentyl)piperidine-1-carboxylate

A flame dried 250 mL three-necked flask was charged with magnesium turnings (7.02 g, 0.293 mol), a small crystal of iodine and THF (30 mL). The flask was evacuated and refilled with N₂. A solution of 1-chloro-4-methoxybutane (28.69 g, 0.234 mol) in THF (120 mL) was slowly added dropwise to the mixture. The reaction mixture was stirred at reflux for 2.5 h. and most of magnesium was consumed. The resulting Grignard reagent was used immediately. To another 100 mL three-necked flask was added (R)-tert-butyl 3-(3-chloro-2-fluorobenzoyl)-piperidine-1-carboxylate (10 g, 0.0293 mol) and THF (100 mL). The flask was evacuated and refilled with N₂. The mixture was cooled in a dry ice-acetone bath and the Grignard reagent (250 mL) was added. The reaction mixture was allowed to warm slowly to rt with stirring overnight. The mixture was quenched with satd aq NH₄Cl (50 mL), extracted with EtOAc (3×), and the combined organic layers were dried over Na₂SO₄. Evaporation of the solvent gave the crude product. LC-MS analysis of the crude product indicated the presence of two isomers (95:5). Flash column chromatography, eluting with 10% EtOAc/PE afforded (R)-tert-butyl 3-((S)-1-(3-chloro-2-fluorophenyl)-1-hydroxy-5-methoxypentyl)piperidine-1-carboxylate (9.4 g, 75% yield). ¹H NMR (400 MHz, DMSO): δ=0.68 (m, 1H), 1.50-1.01(m, 7H), 1.37 (s, 9H), 1.75 (m, 2H), 2.01 (m, 1H), 3.11 (s, 3H), 3.17(m, 2H), 3.85 (m, 1H), 7.2 (t, 1H), 7.45 (m, 2H). MS (E/Z): 430 (M+H⁺).

### Step 3. (S)-1-(3-chloro-2-fluorophenyl)-5-methoxy-1-((R)-piperidin-3-yl)pentan-1-ol

A solution of (R)-tert-butyl 3-((S)-1-(3-chloro-2-fluorophenyl)-1-hydroxy-5-methoxypentyl)piperidine-1-carboxylate (100 mg) in 20% TFA/CH₂Cl₂ was stirred at 0° C. for 30 min. The mixture was neutralized by addition of satd aq NaHCO₃, extracted with CH₂Cl₂ (3×) and dried over Na₂SO₄. Evaporation of the solvent gave (S)-1-(3-chloro-2-fluorophenyl)-5-methoxy-1-((R)-piperidin-3-yl)pentan-1-ol (70 mg, 91%), which was used without further purification. ¹H NMR (400 MHz, CDCl₃): δ=0.90(m, 1H), 1.52-1.24 (m, 6H), 1.78 (m, 1H), 1.83 (m, 1H), 1.93 (m, 1H), 2.21 (m, 1H), 2.40 (m, 1H), 2.83 (m, 1H), 3.00 (m, 1H), 3.12 (s, 3H), 3.31 (m, 2H), 3.63 (m, 1H), 7.06 (m, 1H), 7.30(m, 1H), 7.55 (t, 1H). MS (E/Z): 330 (M+H⁺).

### Preparation 2

### Methyl (S)-4-(3-chlorophenyl)-4-hydroxy-4-((R)-piperidin-3-yl)butylcarbamate

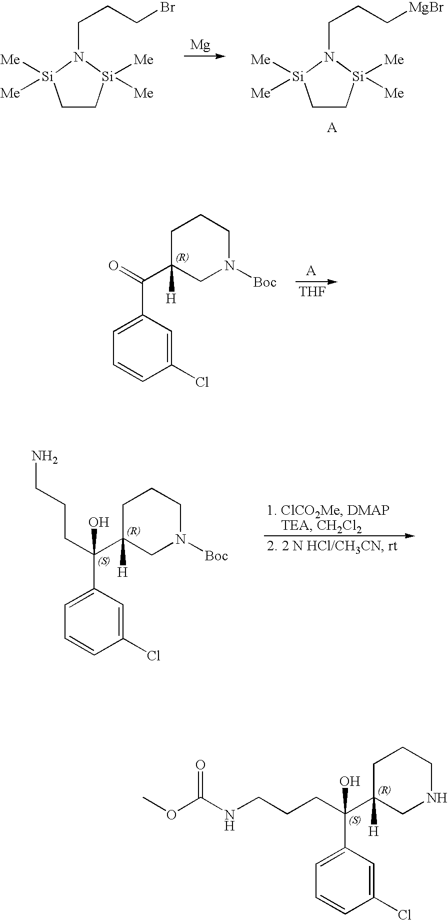

### Step 1. [3-(2,2,5,5-tetramethyl-1-aza-2,5-disilacyclopent-1-yl)propyl]magnesium bromide

A 250 mL round bottom flask was charged with magnesium turnings (0.528 g, 21.7 mmol, 1.16 equiv) and THF (10 mL). The flask was degassed and heated to 100° C. A small crystal of iodine was then added. A solution of 1-(3-bromopropyl)-2,2,5,5-tetramethyl-1-aza-2,5-disilacyclopentane (5.239 g, 18.7 mmol, 1.0 equiv) in THF (15 mL) was added dropwise to the boiling THF mixture over 10 min. The reaction mixture was stirred and heated under reflux for 2.5 h and most of magnesium was consumed. The resulting Grignard reagent (A) was used in the next step.

### Step 2. (R)-tert-butyl 3-((S)-4-amino-1-(3-chlorophenyl)-1-hydroxybutyl)piperidine-1-carboxylate

To a 250 mL, round bottom flask were added (3-chlorophenyl)((R)-N-Boc-piperidin-3-yl)methanone (0.800 g, 2.47 mmol) and THF (10 mL). The flask was evacuated and refilled with N₂. The mixture was cooled with a dry ice-acetone bath and the [3-(2,2,5,5-tetramethyl-1-aza-2,5-disilacyclopent-1-yl)propyl]magnesium bromide solution (A), obtained in Step 1, was added via a cannula. The reaction mixture was allowed to slowly warm to −8° C. while stirring overnight. The mixture was quenched with 10% aq Na₂CO₃ (10 mL), stirred at rt for 3 h, extracted with CH₂Cl₂ (3×), and dried over Na₂SO₄. The crude product was purified by reversed-phase HPLC (Phenomenex® Luna 5μ C18(2) 100 A, 250×21.20 mm, 5 micron, 10%→90% CH₃CN/H₂O, 0.1% CF₃COOH over 13 min and then 90% CH₃CN/H₂O, 0.1% CF₃COOH over 3.5 min, flow rate 25 mL/min) to give 0.883 g (72%) of TFA salt of (R)-tert-butyl 3-((S)-4-amino-1-(3-chlorophenyl)-1-hydroxybutyl)piperidine-1-carboxylate. LC-MS (3 min) t_{R}=1.30 min, m/z 383, 385 (MH⁺), 327, 329; ¹H NMR (400 MHz, CD₃OD) δ=7.36 (m, 1H), 7.27-7.13 (m, 3H), 4.26 (br s, 1H), 3.89 (d, J=12.9 Hz, 1H), 2.82-2.68 (m, 2H), 2.44 (br s, 1H), 2.36 (t, J=12.2 Hz, 1H), 1.97-1.79 (m, 2H), 1.64-1.08 (m, 16H), 1.34 (s); ¹³C NMR (100 MHz, CD₃OD) δ=156.69, 148.15, 135.39, 130.69, 127.74, 127.36, 125.41, 81.04, 78.10, 40.95, 28.69, 26.64, 26.51, 23.30.

### Step 3. (R)-tert-butyl 3-((S)-4-(methoxycarbonylamino)-1-(3-chlorophenyl)-1-hydroxybutyl)-piperidine-1-carboxylate

To a 100 mL round bottom flask were added the TFA salt of (R)-tert-butyl 3-((S)-4-amino-1-(3-chlorophenyl)-1-hydroxybutyl)piperidine-1-carboxylate (0.8164 g, 1.64 mmol, 1.0 equiv), DMAP (0.542 g), CH₂Cl₂ (40 mL) and triethylamine (6 mL). The mixture was cooled in an ice bath and a solution of methyl chloroformate (0.550 g, 5.82 mmol, 3.5 equiv) in CH₂Cl₂ (10 mL) was added. The reaction mixture was allowed to slowly warm to rt and stirred overnight. After the solvents were removed in vacuo, the residue was purified by reversed-phase HPLC (Phenomenex® Luna 5μ C18(2) 100 A, 250×21.20 mm, 5 micron, 70%→90% CH₃CN/H₂O, 0.1% CF₃COOH over 8 min and then 90% CH₃CN/H₂O, 0.1% CF₃COOH over 1.5 min, flow rate 25 mL/min) to give 0.5020 g (69%) of (R)-tert-butyl 3-((S)-4-(methoxycarbonyl-amino)-1-(3-chlorophenyl)-1-hydroxybutyl)piperidine-1-carboxylate. LC-MS (3 min) t_{R}=1.91 min, m/z 463 (MNa⁺), 441 (MH⁺), 343 341; ¹H NMR (400 MHz, CDCl₃) δ=7.37-7.36 (m, 1H), 7.28-7.17 (m, 3H), 4.90 (br s, 2H), 4.37 (d, J=12.0 Hz, 1H), 3.97 (d, J=12.3 Hz, 1H), 3.64 (s, 3H), 3.16-3.04 (m, 2H), 2.58-2.49 (m, 2H), 1.98-1.86 (m, 2), 1.76-1.70 (m, 1H), 1.61-1.56 (m, 1H), 1.45 (s, 9H), 1.48-1.13 (m, 5H); ¹³C NMR (100 MHz, CDCl₃) δ=157.60, 155.31, 146.51, 134.31, 129.36, 126.72, 125.96, 123.76, 80.08, 77.65, 52.21, 46.45, 44.91, 44.56, 40.91, 35.97, 28.42, 25.33, 25.25, 24.34.

### Step 4. Methyl (S)-4-(3-chlorophenyl)-4-hydroxy-4-((R)-piperidin-3-yl)butylcarbamate

A mixture of (R)-tert-butyl 3-((S)-4-(methoxycarbonylamino)-1-(3-chlorophenyl)-1-hydroxybutyl)piperidine-1-carboxylate (0.0322 g, 0.073 mmol), obtained as described above, in CH₃CN (30 mL) and 2 N aq HCl (25 mL) was vigorously stirred at rt for 24 h. The solvents were removed in vacuo to give the HCl salt of methyl (S)-4-(3-chlorophenyl)-4-hydroxy-4-((R)-piperidin-3-yl)butylcarbamate, which was used without further purification. LC-MS (3 min) t_{R}=0.98 min, m/z 343, 341 (M+H⁺), 323.

### Preparation 3

### (R)-1-(3-Chloro-2-fluorophenyl)-5-methoxy-1-((R)-morpholin-2-yl)pentan-1-ol

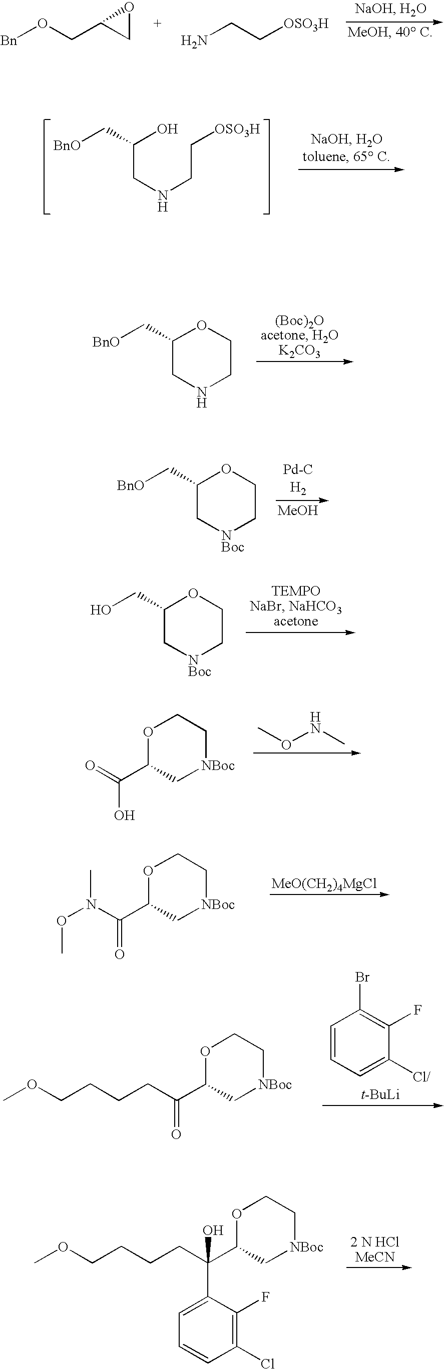

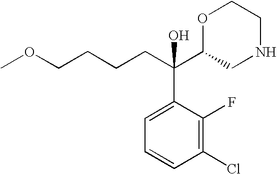

### Step 1. (R)-2-(Benzyloxymethyl)morpholine

2-Aminoethyl hydrogen sulfate (36.8 g, 255.8 mmol) was added in portions to a stirred mixture of (R)-2-(benzyloxymethyl)oxirane (10.0 g, 60.9 mmol) and NaOH (19.49 g, 487.2 mmol) in H₂O (46 mL) and MeOH (18 mL). After addition, the reaction mixture was stirred at 40° C. for 2 h. After cooling to rt, the mixture was treated with NaOH (15.0 g, 375.0 mmol), then toluene (70 mL) and stirred at 65° C. overnight. The mixture was cooled, diluted with toluene (27 mL) and H₂O (92 mL). The toluene layer was separated and the aqueous layer was extracted with CH₂Cl₂ (2×50 mL). The combined organic layers were concentrated to give crude (R)-2-(benzyloxymethyl)morpholine (˜14 g), which was used without purification. MS m/z 208 (M+H⁺).

### Step 2. (R)-tert-Butyl 2-(benzyloxymethyl)morpholine-4-carboxylate

To a solution of crude (R)-2-(benzyloxymethyl)morpholine (˜14 g) in acetone (100 mL) and H₂O (30 mL) at 0° C., was added K₂CO₃ (25.2 g, 182.7 mmol), followed by (Boc)₂O (14.6 g, 67.0 mmol). The resulting solution was warmed to rt, and stirred until no starting material remained (˜30 min). The acetone was removed under vacuum and the aqueous solution was extracted with CH₂Cl₂ (4×10 mL). The combined organic layers were washed with H₂O (10 mL) and the solvent was removed. The residue was purified by flash column chromatography to give (R)-tert-butyl 2-(benzyloxymethyl)morpholine-4-carboxylate (8.33 g, 44% over 2 steps). ¹H NMR (400 MHz, CDCl₃): δ=7.34 (m, 5H), 4.56 (s, 2H), 3.88 (d, 2H), 3.82 (br, 1H), 3.40 (m, 1H), 3.48 (m, 3H), 2.94 (m, 1H), 2.76 (m, 1H), 1.44 (s, 9H); MS m/z 330 (M+Na⁺).

### Step 3. (R)-tert-Butyl 2-(hydroxymethyl)morpholine-4-carboxylate

To a solution of (R)-tert-butyl 2-(benzyloxymethyl)morpholine-4-carboxylate (8.33 g, 27.1 mmol) in EtOH was added Pd—C (wet, 3.6 g), and the resulting mixture was stirred at rt under a H₂ atmosphere overnight. After filtration, the solvent was removed under vacuum and the residue was purified by flash column chromatography to give (R)-tert-butyl 2-(hydroxymethyl)morpholine-4-carboxylate (5.84 g, 99%) as a clear oil. ¹H NMR (400 MHz, CDCl₃): δ=3.88 (d, 2H), 3.82 (br, 1H), 3.64 (d, 1H), 3.56 (m, 3H), 2.94 (m, 1H), 2.76 (m, 1H), 1.90 (br, 1H), 1.44 (s, 9H); MS m/z 218 (M+H⁺).

### Step 4. (R)-4-(tert-Butoxycarbonyl)morpholine-2-carboxylic acid

Satd aq NaHCO₃ (15 mL) was added to a solution of (R)-tert-butyl 2-(hydroxymethyl)-morpholine-4-carboxylate (1.09 g, 5.0 mmol) in acetone (50 mL), and stirred at 0° C. Solid NaBr (0.1 g, 1 mmol) and TEMPO (0.015 g, 0.1 mmol) were added. Trichloroisocyanuric acid (2.32 g, 10.0 mmol) was then added slowly over 20 min at 0° C. After addition, the mixture was warmed to rt and stirred overnight. 2-Propanol (3 mL) was added and the resulting solution was stirred at rt for 30 min, filtered through a pad of Celite, concentrated under vacuum, and treated with satd aq Na₂CO₃ (15 mL). The aqueous solution was washed with EtOAc (5 mL), acidified with 6 N HCl, and extracted with EtOAc (5×10 mL). These EtOAc extracts were combined, dried over Na₂SO₄ and concentrated to give (R)-4-(tert-butoxycarbonyl)morpholine-2-carboxylic acid (1.07 g, 92%) as a white solid. ¹H NMR (400 MHz, CDCl₃): δ=4.20 (br, 1H), 4.12 (d, 1H), 4.02 (d, 1H), 3.84 (m, 1H), 3.62 (m, 1H), 3.04 (m, 2H), 1.44 (s, 9H); MS m/z 232 (M+H⁺).

### Step 5. (R)-tert-Butyl 2-(methoxy(methyl)carbamoyl)morpholine-4-carboxylate

To a solution of (R)-4-(tert-butoxycarbonyl)morpholine-2-carboxylic acid (1.05 g, 4.54 mmol) in DMF (10 mL) at 0° C. were added N,O-dimethylhydroxylamine hydrochloride (1.36 g, 13.62 mmol), DIEA (3.9 mL, 22.7 mmol), HBTU (1.89 g, 4.99 mmol) and HOBt (0.67 g, 4.99 mmol). The resulting solution was warmed to rt and stirred until no starting material remained (˜2 h). The mixture was diluted with H₂O (10 mL) and extracted with EtOAc (4×10 mL). The combined organic layers were washed with 1 N aq HCl (10 mL), 1 N aq NaOH (3×10 mL), water (2×10 mL) and brine (10 mL), and dried over Na₂SO₄. The solvent was removed under vacuum to give (R)-t-butyl 2-(methoxy(methyl)carbamoyl)morpholine-4-carboxylate (1.40 g, quant.), which was used for the next step without further purification; ¹H NMR (400 MHz, CDCl₃) δ: 4.36 (br, 1H), 4.08 (m, 1H), 4.00 (d, 1H), 3.84 (m, 1H), 3.76 (s, 3H), 3.58 (m, 1H), 3.20 (s, 3H), 3.04 (m, 2H), 1.44 (s, 9H); MS m/z 297 (M+Na⁺).

### Step 6. (R)-tert-Butyl 2-(5-methoxypentanoyl)morpholine-4-carboxylate

(4-Methoxybutyl)magnesium chloride in THF (1.47 M, 10.2 mL, 15.0 mmol) was slowly added dropwise to a solution of (R)-tert-butyl 2-(methoxy(methyl)carbamoyl)morpholine-4-carboxylate (1.37 g, 5.0 mmol) in THF (10 mL) at −20° C., such that the temperature remained steady at around −20° C. After addition, the resulting solution was warmed to rt, and quenched with 1 N aq HCl (10 mL). The organic layer was separated, and the aqueous layer was extracted with ether (3×5 mL). The combined organic layers were washed with satd aq NaHCO₃ (10 mL) and brine (5 mL), and dried over Na₂SO₄. The solvent was then removed in vacuo to give (R)-tert-butyl 2-(5-methoxypentanoyl)morpholine-4-carboxylate (1.41 g, 93%), which was used for the next step without purification; MS m/s 324 (M+Na⁺).

### Step 7. (R)-tert-Butyl 2-((R)-1-(3-chloro-2-fluorophenyl)-1-hydroxy-5-methoxypentyl)-morpholine-4-carboxylate

t-BuLi in pentane (1.7 M, 7.96 mL, 13.5 mmol) was added dropwise to a solution of 1-bromo-3-chloro-2-fluorobenzene (1.42 g, 6.77 mmol) in THF (8 mL) at −70° C., such that the temperature remained at around −70° C. The resulting solution (A) was stirred at the same temperature for another 30 min, and used directly in the next step.

Solution (A) was added dropwise to a solution of (R)-tert-butyl 2-(5-methoxypentanoyl)morpholine-4-carboxylate (0.64 g, 2.12 mmol) in toluene (5 mL) at −20° C. The resulting solution was allowed to warm to rt slowly, and kept at same temperature for 1 h. The reaction was quenched with satd aq NH₄Cl (8 mL) and extracted with diethyl ether (4×10 mL). The combined organic layers were washed with water and brine, and solvent was removed in vacuo to give a crude product, which was purified by flash column chromatography to afford (R)-tert-butyl 2-((R)-1-(3-chloro-2-fluorophenyl)-1-hydroxy-5-methoxypentyl)morpholine-4-carboxylate (0.40 g, 43%). ¹H NMR (400 MHz, CDCl₃) δ: 7.44 (dd, 1H), 7.32 (dd, 1H), 7.04 (dd, 1H), 4.18 (br, 1H), 3.80 (m, 3H), 3.42 (dd, 1H), 3.24 (st, 5H), 3.04-2.80 (m, 3H), 2.04 (m, 1H), 1.68 (m, 1H), 1.44 (s, 9H), 1.30 (m, 3H), 0.86 (m, 1H); MS m/z 454 (M+Na⁺).

### Step 8. (R)-1-(3-Chloro-2-fluorophenyl)-5-methoxy-1-((R)-morpholin-2-yl)pentan-1-ol

To a solution of (R)-tert-butyl 2-((R)-1-(3-chloro-2-fluorophenyl)-1-hydroxy-5-methoxypentyl)morpholine-4-carboxylate (0.38 g, 0.88 mmol) in MeCN (50 mL), 2 N aq HCl (50 mL) was added slowly at rt. The resulting solution was stirred at rt overnight, basified to pH=10 with 10 N aq NaOH, and evaporated under reduced pressure to remove MeCN. The aqueous layer was extracted with CH₂Cl₂ (4×5 mL). The combined organic layers were washed with brine and dried over Na₂SO₄. The solvent was removed in vacuo to give (R)-1-(3-chloro-2-fluorophenyl)-5-methoxy-1-((R)-morpholin-2-yl)pentan-1-ol (0.27 g, 93%) as a free amine. The crude product was used for next step without purification; MS m/z 332 (M+H⁺).

### Preparation 4

### Methyl 2-((R)-(3-chlorophenyl)((R)-piperidin-3-yl)methoxy)ethylcarbamate

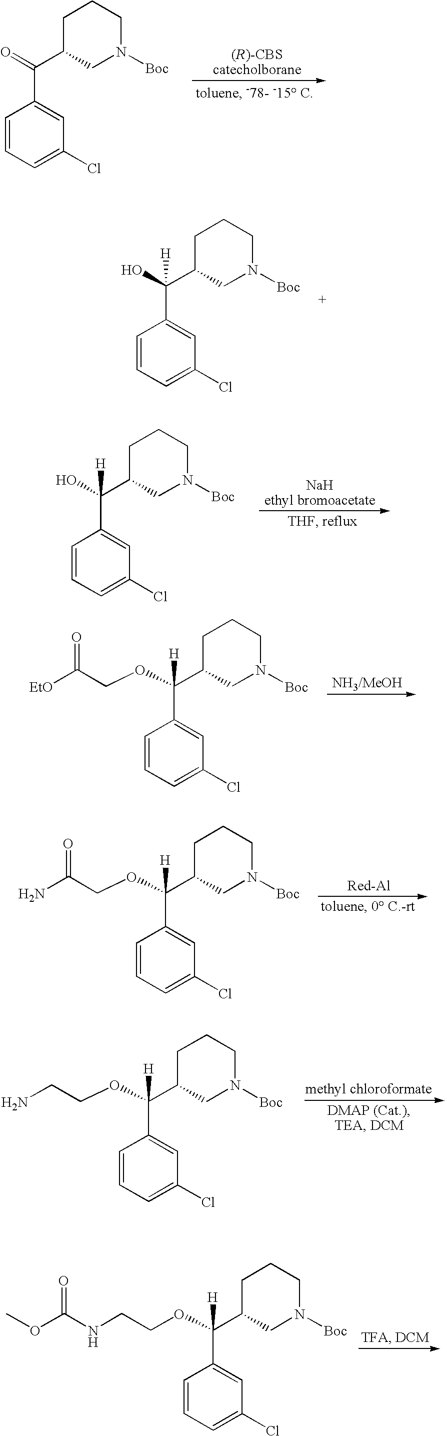

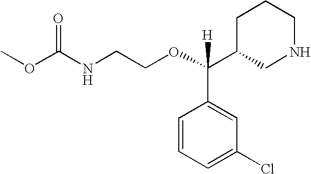

### Step 1: (R)-tert-Butyl 3-((R)-(3-chlorophenyl)(hydroxy)methyl)piperidine-1-carboxylate

Catecholborane (5.6 mL, 54.0 mmol) was added dropwise to a solution of (R)-2-methyl-CBS-oxazaborolidine (1 M in toluene, 9 mL, 9.00 mmol) and (R)-tert-butyl 3-(3-chlorobenzoyl)piperidine-1-carboxylate (5.60 g, 17.29 mmol) that was cooled to −78° C. After 20 min, the reaction temperature was allowed to warm to −15° C. and stirred overnight. The reaction was quenched at 0° C. by careful addition of water and diluted with ether. The resulting suspension was filtered through Celite and washed with ether. The filtrate was washed successively with 1 M aq NaOH (3×50 mL), 1 M aq HCl (3×50 mL), satd aq NaHCO₃ and brine, and dried over Na₂SO₄. The solution was filtered, the filtrate was evaporated under vacuum, and the residue was purified by preparative HPLC to afford (R)-tert-butyl 3-((R)-(3-chlorophenyl)(hydroxy)methyl)piperidine-1-carboxylate (2.44 g) and (R)-tert-butyl 3-((S)-(3-chlorophenyl)(hydroxy)methyl)piperidine-1-carboxylate (1.21 g). MS: 348 (M+Na)⁺.

### Step 2: (R)-tert-Butyl 3-((R)-(3-chlorophenyl)(2-ethoxy-2-oxoethoxy)methyl)piperidine-1-carboxylate

To a suspension of 60% NaH in oil (960 mg, 24.0 mmol) and anhydrous THF at 0° C. was added a solution of (R)-tert-butyl 3-((R)-(3-chlorophenyl)(2-ethoxy-2-oxoethoxy)methyl)-piperidine-1-carboxylate (1.429 g, 4.40 mmol) in anhydrous THF (10 mL). The reaction mixture was stirred at rt for 30 min and a solution of ethyl bromoacetate (2.204 g, 13.2 mmol) in anhydrous THF (10 mL) was added dropwise. The resulting suspension was heated at reflux for 3 h and cooled to 0° C. again. A suspension of NaH in oil (960 mg, 24.0 mmol) was added and stirred for 30 min at rt, followed by addition of a solution of ethyl bromoacetate (2.204 g, 13.2 mmol), and the mixture was heated at reflux overnight. The reaction mixture was cooled to 0° C. and quenched by careful addition of aq NH₄Cl. The mixture was extracted with EtOAc (3×). The combined organic phases were washed with brine, dried over Na₂SO₄, and filtered. The filtrate was evaporated and the residue was purified by flash chromatography on silica gel to afford (R)-tert-butyl 3-((R)-(3-chlorophenyl)(2-ethoxy-2-oxoethoxy)methyl)piperidine-1-carboxylate (1.62 g). MS: 412 (M+H)⁺.

### Step 3: (R)-tert-Butyl 3-((R)-(2-amino-2-oxoethoxy)(3-chlorophenyl)methyl)piperidine-1-carboxylate

(R)-tert-Butyl 3-((R)-(3-chlorophenyl)(2-ethoxy-2-oxoethoxy)methyl)piperidine-1-carboxylate (1.50 g, 3.65 mmol) was dissolved in 7 M NH₃ in MeOH, and stirred at rt for 6 h. The mixture was evaporated under reduced pressure to afford the (R)-tert-butyl 3-((R)-(2-amino-2-oxoethoxy)(3-chlorophenyl)methyl)piperidine-1-carboxylate in quantitative yield. MS: 383 (M+H)⁺.

### Step 4: (R)-tert-Butyl 3-((R)-(2-aminoethoxy)(3-chlorophenyl)methyl)piperidine-1-carboxylate

(R)-tert-Butyl 3-((R)-(2-amino-2-oxoethoxy)(3-chlorophenyl)methyl)piperidine-1-carboxylate (1.10 g, 2.60 mmol) was dissolved in anhydrous toluene (30 mL) and cooled to 0° C. Red-Al (65% in toluene, 2.6 mL, 8.64 mmol) was added dropwise. After the addition, the reaction was stirred at rt for 12 h and quenched by adding water slowly. The resulting mixture was filtered through Celite, washing with THF. The filtrate was evaporated under reduced pressure to give crude (R)-tert-butyl 3-((R)-(2-aminoethoxy)(3-chlorophenyl)methyl)piperidine-1-carboxylate (1.05 g), which was used for next step without further purification.

### Step 5: (R)-tert-Butyl 3-((R)-(3-chlorophenyl)(2-(methoxycarbonylamino)ethoxy)methyl)piperidine-1-carboxylate

To a solution of (R)-tert-butyl 3-((R)-(2-aminoethoxy)(3-chlorophenyl)methyl)piperidine-1-carboxylate (1.05 g, ca. 2.6 mmol), TEA (3.96 mL, 2.85 mmol), and DMAP (174 mg, 1.43 mmol) in anhydrous CH₂Cl₂ (20 mL) cooled to 0° C. was added a solution of methyl chloroformate (1.35 g, 14.25 mmol) in CH₂Cl₂ (20 mL) within 30 min. The reaction was stirred overnight, and evaporated under vacuum. The residue was purified by flash chromatography on silica gel to afford (R)-tert-butyl 3-((R)-(3-chlorophenyl)(2-(methoxycarbonylamino)ethoxy)methyl)piperidine-1-carboxylate (0.65 g). MS: 427 (M+H)⁺.

### Step 6: Methyl 2-((R)-(3-chlorophenyl)((R)-piperidin-3-yl)methoxy)ethylcarbamate

TFA (0.5 mL) was added to a stirred solution of (R)-tert-butyl 3-((R)-(3-chlorophenyl)(2-(methoxycarbonylamino)-ethoxy)methyl)piperidine-1-carboxylate (91 mg, 0.21 mmol) in CH₂Cl₂ (3 mL) at rt. The mixture was stirred until complete removal of the Boc group had occurred. The solvent was removed under vacuum to give methyl 2-((R)-(3-chlorophenyl)((R)-piperidin-3-yl)methoxy)ethylcarbamate as its TFA salt. MS: 327 (M+H)⁺.

### Preparation 5

### 2,2-dimethyl-4-(((R)-tetrahydro-2H-pyran-3-yl)methyl)oxazolidine

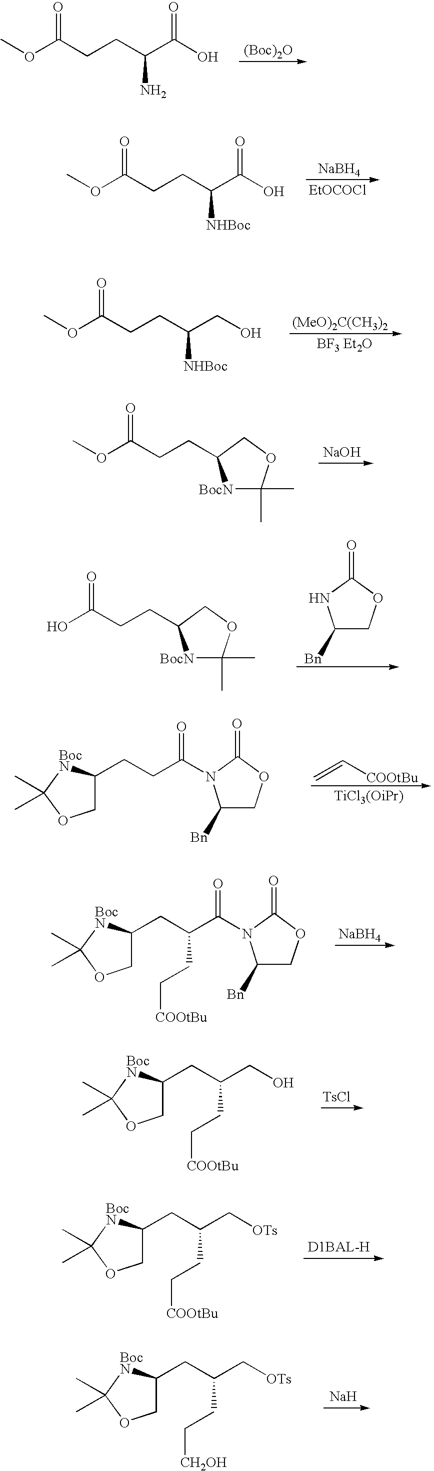

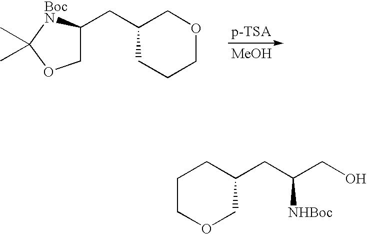

### Step 1. (S)-2-(tert-butoxycarbonylamino)-5-methoxy-5-oxopentanoic acid

In a round bottom flask, TEA (303 g, 3 mol) was added dropwise to a stirred solution of Boc₂O (261.6 g, 1.2 mol) and 2-amino-pentanedioic acid 5-methyl ester (161 g, 1 mol) in water (800 ml) and dioxane (800 ml). After 18 hr, the solution was extracted with petroleum ether (2×1000 ml) and the aqueous phase was cooled on ice and carefully acidified to pH 3 by slow addition of 10% citric acid solution. The urethane was then extracted into EtOAc (3×1000 ml) and the combined extracts were washed with brine, then dried (Na₂SO₄), filtered and concentrated under reduced pressure to give (S)-2-(tert-butoxycarbonylamino)-5-methoxy-5-oxopentanoic acid (238 g, 91.2%), which was used without further purification.

### Step 2. (5)-methyl 4-(tert-butoxycarbonylamino)-5-hydroxypentanoate

N-methylmorpholine (15 mL, 0.135 mol) was added to a stirred solution of (S)-2-(tert-butoxycarbonylamino)-5-methoxy-5-oxopentanoic acid (35.2 g, 0.135 mol) in THF (500 mL) at −10° C., followed by the addition of ethyl chloroformate (14.72 g, 0.135 mol). After 10 min, NaBH₄ (15.37 g, 0.405 mol) was added in one portion. MeOH (1200 mL) was then added dropwise to the mixture over a period of 20 min at 0° C. The solution was stirred for an additional 20 min and then neutralized with 1M KHSO₄. The organic solvent was removed and the aqueous layer was extracted with EtOAc (3×500 ml). The combined organic phases were washed consecutively with 1M KHSO₄ (300 mL), H₂O (300 mL), 5% aqueous NaHCO₃ (300 mL), and dried (Na₂SO₄). The solvent was evaporated to give a residue, which was purified by column chromatography to give the desired (S)-methyl 4-(tert-butoxycarbonylamino)-5-hydroxypentanoate (24 g, 72%).

### Step 3. (S)-tert-butyl 4-(3-methoxy-3-oxopropyl)-2,2-dimethyloxazolidine-3-carboxylate

(S)-methyl 4-(tert-butoxycarbonylamino)-5-hydroxypentanoate (24 g, 97.2 mmol) and isopropenyl methyl ether (88.8 g, 854.6 mmol) were dissolved in acetone (2000 mL) and BF₃.Et₂O (0.82 mL, 5.84 mmol) was added at room temperature. The mixture was stirred for 1 hr at room temperature. The reaction was quenched by addition of TEA (11.6 mL). The reaction solution was washed with aqueous saturated NaHCO₃ (200 mL) and evaporated, and (S)-tert-butyl 4-(3-methoxy-3-oxopropyl)-2,2-dimethyloxazolidine-3-carboxylate (25.1 g, 90%) was obtained as an oil, which was used in the next step without further purification.

### Step 4. (S)-3-(3-(tert-butoxycarbonyl)-2,2-dimethyloxazolidin-4-yl)propanoic acid

An aqueous solution of sodium hydroxide (195 mL, 4.0 M in H₂O, 0.261 mol, 3.0 eq) was added to a solution of (S)-tert-butyl 4-(3-methoxy-3-oxopropyl)-2,2-dimethyloxazolidine-3-carboxylate (25.1 g, 0.087 mol), and the resulting cloudy reaction mixture was stirred at 23° C. for 3.5 hr. The mixture was concentrated under reduced pressure to ˜50 mL volume and then was partitioned between 0.5 M HCl (360 ml) and EtOAc (2×360 ml). The combined organic layers were dried over Na₂SO₄ and were filtered. The filtrate was concentrated under reduced pressure to give (S)-3-(3-(tert-butoxycarbonyl)-2,2-dimethyloxazolidin-4-yl)propanoic acid (21.6 g, 91%), which was used without further purification.

### Step 5. (S)-tert-butyl 2,2-dimethyl-4-(3-((R)-4-methyl-2-oxooxazolidin-3-yl)-3-oxopropyl)oxazolidine-3-carboxylate

A 2000 mL flask was charged with (S)-3-(3-(tert-butoxycarbonyl)-2,2-dimethyloxazolidin-4-yl)propanoic acid (21.6 g, 79 mmol) and 750 mL of dry THF. The solution was cooled to 0° C., then triethylamine (23.94 g, 237 mmol, 3.0 equiv) and pivaloyl chloride (9.76 mL, 79 mmol, 1.0 equiv) were sequentially added. The solution was stirred for 4 hr at 0° C. After this time, (R)-4-benzyl-2-oxalozolidinone (13.26 g, 75.2 mmol, 0.95 equiv) and dried LiCl (3.68 g, 86.4 mmol, 1.1 equiv) were added and the reaction was allowed to stir for 13 hr while warming to rt. After this time, 560 mL of 0.5 M HCl was added and the layers were separated. The aqueous layer was extracted with EtOAc (3×370 mL), and the combined organic layers washed with 10% K₂CO₃ (2×370 mL), and brine (2×370 mL), then dried over Na₂SO₄, and evaporated. The crude material was purified by flash chromatography, eluting with 0-29% EtOAc in hexanes. This afforded 26.3 g (81%) of (S)-tert-butyl 2,2-dimethyl-4-(3-((R)-4-methyl-2-oxooxazolidin-3-yl)-3-oxopropyl)oxazolidine-3-carboxylate as a clear syrup.

### Step 6. (S)-tert-butyl 4-((R)-5-tert-butoxy-2-((R)-4-methyl-2-oxooxazolidine-3-carbonyl)-5-oxopentyl)-2,2-dimethyloxazolidine-3-carboxylate

At 0° C., 1.0M TiCl₄ in CH₂Cl₂ solution (8.55 mL, 0.7 eq) was diluted with CH₂Cl₂ (100 mL) followed by the addition of 1.0M TiCl(Oi-Pr)₃ in hexanes (4.28 mL, 0.35 eq) and stirred 5 min. Then, DIEA (2.87 mL, 1.35 eq) was added and stirred 15 min, followed by the addition of a solution of (S)-tert-butyl 2,2-dimethyl-4-(3-((R)-4-methyl-2-oxooxazolidin-3-yl)-3-oxopropyl)oxazolidine-3-carboxylate (5.28 g, 12.22 mmol) in CH₂Cl₂ (50 mL). The reaction mixture was stirred 1 hr at 0° C. To the solution, t-butylacrylate (2.22 mL, 1.25 eq) was added and the mixture was left stirring over 48 hr, while allowing to warm to rt. The mixture was concentrated, partitioned between EtOAc (300 mL) and 1% HCl solution (100 mL). The organic layer was washed with sat. NaHCO₃ solution (60 mL), brine (60 mL), dried over Na₂SO₄. After filtration and concentration, the residue was purified by ISCO (120 g column, 0-35% EtOAc in Hexanes gradient) to afford 4.12 g (60%) (S)-tert-butyl 4-((R)-5-tert-butoxy-2-((R)-4-methyl-2-oxooxazolidine-3-carbonyl)-5-oxopentyl)-2,2-dimethyloxazolidine-3-carboxylate as a yellowish solid. MS ESI +ve m/z 583 (M+Na).

### Step 7. (S)-tert-butyl 4-((R)-5-tert-butoxy-2-(hydroxymethyl)-5-oxopentyl)-2,2-dimethyloxazolidine-3-carboxylate

(S)-tert-butyl 4-((R)-5-tert-butoxy-2-((R)-4-methyl-2-oxooxazolidine-3-carbonyl)-5-oxopentyl)-2,2-dimethyloxazolidine-3-carboxylate (4.12 g, 7.36 mmol) was dissolved in 4:1 THF:MeOH (200 mL) and cooled to 0° C. Sodium borohydride (557 mg, 2 eq) was added slowly. After 10 min. at 0° C., the mixture was slowly warmed to rt, and stirred 2 hr. The mixture was concentrated, redissolved in EtOAc (300 mL), washed with 1% HCl solution (100 mL), brine (60 mL), and dried over Na₂SO₄. After filtration and concentration, the residue was purified by ISCO (40 g column, 10-65% EtOAc in Hexanes gradient, check TLC with Ninhydrin stain) to afford 2.86 g of (S)-tert-butyl 4-((R)-5-tert-butoxy-2-(hydroxymethyl)-5-oxopentyl)-2,2-dimethyloxazolidine-3-carboxylate as a white solid. MS ESI +m/v 410 (M+Na).

### Step 8. (S)-tert-butyl 4-((R)-5-tert-butoxy-5-oxo-2-(tosyloxymethyl)pentyl)-2,2-dimethyloxazolidine-3-carboxylate

To a solution of (S)-tert-butyl 4-((R)-5-tert-butoxy-2-(hydroxymethyl)-5-oxopentyl)-2,2-dimethyloxazolidine-3-carboxylate (244 mg, 0.63 mmol) in anhydrous CH₂Cl₂ (6 mL) was added pyridine (2 mL) and catalytic amount of DMAP, the solution was chilled to 0° C. Tosyl chloride (360 mg, 1.88 mmol) was added and stirred at rt overnight. The reaction mixture was diluted with ethyl acetate (40 mL) and washed with 1 N HCl (2×, 50 ml+20 ml), followed by H₂O, aq. NaHCO₃, brine, dried over Na₂SO₄, and filtered. After evaporation of solvent, the residue was purified on silica gel column, eluted with 0-20% ethyl acetate in hexane to afford (S)-tert-butyl 4-((R)-5-tert-butoxy-5-oxo-2-(tosyloxymethyl)pentyl)-2,2-dimethyloxazolidine-3-carboxylate (317 mg, yield 93%).

### Step 9. (S)-tert-butyl 4-((R)-5-hydroxy-2-(tosyloxymethyl)pentyl)-2,2-dimethyloxazolidine-3-carboxylate

DiBAl—H (1 M in hexane, 1.75 mL, 1.75 mmol) was added dropwise to a solution of (S)-tert-butyl 4-((R)-5-tert-butoxy-5-oxo-2-(tosyloxymethyl)pentyl)-2,2-dimethyloxazolidine-3-carboxylate (317 mg, 0.58 mmol) in anhydrous CH₂Cl₂ (8 mL) at −78° C. under N₂. After the addition, the reaction mixture was stirred for another 30 min. The reaction was quenched with MeOH (2 mL), followed by an addition of a 50% aq solution of Rochelle's salt and stirred 2 hr. The resulting solution was extracted with CH₂Cl₂ (3×20 mL), and the combined organic phases were concentrated and dissolved in THF/MeOH (10 mL, 4/1, v/v), and chilled to 0° C., followed by the addition of NaBH₄ (11 mg, 0.29 mmol) and stirred 0° C. for 30 min. The reaction was quenched by aqueous NH₄Cl, then extracted with ethyl acetate (3×20 mL). The combined organic phases were washed with H₂O, brine, and dried over Na₂SO₄, filtered, and concentrated to give crude product (S)-tert-butyl 4-((R)-5-hydroxy-2-(tosyloxymethyl)pentyl)-2,2-dimethyloxazolidine-3-carboxylate (255 mg, 92%), which was used without further purification.

### Step 10. (S)-tert-butyl 2,2-dimethyl-4-(((R)-tetrahydro-2H-pyran-3-yl)methyl)oxazolidine-3-carboxylate

NaH (43 mg, 1.08 mmol) was added to a solution of (S)-tert-butyl 4-((R)-5-hydroxy-2-(tosyloxymethyl)pentyl)-2,2-dimethyloxazolidine-3-carboxylate (254 mg, 0.54 mmol) in anhydrous DMF (8 mL) at 0° C. under N₂. After stirring at 0° C. for 1 hr, the reaction was quenched with aq. NH₄Cl and evaporated to dryness. The residue was dissolved in ethyl acetate and H₂O, the separated aqueous phase was extracted with ethyl acetate. The combined organic phases were washed with H₂O, brine, and dried over Na₂SO₄, filtered, and evaporated. The crude product was purified on silica gel column to afford (S)-tert-butyl 2,2-dimethyl-4-(((R)-tetrahydro-2H-pyran-3-yl)methyl)oxazolidine-3-carboxylate (136 mg, 84%).

### Step 11. Preparation of tert-butyl (S)-1-hydroxy-3-((R)-tetrahydro-2H-pyran-3-yl)propan-2-ylcarbamate

p-TSA (37 mg, 0.22 mmol) was added to a solution of (S)-tert-butyl 2,2-dimethyl-4-(((R)-tetrahydro-2H-pyran-3-yl)methyl)oxazolidine-3-carboxylate (643 mg, 2.15 mmol) in MeOH (10 mL), and stirred at room temperature for 12 hr. TEA (2 mL) was added, followed by Boc₂O (46 mg, 0.21 mmol) with stirring for another 30 min. The solvent was removed under reduced pressure to give the crude product tert-butyl (S)-1-hydroxy-3-((R)-tetrahydro-2H-pyran-3-yl)propan-2-ylcarbamate. It was used in the next step without further purification. MS ESI +ve m/z 260 (M+1).

### Preparation 6

### (3SR,4RS)-tert-butyl 3-hydroxy-4-isobutylpiperidine-1-carboxylate

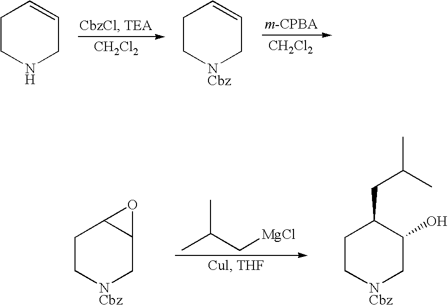

### Step 1. Benzyl 5,6-dihydropyridine-1(2H)-carboxylate

A solution of 1,2,3,6-tetrahydropyridine (5.0 g, 60.15 mmol) and triethylamine (16.77 mL, 2 equiv) in CH₂Cl₂ (50 mL) was cooled to 0° C. (ice/water bath), then benzyl chloroformate (9.7 mL, 1.1 equiv) was added slowly. After 30 min, the reaction mixture was allowed to warm slowly to rt and stirred for 4 h. LC-MS showed the reaction was complete. The mixture was diluted with ether (300 mL), washed with 5% aq HCl (2×50 mL), satd aq NaHCO₃ (40 mL), brine (40 mL), and dried over Na₂SO₄. After concentration, the desired product, benzyl 5,6-dihydropyridine-1(2H)-carboxylate (9.93 g, 78% yield), was left. LC-MS (3 min) t_{R}=1.74 min., m/z 218 (M+1).

### Step 2. Benzyl 7-oxa-3-azabicyclo[4.1.0]heptane-3-carboxylate

A solution of benzyl 5,6-dihydropyridine-1(2H)-carboxylate (9.93 g, 45.76 mmol) in CH₂Cl₂ (75 mL) was cooled to 0° C. (ice/water bath), and solid m-chloroperoxybenzoic acid (77%, 15.38 g, 1.5 equiv) was added. After 10 min at 0° C., the reaction mixture was warmed slowly to rt. After stirring for 2 h, LC-MS showed the reaction was complete. The mixture was diluted with ether (300 mL), washed with 5% aq NaOH (2×40 mL), 25% aq Na₂S₂O₃ solution (3×20 mL), brine (30 mL), and dried over Na₂SO₄. After concentration, the residue was purified by flash chromatography (120 g silica gel column, 12%-70% EtOAc in hexanes gradient, 2^{nd} UV peak) to afford benzyl 7-oxa-3-azabicyclo[4.1.0]heptane-3-carboxylate (7.87 g, 74% yield). LC-MS (3 min) t_{R}=1.41 min., m/z 234 (M+1).

### Step 3. (3S,4R)-benzyl 3-hydroxy-4-isobutylpiperidine-1-carboxylate

Benzyl 7-oxa-3-azabicyclo[4.1.0]heptane-3-carboxylate (90 mg, 0.386 mmol), CuI (15 mg, 0.2 equiv), under an atmosphere of N₂ gas (3×) was dissolved in dry THF (4 mL) and cooled to −40° C. 2.0 M Isobutylmagnesium bromide in ether (580 μL, 3 equiv) was added slowly. After 8 min, the reaction mixture was allowed warmed slowly to rt. After 20 min, the reaction mixture turned black. After stirring a further 2 h, LC-MS showed the reaction was complete. Satd aq NH₄Cl solution (4 mL) was added to quench the reaction. The reaction mixture was partitioned between EtOAc (40 mL) and satd aq NH₄Cl solution (15 mL). The aqueous layer was extracted with EtOAc (15 mL), then the combined EtOAc layers were washed with water (10 mL), brine (10 mL), and dried over Na₂SO₄. After concentration, the residue was purified by preparative HPLC to afford (3S,4R)-benzyl 3-hydroxy-4-isobutylpiperidine-1-carboxylate (46.5 mg, 41% yield). LC-MS (3 min) t_{R}=1.80 min., m/z 292 (M+1).

### Examples

### Example 1

### (3R)-1-cyclohexyl-3-(methylamino)propan-2-yl 3-((S)-1-(3-chloro-2-fluorophenyl)-1-hydroxy-5-methoxypentyl)piperidine-1-carboxylate

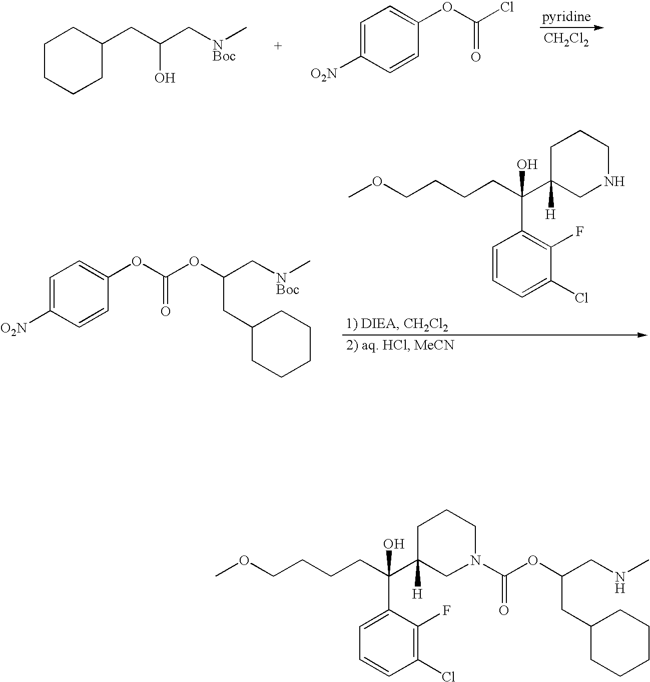

### Step 1. tert-butyl 3-cyclohexyl-2-((4-nitrophenoxy)carbonyloxy)propyl(methyl)carbamate

To a solution of tert-butyl 3-cyclohexyl-2-hydroxypropyl(methyl)carbamate (55 mg, 0.203 mmol) in CH₂Cl₂ (5 mL) was added p-nitrophenylchloroformate (51 mg, 1.25 equiv.) and pyridine (50 μL, excess). The reaction was stirred for 3 h at rt. The mixture was diluted with ether (35 mL), washed with satd aq NaHCO₃ (2×6 mL), water (2×8 mL), and dried over Na₂SO₄. After filtration and concentration, the crude product was purified by chromatography on a 12-g silica gel column eluted with a 0-5% methanol gradient in CH₂Cl₂ to afford product (63 mg, 71%) as a clear oil. LC-MS (3 min) t_{R}=2.43 min, m/z 460.

### Step 2. (3R)-1-(tert-butoxycarbonyl(methyl)amino)-3-cyclohexylpropan-2-yl 3-((S)-1-(3-chloro-2-fluorophenyl)-1-hydroxy-5-methoxypentyl)piperidine-1-carboxylate

A solution of tert-butyl 3-cyclohexyl-2-((4-nitrophenoxy)carbonyloxy)propyl(methyl)carbamate (24 mg, 1 equiv.) in CH₂Cl₂ (3 mL) was added to a solution of (S)-1-(3-chloro-2-fluorophenyl)-5-methoxy-1-((R)-piperidin-3-yl)pentan-1-ol hydrochloride salt (20 mg, 0.055 mmol) in CH₂Cl₂ (3 mL). DIEA (200 μL, excess) was added. The mixture was agitated for 2 h at rt before being concentrated and purified by prep HPLC to afford product (13 mg, 34%). LC-MS (3 min) t_{R}=2.70 min, m/z 627(M+H), 649, 651(M+Na).

### Step 3. (3R)-1-cyclohexyl-3-(methylamino)propan-2-yl 3-((S)-1-(3-chloro-2-fluorophenyl)-1-hydroxy-5-methoxypentyl)piperidine-1-carboxylate

Treatment of (3R)-1-(tert-butoxycarbonyl(methyl)amino)-3-cyclohexylpropan-2-yl 3-((S)-1-(3-chloro-2-fluorophenyl)-1-hydroxy-5-methoxypentyl)piperidine-1-carboxylate with 1:1 MeCN/2N aq HCl as described in Example 7 Step 3 followed by prep HPLC afforded the two isomers of the title compound.

(R)-((S)-1-cyclohexyl-3-(methylamino)propan-2-yl) 3-((S)-1-(3-chloro-2-fluorophenyl)-1-hydroxy-5-methoxypentyl)piperidine-1-carboxylate: LC-MS (3 min) t_{R}=1.62 min, m/z=527, 529. ¹H NMR (CD₃OD) δ=7.50(t, 1H), 7.35(t, 1H), 7.12(t, 1H), 5.14(br t, 1H), 4.46(d, 1H), 4.06(d, 1H), 2.70(s, 3H), 2.17(t, 1H), 1.98(t, 1H).

(R)-((R)-1-cyclohexyl-3-(methylamino)propan-2-yl) 3-((S)-1-(3-chloro-2-fluorophenyl)-1-hydroxy-5-methoxypentyl)piperidine-1-carboxylate: LC-MS (3 min) t_{R}=1.68 min, m/z=527, 529. NMR (CD₃OD) δ=7.51(t, 1H), 7.37(t, 1H), 7.14(t, 1H), 5,11(br d, 1H), 4.44(d, 1H), 4.09(d, 1H), 3.23(s, 2H), 3.18(d, 2H), 2.69(s, 3H).

### Example 2

### (3R)-1-cyclohexyl-3-(methylamino)propan-2-yl 3-((S)-1-(3-chlorophenyl)-1-hydroxy-4-(methoxycarbonylamino)butyl)piperidine-1-carboxylate

The title compound was prepared following procedures analogous to those in Example 1 using methyl (S)-4-(3-chlorophenyl)-4-hydroxy-4-((R)-piperidin-3-yl)butylcarbamate in Step 2 to afford the two isomers of the title compound.

(R)-((S)-1-cyclohexyl-3-(methylamino)propan-2-yl) 3-((S)-1-(3-chlorophenyl)-1-hydroxy-4-(methoxycarbonylamino)butyl)piperidine-1-carboxylate: LC-MS (3 min) t_{R}=1.52 min, m/z=538, 540. ¹H NMR (CD₃OD) δ=7.41(s, 1H), 7.32(t, 1H), 7.25(m, 1H), 5.10(br d, 1H), 4.42(d, 1H), 4.07(d, 1H), 3.60(s, 3H), 3.18(d, 2H), 3.03(t, 2H), 2.71(s, 3H), 2.58(m, 2H), 1.11-0.89(m, 2H).

(R)-((R)-1-cyclohexyl-3-(methylamino)propan-2-yl) 3-((S)-1-(3-chlorophenyl)-1-hydroxy-4-(methoxycarbonylamino)butyl)piperidine-1-carboxylate: LC-MS (3 min) t_{R}=1.55 min, m/z=538, 540. ¹H NMR (CD₃OD) δ=7.42(s, 1H), 7.34-7.20(m, 2H), 5.11(t, 1H), 4.95(d, 1H), 4.04(d, 1H), 3.59(s, 3H), 3.16(t, 2H), 3.03(m, 2H), 2.70(3, 3H), 1.91(m, 2H), 1.09-0.87(m, 2H).

### Example 3

### (2R)-1-cyclohexyl-3-(methylamino)propan-2-yl 2-((R)-1-(3-chloro-2-fluorophenyl)-1-hydroxy-5-methoxypentyl)morpholine-4-carboxylate

The title compound was prepared following procedures analogous to those in Example 1 using (R)-1-(3-chloro-2-fluorophenyl)-5-methoxy-1-((R)-morpholin-2-yl)pentan-1-ol in Step 2 to afford the two isomers of the title compound.

(R)-((S)-1-cyclohexyl-3-(methylamino)propan-2-yl) 2-((R)-1-(3-chloro-2-fluorophenyl)-1-hydroxy-5-methoxypentyl)morpholine-4-carboxylate. LC-MS (3 min) t_{R}=1.58 min, m/z=529, 531. ¹H NMR (CD₃OD) δ=7.53(t, 1H), 7.37(t, 1H), 7.14((t, 1H), 5.14(m, 1H), 4.20(d, 1H), 3.94-3.75(m, 3H), 3.21(s, 3H), 2.71(s, 3H), 2.06(t, 1H), 1.12-0.80(m, 2H).

(R)-((R)-1-cyclohexyl-3-(methylamino)propan-2-yl) 2-((R)-1-(3-chloro-2-fluorophenyl)-1-hydroxy-5-methoxypentyl)morpholine-4-carboxylate. LC-MS (3 min) t_{R}=1.57 min, m/z=529, 531. ¹H NMR (CD₃OD) δ=7.53(t, 1H), 7.37(t, 1H), 7.13(t, 1H), 5.18(m, 1H), 4.22(d, 1H), 3.84(dd, 2H), 3.91(m, 1H), 3.26(s, 3H), 2.72(s, 3H), 2.10(t, 1H), 1.10-0.80(m, 2H).

### Example 4

### (R)-((S)-2-amino-3-cyclohexylpropyl) 3-((S)-1-(3-chloro-2-fluorophenyl)-1-hydroxy-5-methoxypentyl)piperidine-1-carboxylate

The title compound was prepared following procedures analogous to those in Example 1 using (S)-tert-butyl 1-cyclohexyl-3-hydroxypropan-2-ylcarbamate in Step 1 and (S)-1-(3-chloro-2-fluorophenyl)-5-methoxy-1-((R)-piperidin-3-yl)pentan-1-ol in Step 2 to afford the title compound. LC-MS (3 min) t_{R}=1.59 min, m/z=513, 516. ¹H NMR (CD₃OD) δ=7.52(t, 1H), 7.37(t, 1H), 7.14(t, 1H), 7.39(dd, 2H), 4.12(m, 2H), 3.57(s, 1H), 3.24(s, 3H), 2.70(m, 2H), 2.17(tt, 1H), 1.99(t, 1H), 1.91(td, 1H), 0.98(m, 2H), 0.84(m, 1H).

### Example 5

### (R)-((3SR,4RS)-4-isobutylpiperidin-3-yl) 3-((R)-(3-chlorophenyl)(2-(methoxycarbonylamino)ethoxy)methyl)piperidine-1-carboxylate

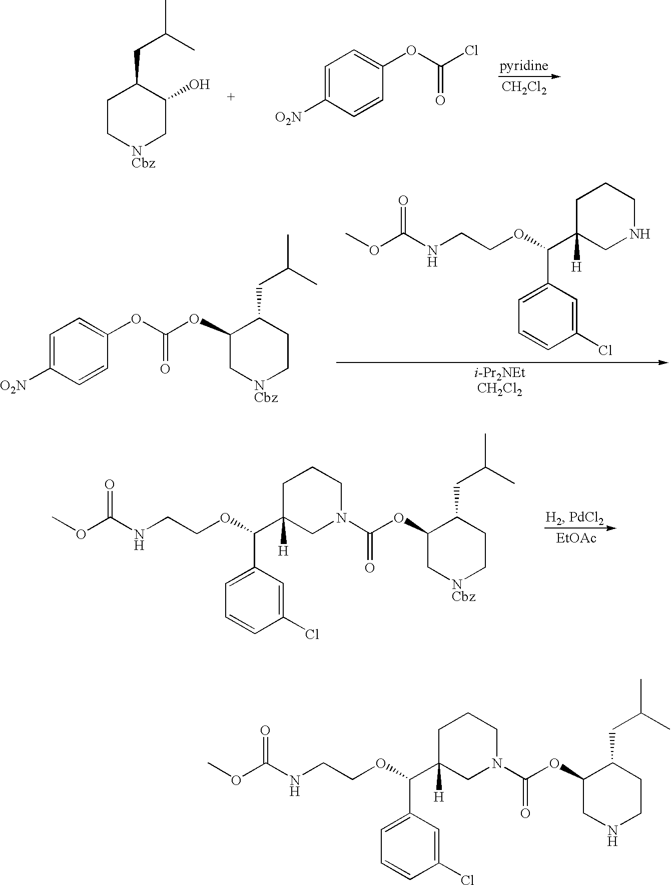

### Step 1. (3S,4R)-benzyl 4-isobutyl-3-((4-nitrophenoxy)carbonyloxy)piperidine-1-carboxylate

To a solution of (3S,4R)-benzyl 3-hydroxy-4-isobutylpiperidine-1-carboxylate (23 mg, 0.079 mmol) in CH₂Cl₂ (3 mL) was added p-nitrophenylchloroformate (20 mg, 1.25 equiv.) and pyridine (25 μL, excess). The reaction was stirred 3 h at r.t. The mixture was diluted by ether (25 mL), washed by sat. NaHCO₃ (2×4 mL), water (2×5 mL), dried over Na₂SO₄. After filtration and concentration, the crude product was purified by Gilson to afford (3S,4R)-benzyl 4-isobutyl-3-((4-nitrophenoxy)carbonyloxy)piperidine-1-carboxylate. LC-MS (3 min) t_{R}=2.25 min., m/z 457 (M+1).

### Step 2. (3S,4R)-benzyl 3-((R)-3-((R)-(3-chlorophenyl)(2-(methoxycarbonylamino)ethoxy)methyl)piperidine-1-carbonyloxy)-4-isobutylpiperidine-1-carboxylate

A solution (3S,4R)-benzyl 4-isobutyl-3-((4-nitrophenoxy)carbonyloxy)piperidine-1-carboxylate from above reaction in CH₂Cl₂ (2 mL) was added to a solution of methyl 2-((R)-(3-chlorophenyl)((R)-piperidin-3-yl)methoxy)ethylcarbamate TFA salt (0.087 mmol) and DIEA (80 μL, excess) in CH₂Cl₂ (3 mL). The mixture was agitated for 2 h at r.t. before being concentrated and purified by Gilson to afford (3S,4R)-benzyl 3-((R)-3-((R)-(3-chlorophenyl)(2-(methoxycarbonylamino)ethoxy)methyl)piperidine-1-carbonyloxy)-4-isobutylpiperidine-1-carboxylate (9 mg, 18% yield for two steps). LC-MS (3 min) t_{R}=2.40 min., m/z 666 (M+Na).

### Step 3. (R)-((3S,4R)-4-isobutylpiperidin-3-yl)-3-((R)-(3-chlorophenyl)(2-(methoxycarbonylamino)ethoxy)methyl)piperidine-1-carboxylate

(3S,4R)-benzyl-3-((R)-3-((R)-(3-chlorophenyl)(2-(methoxycarbonylamino)ethoxy)methyl)piperidine-1-carbonyloxy)-4-isobutylpiperidine-1-carboxylate (9 mg, 0.027 mmol) and palladium(II) chloride (catalytic amount, ˜2 mg) were mixed with EtOAc (3 mL). The flask was evacuated and filled by H₂ gas (3×) and maintained under a H₂ gas atmosphere for 2 h. LC-MS showed most of the starting material had been converted to product. The mixture was concentrated, redissolved in acetonitrile (3 mL), filtered and purified by preparative HPLC to afford (R)-((3S,4R)-4-isobutylpiperidin-3-yl)-3-((R)-(3-chlorophenyl)(2-(methoxycarbonylamino)ethoxy)methyl)piperidine-1-carboxylate (4.8 mg, 67% yield). LC-MS (3 min) t_{R}=1.64 min., m/z 510, 512 (M+1). ¹H NMR (CD₃OD) δ=7.37-7.14(m, 4H), 4.06(d, 1H), 3.63(s, 3H), 3.48(dt, 1H), 3.01(m, 2H), 2.12(m, 1H), 1.90(m, 1H), 0.94(m, 6H).

### Example 6

### (3R)-3-(cyclohexylmethyl)pyrrolidin-3-yl 3(R)-(3-chlorophenyl)(2-(methoxycarbonylamino)ethoxy)methyl)piperidine-1-carboxylate

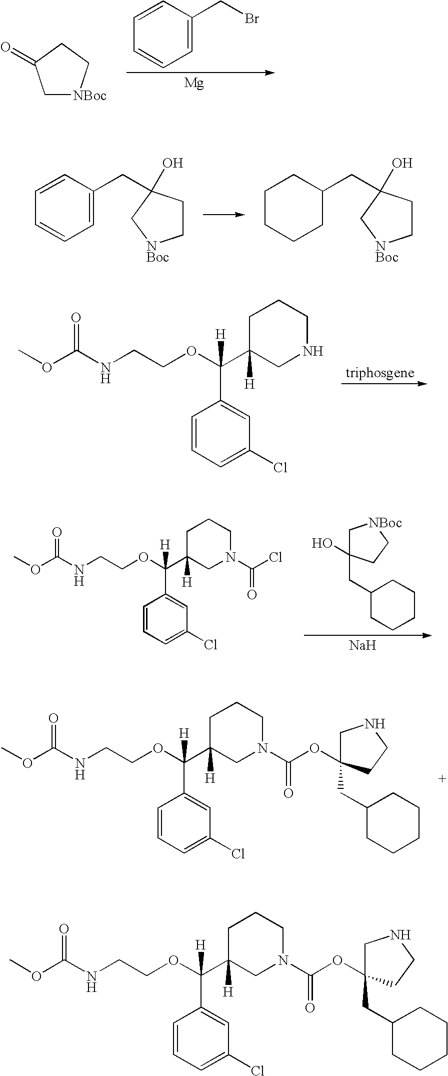

### Step 1. Tert-butyl-3-benzyl-3-hydroxypyrrolidine-1-carboxylate

A crystal of iodine was added to a solution of magnesium shavings (1.28 g, 53.5 mmol) in ether (19 mL), followed by a few mLs of a solution of benzyl bromide (9.24 g, 54 mmol) in ether (38 mL). The reaction was maintained at reflux while the remaining benzyl bromide solution was added dropwise. After addition was complete, the reaction was maintained at reflux with stirring for an additional hour before successively adding ether (38 mL) and, dropwise with vigorous stirring, a solution of tert-butyl 3-oxopyrrolidine-1-carboxylate (10 g, 54 mmol) in ether (38 mL). After addition was complete, the resulting suspension was refluxed for an additional 2 h and stirred for 16 h at room temperature. Then the mixture was filtered. The filter cake was washed well with ether and then hydrolyzed by stirring in a mixture of ice and aq NH₄Cl. The aqueous solution was extracted with ether (3×) and the combined ether extracts were washed with water and brine, dried and concentrated in vacuum. The crude product was purified through column chromatography (4 g, 27%). ¹H NMR (CDCl₃, 400 MHz): δ=1.43 (s, 9H), 1.81 (m, 2H), 2.12 (m, 1H), 2.90 (s, 2H), 3.40 (m, 4H), 7.22 (m, 5H)

### Step 2. Tert-butyl-3-(cyclohexylmethyl)-3-hydroxypyrrolidine-1-carboxylate

Tert-Butyl-3-benzyl-3-hydroxypyrrolidine-1-carboxylate (2 g) was dissolved in AcOH and PtO₂ (150 mg) was added. The mixture was hydrogenated under an initial pressure of 4 MPa at 50° C. The reaction mixture was filtered through a pad of celite and the solvent was removed in vacuum. The crude product was used in the next step. ¹H NMR (CDCl₃, 400 MHz): δ=0.98 (m, 2H), 1.14 (m, 1H), 1.25 (m, 2H), 1.46 (s, 9H), 1.53 (m, 5H), 1.66 (m, 3H), 1.76 (m, 3H), 1.87 (m, 1H), 3.20 (d, 1H), 3.38 (m, 1H), 3.46 (m, 2H)

### Step 3. methyl 2-((R)-((R)-1-(chlorocarbonyl)piperidin-3-yl)(3-chlorophenyl)methoxy)ethylcarbamate

A solution triphosgene (45 mg, 0.153 mmol) in CH₂Cl₂ (5 mL) was added dropwise over 10 min to a solution of methyl 2-((R)-(3-chlorophenyl)((R)-piperidin-3-yl)methoxy)ethylcarbamate (100 mg, 0.307 mmol) and TEA (85.2 μL, 0.614 mmol) in CH₂Cl₂ (3 mL), under nitrogen atmosphere at 0° C. The reaction mixture was stirred at 0° C. to 5° C. for 30 min. The solvent was removed. The residue was dissolved in water and CH₂Cl₂ and the aqueous layer was extracted twice with CH₂Cl₂. The organic layers were combined and washed with saturated brine, and dried over Na₂SO₄. Then, the solvent was removed at 40° C. The crude methyl 2-((R)-((R)-1-(chlorocarbonyl)piperidin-3-yl)(3-chlorophenyl)methoxy)ethylcarbamate (79 mg, 66%) was used in the next step without further purification.

### Step 4. (3R)-3-(cyclohexylmethyl)pyrrolidin-3-yl-3-((R)-(3-chlorophenyl)(2-(methoxycarbonylamino)ethoxy)methyl)piperidine-1-carboxylate

A solution of tert-butyl 3-(cyclohexylmethyl)-3-hydroxypyrrolidine-1-carboxylate (47.88 mg, 0.167 mmol) in toluene was added to a suspension of NaH (60% in mineral oil) in toluene. The mixture was heated to 100° C. for 1 h, then cooled to 0° C. A solution of 2-((R)-((R)-1-(chlorocarbonyl)piperidin-3-yl)(3-chlorophenyl)methoxy)ethylcarbamate (79 mg, 0.203 mmol) in toluene was added over 45 min. The resulting suspension was stirred at room temperature for 19 h. Water was added and the mixture was extracted with EtOAc. The solvent was removed and the isomers of the title compound were separated by preparative HPLC.

(R)-((R)-3-(cyclohexylmethyl)pyrrolidin-3-yl) 3-((R)-(3-chlorophenyl)(2-(methoxycarbonylamino)ethoxy)methyl)piperidine-1-carboxylate (10 mg, 9.2%). LC-MS m/z=536. ¹H NMR (MeOD) δ=1.00 (m, 2H), 1.20 (m, 2H), 1.23-1.40 (m, 5H), 1.55-1.78 (m, 6H), 1.78-1.90 (m, 4H), 2.71 (q, 2H), 3.20-3.28 (m, 2H), 3.30-3.40 (m, 1H), 3.62 (s, 3H), 3.62-3.70 (m, 2H), 3.97-4.07 (m, 2H), 7.21 (d, 1H), 7.26-7.38 (m, 3H).

(R)-((S)-3-(cyclohexylmethyl)pyrrolidin-3-yl) 3-((R)-(3-chlorophenyl)(2-(methoxycarbonylamino)ethoxy)methyl)piperidine-1-carboxylate (3 mg, 2.8%). LC-MS m/z=536. ¹H NMR (MeOD) δ=1.00 (m, 2H), 1.20 (m, 2H), 1.23-1.40 (m, 5H), 1.55-1.78 (m, 6H), 1.78-1.90 (m, 4H), 2.71 (q, 2H), 3.20-3.28 (m, 2H), 3.30-3.40 (m, 1H), 3.62 (s, 3H), 3.62-3.70 (m, 2H), 3.97-4.07 (m, 2H), 7.21 (d, 1H), 7.26-7.38 (m, 3H).

### Example 7

### (R)-((S)-2-amino-3-((R)-tetrahydro-2H-pyran-3-yl)propyl) 3-((R)-(3-chlorophenyl)(2-(methoxycarbonylamino)ethoxy)methyl)piperidine-1-carboxylate

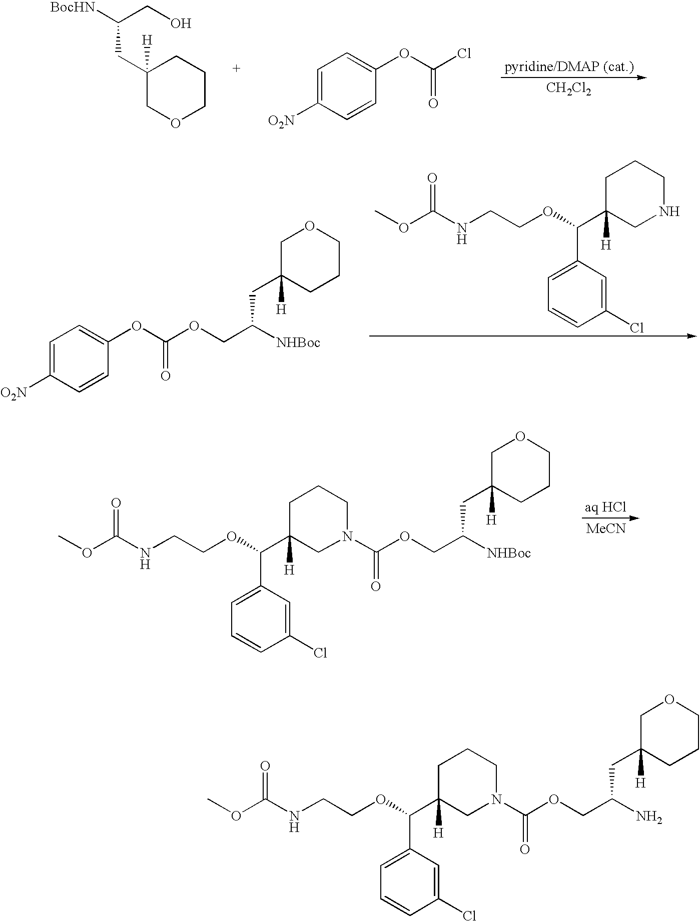

### Step 1. Tert-butyl (S)-1-((4-nitrophenoxy)carbonyloxy)-3-((R)-tetrahydro-2H-pyran-3-yl)propan-2-ylcarbamate

To a solution of tert-butyl (S)-1-hydroxy-3-((R)-tetrahydro-2H-pyran-3-yl)propan-2-ylcarbamate (0.1 mmol) in 1:1 pyridine/CH₂Cl₂ (8 mL) was added p-nitrophenylchloroformate (25 mg, 1.25 equiv.) and DMAP (c.a. 12 mg). After stirring for 3 h at rt, the mixture was diluted with CH₂Cl₂ (15 mL), washed with 5% HCl (2×3 mL), satd aq NaHCO₃ (4 mL), brine (4 mL), and dried over Na₂SO₄. After filtration and concentration, the crude product was purified by prep HPLC to afford product (16.3 mg, 39%). LC-MS (3 min) t_{R}=1.87 min, m/z 448.

### Step 2. (R)-((S)-2-(tert-butoxycarbonylamino)-3-((R)-tetrahydro-2H-pyran-3-yl)propyl) 3-((R)-(3-chlorophenyl)(2-(methoxycarbonylamino)ethoxy)methyl)piperidine-1-carboxylate

A solution of tert-butyl (S)-1-((4-nitrophenoxy)carbonyloxy)-3-((R)-tetrahydro-2H-pyran-3-yl)propan-2-ylcarbamate (16.3 mg, 1 equiv.) in CH₂Cl₂ (3 mL) was added to a solution of methyl 2-((R)-(3-chlorophenyl)((R)-piperidin-3-yl)methoxy)ethylcarbamate TFA salt (16 mg, 0.036 mmol) in CH₂Cl₂ (2 mL), followed by the addition of TEA (100 μL, excess). The mixture was agitated overnight at rt before being concentrated and purified by prep HPLC to afford product (13.1 mg, 59%). LC-MS (3 min) t_{R}=2.05 min, m/z 634, 636(M+Na).

### Step 3. (R)-((S)-2-amino-3-((R)-tetrahydro-2H-pyran-3-yl)propyl) 3-((R)-(3-chlorophenyl)(2-(methoxycarbonylamino)ethoxy)methyl)piperidine-1-carboxylate

(R)-((S)-2-(tert-butoxycarbonylamino)-3-((R)-tetrahydro-2H-pyran-3-yl)propyl) 3-((R)-(3-chlorophenyl)(2-(methoxycarbonylamino)ethoxy)methyl)piperidine-1-carboxylate (13.1 mg, 0.02 mmol) was dissolved in 1:1 acetonitrile/2N aq HCl. The mixture was stirred overnight at rt. The acetonitrile was removed under vacuum and the aqueous residue was basified with 5% aq NaOH solution to pH=8˜9. The mixture was extracted with CH₂Cl₂ (3×6 mL). The combined organic layers were dried over Na₂SO₄. After filtration and concentration, the crude product was purified by prep HPLC to afford product (11 mg, quant). LC-MS (3 min) t_{R}=1.32 min., m/z 512, 514(M+Na).

### Spectral Data on Select Compounds

| Compound Name | Mass Observed | Selected ¹HNMR Resonances |
|---|---|---|
| (R)-((S)-1-cyclohexyl-3- | 527, 529 | 7.50(t, 1H), 7.35(t, 1H), |
| (methylamino)propan-2-yl) 3-((S)-1-(3- | | 7.12(t, 1H), 5.14(br t, 1H), |
| chloro-2-fluorophenyl)-1-hydroxy-5- | | 4.46(d, 1H), 4.06(d, 1H), |
| methoxypentyl)piperidine-1- | | 2.70(s, 3H), 2.17(t, 1H), |
| carboxylate | | 1.98(t, 1H). |
| (R)-((R)-1-cyclohexyl-3- | 527, 529 | 7.51(t, 1H), 7.37(t, 1H), |
| (methylamino)propan-2-yl) 3-((S)-1-(3- | | 7.14(t, 1H), 5.11(br d, 1H), |
| chloro-2-fluorophenyl)-1-hydroxy-5- | | 4.44(d, 1H), 4.09(d, 1H), |
| methoxypentyl)piperidine-1- | | 3.23(s, 2H), 3.18(d, 2H), |
| carboxylate | | 2.69(s, 3H). |
| (R)-((S)-1-cyclohexyl-3- | 538, 540 | 7.41(s, 1H), 7.32(t, 1H), |
| (methylamino)propan-2-yl) 3-((S)-1-(3- | | 7.25(m, 1H), 5.10(br d, 1H), |
| chlorophenyl)-1-hydroxy-4- | | 4.42(d, 1H), 4.07(d, 1H), |
| (methoxycarbonylamino)butyl)piperidine- | | 3.60(s, 3H), 3.18(d, 2H), |
| 1-carboxylate | | 3.03(t, 2H), 2.71(s, 3H), |
| | | 2.58(m, 2H), 1.11-0.89(m, |
| | | 2H). |
| (R)-((R)-1-cyclohexyl-3- | 538, 540 | 7.42(s, 1H), 7.34-7.20(m, |
| (methylamino)propan-2-yl) 3-((S)-1-(3- | | 2H), 5.11(t, 1H), 4.95(d, 1H), |
| chlorophenyl)-1-hydroxy-4- | | 4.04(d, 1H), 3.59(s, 3H), |
| (methoxycarbonylamino)butyl)piperidine- | | 3.16(t, 2H), 3.03(m, 2H), |
| 1-carboxylate | | 2.70(3, 3H), 1.91(m, 2H), |
| | | 1.09-0.87(m, 2H). |
| (R)-((S)-1-cyclohexyl-3- | 529, 531 | 7.53(t, 1H), 7.37(t, 1H), |
| (methylamino)propan-2-yl) 2-((R)-1- | | 7.14((t, 1H), 5.14(m, 1H), |
| (3-chloro-2-fluorophenyl)-1-hydroxy- | | 4.20(d, 1H), 3.94-3.75(m, |
| 5-methoxypentyl)morpholine-4- | | 3H), 3.21(s, 3H), 2.71(s, 3H), |
| carboxylate | | 2.06(t, 1H), 1.12-0.80(m, |
| | | 2H). |
| (R)-((R)-1-cyclohexyl-3- | 529, 531 | 7.53(t, 1H), 7.37(t, 1H), |
| (methylamino)propan-2-yl) 2-((R)-1- | | 7.13(t, 1H), 5.18(m, 1H), |
| (3-chloro-2-fluorophenyl)-1-hydroxy- | | 4.22(d, 1H), 3.84(dd, 2H), |
| 5-methoxypentyl)morpholine-4- | | 3.91(m, 1H), 3.26(s, 3H), |
| carboxylate | | 2.72(s, 3H), 2.10(t, 1H), 1.10- |
| | | 0.80(m, 2H). |
| (R)-((S)-2-amino-3-cyclohexylpropyl) | 513, 516 | 7.52(t, 1H), 7.37(t, 1H), |
| 3-((S)-1-(3-chloro-2-fluorophenyl)-1- | | 7.14(t, 1H), 7.39(dd, 2H), |
| hydroxy-5-methoxypentyl)piperidine- | | 4.12(m, 2H), 3.57(s, 1H), |
| 1-carboxylate | | 3.24(s, 3H), 2.70(m, 2H), |
| | | 2.17(tt, 1H), 1.99(t, 1H), |
| | | 1.91(td, 1H), 0.98(m, 2H), |
| | | 0.84(m, 1H). |
| (R)-((3SR,4RS)-4-isobutylpiperidin-3- | 510, 512 | 7.31(m, 3H), 7.21(m, 1H), |
| yl) 3-((R)-(3-chlorophenyl)(2-(methoxy- | | 4.63(m, 1H), 4.04(d, 1H), |
| carbonylamino)ethoxy)methyl)piperidine- | | 3.95(m, 1H), 3.63(s, 3H), |
| 1-carboxylate | | 3.48(d, 1H), 2.14(m, 1H), |
| | | 1.92(m, 1H), 0.99-0.87(m, |
| | | 6H). |
| (R)-((R)-3-(cyclohexylmethyl)pyrrolidin- | 536 | 1.00 (m, 2H), 1.20 (m, 2H), |
| 3-yl) 3-((R)-(3-chlorophenyl)(2-(methoxy- | | 1.23-1.40 (m, 5H), 1.55-1.78 |
| carbonylamino)ethoxy)methyl)piperidine- | | (m, 6H), 1.78-1.90 (m, 4H), |
| 1-carboxylate | | 2.71 (q, 2H), 3.20-3.28 (m, |
| | | 2H), 3.30-3.40 (m, 1H), 3.62 |
| | | (s, 3H), 3.62-3.70 (m, 2H), |
| | | 3.97-4.07 (m, 2H), 7.21 (d, |
| | | 1H), 7.26-7.38 (m, 3H). |
| (R)-((S)-3-(cyclohexylmethyl)pyrrolidin- | 536 | 1.00 (m, 2H), 1.20 (m, 2H), |
| 3-yl) 3-((R)-(3-chlorophenyl)(2-(methoxy- | | 1.23-1.40 (m, 5H), 1.55-1.78 |
| carbonylamino)ethoxy)methyl)piperidine- | | (m, 6H), 1.78-1.90 (m, 4H), |
| 1-carboxylate | | 2.71 (q, 2H), 3.20-3.28 (m, |
| | | 2H), 3.30-3.40 (m, 1H), 3.62 |
| | | (s, 3H), 3.62-3.70 (m, 2H), |
| | | 3.97-4.07 (m, 2H), 7.21 (d, |
| | | 1H), 7.26-7.38 (m, 3H). |
| (R)-((S)-2-amino-3-((R)-tetrahydro-2H- | 512, 514 | 7.32(m, 3H), 7.22(d, 1H), |
| pyran-3-yl)propyl) 3-((R)-(3- | | 4.37(m, 1H), 3.63(s, 3H), |
| chlorophenyl)(2-(methoxy- | | 3.44(t, 1H), 3.15(t, 1H), |
| carbonylamino)ethoxy)methyl)piperidine- | | 1.96(d, 1H), 1.49(m, 1H), |
| 1-carboxylate | | 1.17(m, 1H). |

### Example 8

### In Vitro Activity Studies—IC₅₀ for Renin

The compounds of the invention have enzyme-inhibiting properties. In particular, they inhibit the action of the natural enzyme renin. The latter passes from the kidneys into the blood where it effects the cleavage of angiotensinogen, releasing the decapeptide angiotensin I, which is then cleaved in the blood, lungs, the kidneys and other organs by angiotensin converting enzyme to form the octapeptide angiotensin II. The octapeptide increases blood pressure both directly by binding to its receptor, causing arterial vasoconstriction, and indirectly by liberating from the adrenal glands the sodium-ion-retaining hormone aldosterone, accompanied by an increase in extracellular fluid volume. That increase can be attributed to the action of angiotensin II. Inhibitors of the enzymatic activity of renin bring about a reduction in the formation of angiotensin I. As a result, a smaller amount of angiotensin II is produced. The reduced concentration of that active peptide hormone is the direct cause of the hypotensive effect of renin inhibitors.

The action of renin inhibitors in vitro is demonstrated experimentally by means of a test that measures the increase in fluorescence of an internally quenched peptide substrate. The sequence of this peptide corresponds to the sequence of human angiotensinogen. The following test protocol is used: All reactions are carried out in a flat bottom white opaque microtiter plate. A 4 μL aliquot of 400 μM renin substrate (DABCYL-γ-Abu-Ile-His-Pro-Phe-His-Leu-Val-Ile-His-Thr-EDANS) in 192 μL assay buffer (50 mM BES, 150 mM NaCl, 0.25 mg/mL bovine serum albumin, pH7.0) is added to 4 μL of test compound in DMSO at various concentrations ranging from 10 μM to 1 nM final concentrations. Next, 100 μL of trypsin-activated recombinant human renin (final enzyme concentration of 0.2-2 nM) in assay buffer is added, and the solution is mixed by pipetting. The increase in fluorescence at 495 nm (excitation at 340 nm) is measured for 60-360 min at rt using a Perkin-Elmer Fusion microplate reader. The slope of a linear portion of the plot of fluorescence increases as a function of time is then determined, and the rate is used for calculating percent inhibition in relation to uninhibited control. The percent inhibition values are plotted as a function of inhibitor concentration, and the IC₅₀ is determined from a fit of this data to a four parameter equation. The IC₅₀ is defined as the concentration of a particular inhibitor that reduces the formation of product by 50% relative to a control sample containing no inhibitor. (Wang G. T. et al. Anal. Biochem. 1993, 210, 351; Nakamura, N. et al. J. Biochem. (Tokyo) 1991, 109, 741; Murakami, K. et al. Anal Biochem. 1981, 110, 232).

### Example 9

### In Vitro Activity Studies—IC₅₀ for Renin

All reactions are carried out in a low volume, black, 384 well microtiter plate (Greiner Bio-one). Compounds were diluted in 100% DMSO, and a 100 nL aliquot of each compound concentration was stamped into the plate using a Hummingbird (Genomic Solutions). 5 μL of 600 pM renin (trypsin-activated recombinant human renin) was then added to the plate, followed by 5 μL of 2 μM substrate (Arg-Glu-Lys(5-FAM)-Ile-His-Pro-Phe-His-Leu-Val-Ile-His-Thr-Lys(5,6-TAMRA)-Arg-CONH₂). Both renin and substrate were made up in buffer containing 50 mM HEPES, 125 mM NaCl, 0.1% CHAPS, with the pH adjusted to 7.4. After 2 hours of reaction at room temperature, the plates were read on a Viewlux™ (PerkinElmer) with an excitation/emission of 485/530 nm, and using a 505 nm cutoff filter. The percent inhibition values are plotted as a function of inhibitor concentration, and the IC₅₀ is determined from a fit of this data to a four parameter equation. The IC₅₀ is defined as the concentration of a particular inhibitor that reduces the formation of product by 50% relative to a control sample containing no inhibitor.

### Example 10

### IC₅₀ Values of the Disclosed Compounds for Renin

The IC₅₀ values of the disclosed compounds for renin were determined according to the protocol described in Example 8 or 9. In these in vitro systems, the compounds of the invention exhibit 50% inhibition at concentrations of from approximately 5000 nM to approximately 0.01 nM. Preferred compounds of the invention exhibit 50% inhibition at concentrations of from approximately 50 nM to approximately 0.01 nM. More preferred compounds of the invention exhibit 50% inhibition at concentrations of from approximately 5 nM to approximately 0.01 nM. Highly preferred compounds of the invention exhibit 50% inhibition at concentrations of from approximately 5 nM to approximately 0.01 nM and exhibit 50% inhibition at concentrations of from approximately 10 nM to approximately 0.01 nM in the in vitro assay in the presence of human plasma described below.

### Example 11

### In Vitro Activity of the Disclosed Compounds in Human Plasma

The action of renin inhibitors in vitro in human plasma is demonstrated experimentally by the decrease in plasma renin activity (PRA) levels observed in the presence of the compounds. Incubations mixtures contain in the final volume of 250 μL 95.5 mM N,N-bis(2-hydroxyethyl)-2-aminoethanesulfonic acid, pH 7.0, 8 mM EDTA, 0.1 mM neomycin sulfate, 1 mg/ml sodium azide, 1 mM phenylmethanesulfonyl fluoride, 2% DMSO and 87.3% of pooled mixed-gender human plasma stabilized with EDTA. For plasma batches with low PRA (less than 1 ng/ml/hr)˜2 pM of recombinant human renin IS added to achieve PRA of 3-4 ng/ml/hr. The cleavage of endogenous angiotensinogen in plasma is carried out at 37° C. for 90 min and the product angiotensin I is measured by competitive radioimmunoassay using DiaSorin PRA kit. Uninhibited incubations containing 2% DMSO and fully inhibited controls with 2 μM of isovaleryl-Phe-Nle-Sta-Ala-Sta-OH are used for deriving percent of inhibition for each concentration of inhibitors and fitting dose-response data into a four parametric model from which IC₅₀ values, defined as concentrations of inhibitors at which 50% inhibition occurs, is determined.

### Example 12

### Efficacy of the Disclosed Inhibitors in a Transgenic Rat Model

The efficacy of the renin inhibitors may also be evaluated in vivo in double transgenic rats engineered to express human renin and human angiotensinogen (Bohlender J, Fukamizu A, Lippoldt A, Nomura T, Dietz R, Menard J, Murakami K, Luft F C, Ganten D. High human renin hypertension in transgenic rats. Hypertension 1997, 29, 428-434).

Experiments could be conducted in 5-10 week-old double transgenic rats (dTGRs). The model has been described in detail earlier. Briefly, the human renin construct that may be used to generate transgenic animals (hRen) is made up of the entire genomic human renin gene (10 exons and 9 introns), with 3.0 kB of the 5′-promoter region and 1.2 kB of 3′ additional sequences. A human angiotensinogen construct containing the entire human angiotensinogen gene (5 exons and 4 introns), with 1.3 kB of 5′-flanking and 2.4 kB of 3′-flanking sequences may be used to generate rats producing human angiotensinogen (hAogen). The hRen and hAogen rats may be rederived using embryo transfer from breeding pairs obtained under license from Ascencion Gmbh (Germany). The hAogen and hRen may then be crossed to produce the double transgenic dTGR) off-spring. The dTGr rats should be maintained on irradiated rodent chow (5VO2, Purina Mills Inc) and normal water. Radio telemetry transmitters (TA11PAC40, Data Sciences International) may be surgically implanted at 5-6 weeks of age. The telemetry system can provide 24-h recordings of systolic, mean, diastolic arterial pressure (SAP, MAP, DAP, respectively) and heart rate (HR). Prior to dosing, baseline hemodynamic measures should be obtained for 24 hours. Rats may then be dosed orally with vehicle or drug and monitored up to 48 hours post-dose.

### Example 13

### In Vivo Activity

The cardiac and systemic hemodynamic efficacy of selective renin inhibitors can be evaluated in vivo in sodium-depleted, normotensive cynomolgus monkeys and in sodium-depleted, normotensive beagle dogs following a single oral and intravenous administration of the test compound. Arterial blood pressure can be monitored by telemetry in freely moving, conscious animals.

Cynomolgus Monkey: Six male naïve cynomolgus monkeys weighing between 2.5 and 3.5 kg can be used in the studies. At least 4 weeks before the experiment, the monkeys are anesthetized with ketamine hydrochloride (15 mg/kg, i.m.) and xylazine hydrochloride (0.7 mg/kg, i.m.), and are implanted into the abdominal cavity with a transmitter (Model #TL11M2-D70-PCT, Data Sciences, St. Paul, Minn.). A pressure catheter would be inserted into the lower abdominal aorta via the femoral artery. The bipotential leads would be placed in Lead II configuration. The animals would be housed under constant temperature (19-25° C.), humidity (>40%) and lighting conditions (12 h light and dark cycle), and fed once daily with free access to water. The animals would be sodium depleted by placing them on a low sodium diet (0.026%, Expanded Primate Diet 829552 MP-VENaCl (P), Special Diet Services, Ltd., UK) 7 days before the experiment. Furosemide (3 mg/kg, intramuscularly i.m., Aventis Pharmaceuticals) would be administered at −40 h and −16 h prior to administration of test compound.

For oral dosing, the renin inhibitors can be formulated in 0.5% methylcellulose at dose levels of 10 and 30 mg/kg (5 mL/kg) by infant feeding tubes. For intravenous delivery, a silastic catheter can be implanted into posterior vena cava via a femoral vein. Said catheter would be attached to the delivery pump via a tether system and a swivel joint. Test compound (dose levels of 0.1 to 10 mg/kg, formulated at 5% dextrose) can be administered by continuous infusion (1.67 mL/kg/h) or by bolus injection (3.33 mL/kg in 2 min).

Arterial blood pressures (systolic, diastolic and mean) and body temperature can be recorded continuously at 500 Hz and 50 Hz, respectively, using the Dataquest™ A.R.T. (Advanced Research Technology) software. Heart rate is derived from the phasic blood pressure tracing. During the recording period, the monkeys can be kept in a separate room without human presence to avoid pressure changes secondary to stress. All data would be expressed as mean±SEM. Effects of the renin inhibitors on blood pressure can be assessed by ANOVA, taking into account the factors dose and time compared with the vehicle group.

Beagle Dogs: Non-naive Beagle dogs (2 per sex) weighing between 9 and 11 kg can be used in the studies. Each animal would be implanted subcutaneously with a telemetry transmitter (Data Sciences) and a blood pressure catheter would be inserted into the left femoral artery. The electrocardiogram leads would also be tunneled subcutaneously to the appropriate anatomical regions. The animals can be housed under constant temperature and lighting conditions, fed once daily, and allowed free access to water. A sodium depleted state can be produced by placing them on a low-sodium diet (<4 meq/day, a combination of canned Prescription Diet canine h/d, from Hill's Pet Products and dry pellets from Bio-Serv Inc., Frenchtown, N.J.) beginning 10 days before the experiment. Furosemide (3 mg/kg i.m.; Aventis Pharmaceuticals) can be administered at −40 h and −16 h prior to administration of test compound.

A renin inhibitor can be orally administered by orogastric gavage to all overnight fasted animals at a dose level of 30 mg/kg (4 mL/kg formulated in 0.5% methylcellulose). Food can be given 4 h postdose. In some experiments, the renin inhibitor can be administered by bolus i. v. at increasing dose levels of 1, 3 and 6 mg/kg (2, 6 and 20 mg/mL formulated in sterile saline). Cardiovascular parameters would be collected continuously for at least 80 min predose and 3 h postdose, followed by every 10 min for 5 h and every 30 min for 16 h postdose. The Dataquest™ ART (version 2.2) software package from DSI (Data Sciences International) can be used to collect telemetered cardiovascular data.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A compound according to the formula: 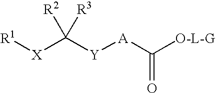
or an enantiomer, diastereomer, or pharmaceutically acceptable salt thereof;
wherein:
R¹ is:
a) (C₁-C₁₂)alkyl, (C₃-C₇)cycloalkyl, (C₄-C₁₂)cycloalkylalkyl, halo(C₁-C₁₂)alkyl, halo(C₃-C₇)cycloalkyl, halo(C₄-C₁₂)cycloalkylalkyl or saturated heterocyclyl, each optionally substituted with 1 to 5 groups independently selected from the group consisting of: halogen, (C₁-C₆)alkyl, halo(C₁-C₆)alkyl and oxo; or
b) phenyl, napthyl, heteroaryl or bicyclic heteroaryl, each substituted with n groups represented by R¹¹, wherein n is an integer from 0 to 5, and wherein each R¹¹ is independently selected from the groups consisting of:
1) fluoride, chloride, bromide, iodide, cyano, nitro, amino, hydroxy, carboxy, (C₁-C₈)alkyl, (C₃-C₈)cycloalkyl, (C₁-C₃)alkyl(C₃-C₈)cycloalkyl, di(C₁-C₃)alkyl(C₃-C₈)cycloalkyl, (C₄-C₈)cycloalkylalkyl, (C₂-C₆)alkenyl, (C₅-C₈)cycloalkenyl, (C₅-C₈)cycloalkylalkenyl, (C₂-C₈)alkynyl, (C₃-C₈)cycloalkyl(C₂-C₄)alkynyl, halo(C₁-C₈)alkyl, halo(C₃-C₆)cycloalkyl, halo(C₄-C₈)cycloalkylalkyl, (C₁-C₃)alkyl(C₄-C₈)cycloalkylalkyl, di(C₁-C₃)alkyl(C₄-C₈)cycloalkylalkyl, halo(C₂-C₈)alkenyl, halo(C₅-C₈)cycloalkenyl, halo(C₆-C₈)cycloalkenylalkyl, halo(C₃-C₈)alkynyl, halo(C₅-C₈)cycloalkylalkynyl, (C₁-C₈)alkoxy, (C₃-C₈)cycloalkoxy, (C₄-C₈)cycloalkylalkoxy, (C₁-C₃)alkyl(C₃-C₈)cycloalkoxy, (C₁-C₃)alkyl(C₄-C₈)cycloalkylalkoxy, di(C₁-C₃)alkyl(C₃-C₈)-cycloalkoxy, di(C₁-C₃)alkyl(C₄-C₈)cycloalkylalkoxy, halo(C₁-C₆)alkoxy, halo(C₃-C₆)cycloalkoxy, halo(C₄-C₈)cycloalkylalkoxy, (C₁-C₈)alkylthio, (C₃-C₈)cycloalkylthio, (C₄-C₈)cycloalkylalkylthio, (C₁-C₃)alkyl(C₃-C₈)cycloalkylthio, (C₁-C₃)alkyl(C₄-C₈)cycloalkylalkylthio, di(C₁-C₃)alkyl(C₃-C₈)cycloalkylthio, di(C₁-C₃)alkyl(C₄-C₈)cycloalkylalkylthio, halo(C₁-C₈)alkylthio, halo(C₃-C₈)cycloalkylthio, halo(C₄-C₈)cycloalkylalkylthio, (C₁-C₈)alkanesulfinyl, (C₃-C₈)cycloalkanesulfinyl, (C₄-C₈)cycloalkylalkanesulfinyl, (C₁-C₃)alkyl(C₃-C₈)cycloalkanesulfinyl, (C₁-C₃)alkyl(C₄-C₈)cycloalkylalkanesulfinyl, di(C₁-C₃)alkyl(C₃-C₈)cycloalkanesulfinyl, di(C₁-C₃)alkyl(C₄-C₈)cycloalkylalkanesulfinyl, halo(C₁-C₈)alkanesulfinyl, halo(C₃-C₈)cycloalkanesulfinyl, halo(C₄-C₈)cycloalkylalkanesulfinyl, (C₁-C₈)alkanesulfonyl, (C₃-C₈)cycloalkanesulfonyl, (C₄-C₈)cycloalkylalkanesulfonyl, (C₁-C₃)alkyl(C₃-C₈)cycloalkanesulfonyl, (C₁-C₃)alkyl(C₄-C₈)cycloalkylalkanesulfonyl, di(C₁-C₃)alkyl(C₃-C₈)cycloalkanesulfonyl, di(C₁-C₃)alkyl(C₄-C₈)cycloalkylalkanesulfonyl, halo(C₁-C₈)alkanesulfonyl, halo(C₃-C₈)cycloalkanesulfonyl, halo(C₄-C₈)cycloalkylalkanesulfonyl, (C₁-C₈)alkylamino, di(C₁-C₈)alkylamino, (C₁-C₆)alkoxy(C₁-C₆)alkoxy, halo(C₁-C₆)alkoxy(C₁-C₆)alkoxy, (C₁-C₈)alkoxycarbonyl, aminocarbonyl, (C₁-C₈)alkylaminocarbonyl, di(C₁-C₈)alkylaminocarbonyl, piperidino, pyrrolidino, cyano(C₁-C₆)alkyl, hydroxy(C₁-C₆)alkyl, carboxy(C₁-C₆)alkyl, (C₁-C₈)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkylthio(C₁-C₆)alkyl, halo(C₄-C₈)cycloalkylalkylthio(C₁-C₆)alkyl, (C₁-C₈)alkanesulfinyl(C₁-C₆)alkyl, (C₃-C₈)cycloalkanesulfinyl(C₁-C₆)alkyl, (C₄-C₈)cycloalkylalkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl (C₁-C₆)alkyl, (C₁-C₈)alkanesulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkanesulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl, (C₁-C₈)acylamino(C₁-C₆)alkyl, piperidino(C₁-C₆)alkyl, pyrrolidino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, or di(C₁-C₈)alkylaminocarboxy(C₁-C₆)alkyl; and
2) phenyl, napthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenyl sulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, bicyclic heteroaryl(C₁-C₃)alkyl, phenyl(C₁-C₃)alkoxy, naphthyl(C₁-C₃)alkoxy, heteroaryl(C₁-C₃)alkoxy, and bicyclic heteroaryl(C₁-C₃)alkoxy, each optionally substituted with 1 to 5 groups independently selected from the group consisting of: fluoride, chloride, cyano, (C₁-C₆)alkyl, halo(C₁-C₆)alkyl, (C₁-C₆)alkoxy, halo(C₁-C₆)alkoxy, (C₁-C₆)alkanesulfonyl, (C₁-C₆)alkoxycarbonyl and aminocarbonyl; with the proviso that when R¹ is imidazole, then R³ is not —H or (C₁-C₆)alkyl; X and Y are each independently —CH₂— or a single bond;
R² is a substituted or unsubstituted (C₁-C₁₂)alkyl, (C₂-C₁₂)alkenyl, (C₂-C₁₂)alkynyl, (C₁-C₁₂)alkoxy, (C₂-C₁₂)alkenyloxy, (C₁-C₁₂)alkylthio, (C₁-C₆)alkoxy(C₁-C₆)alkyl, (C₁-C₆)alkylthio(C₁-C₆)alkyl, (C₁-C₆)alkoxy(C₁-C₆)alkoxy, (C₁-C₆)alkoxy(C₁-C₆)alkylthio, (C₁-C₆)alkylthio(C₁-C₆)alkoxy, (C₁-C₆)alkylthio(C₁-C₆)alkylthio, (C₁-C₄)alkoxy(C₁-C₄)alkoxy(C₁-C₄)alkyl, aminocarbonylamino(C₁-C₁₂)alkyl, aminocarbonylamino(C₁-C₁₂)alkoxy, aminocarbonylamino(C₁-C₁₂)alkylthio, (C₁-C₆)alkanoylamino(C₁-C₆)alkyl, (C₁-C₆)alkanoylamino(C₁-C₆)alkoxy, (C₁-C₆)alkanoylamino(C₁-C₆)alkylthio, (C₃-C₄)cycloalkylcarbonylamino(C₁-C₆)alkyl, (C₃-C₄)cycloalkylcarbonylamino(C₁-C₆)alkoxy, (C₃-C₄)cycloalkylcarbonylamino(C₁-C₆)alkylthio, aminosulfonylamino(C₁-C₁₂)alkyl, aminosulfonylamino(C₁-C₁₂)alkoxy, aminosulfonylamino(C₁-C₁₂)alkylthio, (C₁-C₆)alkanesulfonylamino(C₁-C₆)alkyl, (C₁-C₆)alkane-sulfonylamino(C₁-C₆)-alkoxy, (C₁-C₆)alkanesulfonylamino(C₁-C₆)alkylthio, formylamino(C₁-C₆)alkyl, formylamino(C₁-C₆)alkoxy, formylamino(C₁-C₆)alkylthio, (C₁-C₆)alkoxycarbonylamino(C₁-C₆)alkyl, (C₁-C₆)alkoxycarbonylamino(C₁-C₆)alkoxy, (C₁-C₆)alkoxycarbonylamino(C₁-C₆)alkylthio, (C₁-C₆)alkylaminocarbonylamino(C₁-C₆)alkyl, di(C₁-C₆)alkylaminocarbonylamino(C₁-C₆)alkyl, (C₁-C₆)alkylaminocarbonylamino(C₁-C₆)alkoxy, di(C₁-C₆)alkylaminocarbonylamino(C₁-C₆)alkoxy, (C₁-C₆)alkylaminocarbonylamino(C₁-C₆)alkylthio, di(C₁-C₆)alkylaminocarbonylamino(C₁-C₆)alkylthio, aminocarbonyl(C₁-C₆)alkyl, aminocarbonyl(C₁-C₆)alkoxy, aminocarbonyl(C₁-C₆)alkylthio, (C₁-C₆)alkylaminocarbonyl(C₁-C₆)alkyl, (C₁-C₆)alkylaminocarbonyl(C₁-C₆)alkoxy, (C₁-C₆)alkylaminocarbonyl(C₁-C₆)alkylthio, aminocarboxy(C₁-C₆)alkylaminocarboxy(C₁-C₆)alkoxy, aminocarboxy(C₁-C₆)alkylthio, (C₁-C₆)alkylaminocarboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₁₂)alkoxycarbonylamino, (C₁-C₁₂)alkylaminocarbonylamino, (C₁-C₈)oxoalkyl, or (C₁-C₁₂)alkanoylamino; wherein each group represented by R² is substituted by 0 to 6 groups selected from: halogen, cyano, hydroxyl, (C₁-C₃)alkoxy, (C₃-C₆)cycloalkyl, (C₃-C₆)cycloalkoxy, halo(C₁-C₃)alkoxy, halo(C₃-C₆)cycloalkyl or halo(C₃-C₆)cycloalkoxy; wherein any thio-moiety of said unsubstituted or substituted R² group is optionally and independently replaced by —S(O)— or —S(O)₂—; and wherein any carbonyl moiety of said unsubstituted or substituted R² group is optionally and independently replaced by a thiocarbonyl moiety;
R³ is:
a) —H, halogen, (C₁-C₆)alkyl, (C₁-C₆)alkoxy, hydroxyl, hydroxy(C₁-C₆)alkyl, hydroxy(C₁-C₆)alkoxy, (C₁-C₆)alkanoylamino, (C₁-C₆)alkoxycarbonylamino, (C₁-C₆)alkylaminocarbonylamino, di(C₁-C₆)alkylaminocarbonylamino, (C₁-C₆)alkanesulfonylamino, (C₁-C₆)alkylaminosulfonylamino, or di(C₁-C₆)alkylaminosulfonylamino; or
b) phenylamino or heteroarylamino in which each phenylamino or heteroarylamino group is optionally substituted with 1 to 5 groups independently selected from the group consisting of: fluoride, chloride, bromide, iodide, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, and di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkylalkylthio-(C₁-C₆)alkyl, halo(C₁-C₈)alkylthio(C₁-C₆)alkyl, halo(C₃-C₈)cycloalkylthio(C₁-C₆)alkyl, halo(C₄-C₈)cycloalkylalkylthio(C₁-C₆)alkyl, (C₁-C₈)alkanesulfinyl(C₁-C₆)alkyl, (C₃-C₈)cycloalkanesulfinyl(C₁-C₆)alkyl, (C₄-C₈)cycloalkylalkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkanesulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkanesulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl(C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, and di(C₁-C₈)alkylaminocarboxy(C₁-C₆)alkyl;
provided that:
1) when R³ is hydroxyl, halogen or optionally substituted phenylamino or heteroarylamino, then R² is not a substituted or unsubstituted (C₁-C₁₂)alkoxy, (C₁-C₆)alkoxy(C₁-C₆)alkoxy, (C₁-C₆)alkylthio(C₁-C₆)alkoxy, aminocarbonylamino(C₁-C₁₂)alkyl, aminocarbonylamino(C₁-C₁₂)alkoxy, (C₁-C₆)alkanoylamino(C₁-C₆)alkoxy, (C₃-C₄)cycloalkylcarbonylamino(C₁-C₆)alkoxy, aminosulfonylamino(C₁-C₁₂)alkoxy, (C₁-C₆)alkanesulfonylamino(C₁-C₆)alkoxy, formylamino(C₁-C₆)alkoxy, (C₁-C₆)alkoxycarbonylamino(C₁-C₆)alkoxy, (C₁-C₆)alkylaminocarbonylamino(C₁-C₆)alkoxy, di(C₁-C₆)alkylaminocarbonylamino(C₁-C₆)alkoxy, aminocarbonyl(C₁-C₆)alkoxy, (C₁-C₆)alkylaminocarbonyl(C₁-C₆)alkoxy, aminocarboxy(C₁-C₆)alkoxy, (C₁-C₆)alkylaminocarboxy(C₁-C₆)alkoxy, (C₁-C₁₂)alkoxycarbonylamino, (C₁-C₁₂)alkylaminocarbonylamino, or (C₁-C₁₂)alkanoylamino;
2) when R³ is hydroxyl, halogen, or optionally substituted phenylamino or heteroarylamino, then R² is not a unsubstituted or substituted (C₁-C₁₂)alkylthio, (C₁-C₆)alkoxy(C₁-C₆)alkylthio, (C₁-C₆)alkylthio(C₁-C₆)alkylthio, aminocarbonylamino(C₁-C₁₂)alkylthio, (C₁-C₆)alkanoylamino(C₁-C₆)alkylthio, (C₃-C₄)cycloalkylcarbonylamino(C₁-C₆)alkylthio, aminosulfonylamino(C₁-C₁₂)alkylthio, (C₁-C₆)alkanesulfonylamino(C₁-C₆)alkylthio, formylamino(C₁-C₆)alkylthio, (C₁-C₆)alkoxycarbonylamino(C₁-C₆)alkylthio, (C₁-C₆)alkylaminocarbonylamino(C₁-C₆)alkylthio, di(C₁-C₆)alkylaminocarbonylamino(C₁-C₆)alkylthio, aminocarbonyl(C₁-C₆)alkylthio, (C₁-C₆)alkylaminocarbonyl(C₁-C₆)alkylthio, aminocarboxy(C₁-C₆)alkylthio or (C₁-C₆)alkylaminocarboxy(C₁-C₆)alkylthio, wherein the thin-moiety is replaced by —S(O)— or —S(O)₂—; and
3) when R³ is hydroxyl, halogen, or optionally substituted phenylamino or heteroarylamino, then R² is not a unsubstituted or substituted aminocarbonylamino(C₁-C₁₂)alkoxy, aminocarbonylamino(C₁-C₁₂)alkylthio, (C₁-C₆)alkanoylamino(C₁-C₆)alkoxy, (C₁-C₆)alkanoylamino(C₁-C₆)alkylthio, (C₃-C₄)cycloalkylcarbonylamino(C₁-C₆)alkoxy, (C₃-C₄)cycloalkylcarbonylamino(C₁-C₆)alkylthio, formylamino(C₁-C₆)alkoxy, formylamino(C₁-C₆)alkylthio, (C₁-C₆)alkoxycarbonylamino(C₁-C₆)alkoxy, (C₁-C₆)alkoxycarbonylamino(C₁-C₆)alkylthio, (C₁-C₆)alkylaminocarbonylamino(C₁-C₆)alkoxy, di(C₁-C₆)alkylaminocarbonylamino(C₁-C₆)alkoxy, (C₁-C₆)alkylaminocarbonylamino(C₁-C₆)alkylthio, di(C₁-C₆)alkylaminocarbonylamino(C₁-C₆)alkylthio, aminocarbonyl(C₁-C₆)alkoxy, aminocarbonyl(C₁-C₆)alkylthio, (C₁-C₆)alkylaminocarbonyl-(C₁-C₆)alkoxy, (C₁-C₆)alkylaminocarbonyl(C₁-C₆)alkylthio, aminocarboxy(C₁-C₆)alkoxy, aminocarboxy(C₁-C₆)alkylthio, (C₁-C₆)alkylaminocarboxy(C₁-C₆)alkoxy, (C₁-C₆)alkylaminocarboxy(C₁-C₆)alkylthio, (C₁-C₁₂)alkoxycarbonylamino, (C₁-C₁₂)alkylaminocarbonylamino, or (C₁-C₁₂)alkanoylamino, wherein the carbonyl moiety is replaced by a thiocarbonyl moiety;
A is a saturated or unsaturated 4-, 5-, 6-, or 7-membered ring which is optionally bridged by (CH₂)ₘ via bonds to two members of said ring, wherein said ring is composed of carbon atoms, and 0-2 hetero atoms selected from 0, 1, or 2 nitrogen atoms, 0 or 1 oxygen atoms, and 0 or 1 sulfur atoms, said ring being optionally substituted with up to four moieties independently selected from the group consisting of: halogen, (C₁-C₆)alkyl, halo(C₁-C₆)alkyl, and oxo;
m is 1 to 3;
the carbonyl carbon and Y are attached to carbon or nitrogen atoms in ring A in a 1,2-, 1,3- or 1,4-relationship;
L is: 1) a linear (C₂-C₄)alkyl chain when G is —OH, —OR⁹, —NH₂, —NHR⁹, —NR⁹R¹⁰, —NHC(═NH)NH₂, or —NHC(═NH)NHR⁹; or 2) a linear (C₁-C₃)alkyl chain when G is —C(═NH)NH₂ or —C(═NH)NHR⁹; or
one or more of the carbon atoms of L may be part of a 3-, 4-, 5-, 6-, or 7-membered saturated ring composed of carbon atoms, and 0-2 hetero atoms selected from 0 or 1 nitrogen atoms, 0 or 1 oxygen atoms, and 0 or 1 sulfur atoms;
L is substituted by 1-4 groups independently selected from R⁵, R⁶, R⁷, and R⁸;
each R⁵, R⁶, R⁷, and R⁸ is independently selected from:
1) hydrogen;
2) (C₁-C₁₂)alkyl, (C₃-C₈)cycloalkyl, (C₃-C₈)cycloalkyl(C₁-C₃)alkyl, (C₂-C₁₂)alkenyl, (C₅-C₈)cycloalkyl(C₁-C₃)alkenyl, (C₂-C₁₂)alkynyl, (C₃-C₈)cycloalkyl(C₁-C₃)alkynyl, (C₄-C₁₂)bicycloalkyl(C₁-C₃)alkyl, (C₈-C₁₄)tricycloalkyl(C₁-C₃)alkyl, (C₁-C₆)alkoxy(C₁-C₆)alkyl, (C₃-C₈)cycloalkoxy(C₁-C₃)alkyl, (C₁-C₆)alkylthio(C₁-C₆)alkyl, (C₃-C₈)cycloalkylthio(C₁-C₃)alkyl, saturated heterocyclyl, and saturated heterocyclyl(C₁-C₃)alkyl;
wherein each R⁵, R⁶, R⁷ and R⁸ is optionally and independently substituted by a group selected from: halogen, cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, halo(C₃-C₆)cycloalkoxy; and divalent sulfur atoms are optionally oxidized to sulfoxide or sulfone; and
3) phenyl, naphthyl, heteroaryl, phenyl(C₁-C₃)alkyl, naphthyl(C₁-C₃)alkyl, and heteroaryl(C₁-C₃)alkyl; each optionally substituted with 1 to 3 groups independently selected from:
fluoride, chloride, bromide, iodide, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfanyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkythio(C₁-C₆)alkyl, halo(C₄-C₈)-cycloalkylalkylthio(C₁-C₆)alkyl, (C₁-C₈)alkanesulfinyl(C₁-C₆)alkyl, (C₃-C₈)cycloalkanesulfinyl(C₁-C₆)alkyl, (C₄-C₈)cycloalkylalkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkanesulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkanesulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl(C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, di(C₁-C₈)alkylaminocarboxy(C₁-C₆)alkyl, phenyl, napthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicycle heteroarylsulfonyl, phenyl(C₁-C₃)alkyl, napthyl(C₁-C₃)alkyl, heteroaryl(C₁-C₃)alkyl, and bicyclic heteroaryl(C₁-C₃)alkyl; wherein the aromatic and heteroaromatic groups are optionally substituted with 1 to 3 groups independently selected from: fluoride, chloride, cyano, (C₁-C₃)alkyl, halo(C₁-C₃)alkyl, (C₁-C₃)alkoxy, halo(C₁-C₃)-alkoxy, (C₁-C₃)alkanesulfonyl, and (C₁-C₃)alkoxycarbonyl;
G is —OH, —OR⁹, —NH₂, —NHR⁹, —NR⁹R¹⁰, —C(═NH)NH₂, —C(═NH)NHR⁹, —NHC(═NH)NH₂, or —NHC(═NH)NHR⁹;
R⁹ is:
a) (C₁-C₁₂)alkyl, (C₄-C₁₂)cycloalkylalkyl, halo(C₁-C₁₂)alkyl, halo(C₄-C₁₂)cycloalkylalkyl, (C₂-C₁₂)alkenyl, (C₅-C₁₂)cycloalkylalkenyl, halo(C₂-C₁₂)alkenyl, halo(C₅-C₁₂)cycloalkylalkenyl, (C₂-C₁₂)alkynyl, (C₅-C₁₂)cycloalkylalkynyl, halo(C₂-C₁₂)alkynyl, halo(C₅-C₁₂)cycloalkylalkynyl, (C₁-C₆)alkoxy(C₁-C₆)alkyl, halo(C₁-C₆)alkoxy(C₁-C₆)alkyl, (C₁-C₆)alkylthio(C₁-C₆)alkyl, halo(C₁-C₆)alkylthio(C₁-C₆)alkyl, (C₁-C₆)alkanesulfinyl(C₁-C₆)alkyl, halo(C₁-C₆)alkanesulfinyl(C₁-C₆)alkyl, (C₁-C₆)alkanesulfonyl(C₁-C₆)alkyl, halo(C₁-C₆)alkanesulfonyl(C₁-C₆)alkyl, aminocarbonyl(C₁-C₆)alkyl, (C₁-C₆)alkylaminocarbonyl(C₁-C₆)alkyl, di(C₁-C₆)alkylaminocarbonyl(C₁-C₆)alkyl, cyano(C₁-C₆)alkyl, carboxy(C₁-C₆)alkyl, (C₁-C₆)alkoxycarbonyl(C₁-C₆)alkyl, saturated heterocyclyl, or saturated heterocyclyl(C₁-C₆)alkyl; or
b) phenyl, naphthyl, heteroaryl, phenyl(C₁-C₃)alkyl, naphthyl(C₁-C₃)alkyl, or heteroaryl(C₁-C₃)alkyl, each optionally and independently substituted by 1 to 3 groups selected from:
1) fluoride, chloride, bromide, iodide, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkylthio(C₁-C₆)alkyl, halo(C₄-C₈)cycloalkylalkylthio(C₁-C₆)alkyl, (C₁-C₈)alkanesulfinyl(C₁-C₆)alkyl, (C₃-C₈)cycloalkanesulfinyl(C₁-C₆)alkyl, (C₄-C₈)cycloalkylalkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkanesulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkanesulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl, (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl and di(C₁-C₈)alkylaminocarboxy(C₁-C₆)alkyl; and
2) phenyl, napthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, napthyl(C₁-C₃)alkyl, heteroaryl(C₁-C₃)alkyl, and bicyclic heteroaryl(C₁-C₃)alkyl, each optionally substituted with 1 to 3 groups independently selected from: fluoride, chloride, cyano, (C₁-C₃)alkyl, halo(C₁-C₃)alkyl, (C₁-C₃)alkoxy, halo(C₁-C₃)alkoxy, (C₁-C₃)alkanesulfonyl, and (C₁-C₃)-alkoxycarbonyl; or
c) R⁹ taken together with one of R⁵, R⁶, R⁷ or R⁸ and their intervening atoms form a saturated 3-, 4-, 5-, 6-, or 7-membered “L-G ring” comprising 3 to 7 carbon atoms, and 1 or 2 heteroatoms selected from 0 or 1 nitrogen atoms, 0 or 1 oxygen atoms, and 0 or 1 sulfur atoms; said L-G ring being optionally substituted with 1 to 4 groups selected from: halogen, (C₁-C₈)alkyl, halo(C₁-C₈)alkyl, (C₃-C₈)cycloalkyl, (C₁-C₂)alkyl(C₃-C₆)cycloalkyl, halo(C₃-C₈)cycloalkyl, hydroxy(C₃-C₈)cycloalkyl, (C₃-C₈)cycloalkyl(C₁-C₃)alkyl, halo(C₃-C₈)cycloalkyl(C₁-C₃)alkyl, hydroxy(C₃-C₈)cycloalkyl(C₁-C₃)alkyl, (C₁-C₈)alkoxy, halo(C₁-C₈)alkoxy, (C₃-C₈)cycloalkoxy, halo(C₃-C₈)cycloalkoxy, hydroxy(C₃-C₈)cycloalkoxy, (C₁-C₈)alkoxy(C₁-C₃)alkyl, halo(C₁-C₈)alkoxy(C₁-C₃)alkyl, (C₃-C₈)cycloalkoxy(C₁-C₃)alkyl, halo(C₃-C₈)cycloalkoxy(C₁-C₃)alkyl, hydroxy(C₃-C₈)cycloalkoxy(C₁-C₃)alkyl, (C₃-C₈)cycloalkyl(C₁-C₃)alkoxy(C₁-C₃)alkyl, halo(C₃-C₈)cycloalkyl(C₁-C₃)alkoxy(C₁-C₃)alkyl, hydroxy(C₃-C₈)cycloalkyl(C₁-C₃)alkoxy(C₁-C₃)alkyl, (C₁-C₈)alkylthio, halo(C₁-C₈)alkylthio, (C₃-C₈)cycloalkylthio, halo(C₃-C₈)cycloalkylthio, hydroxy(C₃-C₈)cycloalkylthio, (C₃-C₈)cycloalkyl(C₁-C₃)alkylthio, halo(C₃-C₈)cycloalkyl(C₁-C₃)alkylthio, hydroxy(C₃-C₈)cycloalkyl(C₁-C₃)alkylthio, (C₁-C₈)alkylthio(C₁-C₃)alkyl, halo(C₁-C₈)alkylthio(C₁-C₃)alkyl, (C₃-C₈)cycloalkylthio(C₁-C₃)alkyl, halo(C₃-C₈)cycloalkylthio(C₁-C₃)alkyl, hydroxy(C₃-C₈)cycloalkylthio(C₁-C₃)alkyl, (C₃-C₈)cycloalkyl(C₁-C₃)alkylthio(C₁-C₃)alkyl, halo(C₃-C₈)cycloalkyl(C₁-C₃)alkylthio(C₁-C₃)alkyl, hydroxy(C₃-C₈)cycloalkyl(C₁-C₃)alkylthio(C₁-C₃)alkyl and oxo; and
R¹⁰ is (C₁-C₆)alkyl or halo(C₁-C₆)alkyl.

2. The compound of claim 1, represented by the formula: 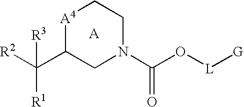
wherein:
Ring A is:
a) piperidine (A⁴ is CH₂);
b) azepane (A⁴ is CH₂CH₂);
c) morpholine (A⁴ is O); or
d) 1,4-oxazepane (A⁴ is CH₂O);
L is: 1) a linear (C₂-C₄)alkyl chain when G is —OH, —OR⁹, —NH₂, —NHR⁹, —NR⁹R¹⁰, —NHC(═NH)NH₂, or —NHC(═NH)NHR⁹; or 2) a linear (C₁-C₃)alkyl chain when G is —C(═NH)NH₂ or —C(═NH)NHR⁹;
L is substituted by 1-4 groups independently selected from R⁵, R⁶, R⁷, and R⁸;
each R⁵, R⁶, R⁷, and R⁸ is independently selected from:
1) hydrogen;
2) (C₁-C₁₂)alkyl, (C₃-C₈)cycloalkyl, (C₃-C₈)cycloalkyl(C₁-C₃)alkyl, (C₂-C₁₂)alkenyl, (C₅-C₈)cycloalkyl(C₁-C₃)alkenyl, (C₂-C₁₂)alkynyl, (C₃-C₈)cycloalkyl(C₁-C₃)alkynyl, (C₄-C₁₂)bicycloalkyl(C₁-C₃)alkyl, (C₈-C₁₄)tricycloalkyl(C₁-C₃)alkyl, (C₁-C₆)alkoxy(C₁-C₆)alkyl, (C₃-C₈)cycloalkoxy(C₁-C₃)alkyl, (C₁-C₆)alkylthio(C₁-C₆)alkyl, (C₃-C₈)cycloalkylthio(C₁-C₃)alkyl, saturated heterocyclyl, and saturated heterocyclyl(C₁-C₃)alkyl;
wherein each R⁵, R⁶, R⁷ and R⁸ is optionally and independently substituted by a group selected from: halogen, cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, halo(C₃-C₆)cycloalkoxy; and divalent sulfur atoms are optionally oxidized to sulfoxide or sulfone; and
3) phenyl, naphthyl, heteroaryl, phenyl(C₁-C₃)alkyl, naphthyl(C₁-C₃)alkyl, and heteroaryl(C₁-C₃)alkyl; each optionally substituted with 1 to 3 groups independently selected from:
fluoride, chloride, bromide, iodide, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkythio(C₁-C₆)alkyl, halo(C₄-C₈)-cycloalkylalkylthio(C₁-C₆)alkyl, (C₁-C₈)alkanesulfinyl(C₁-C₆)alkyl, (C₃-C₈)cycloalkanesulfinyl(C₁-C₆)alkyl, (C₄-C₈)cycloalkylalkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkanesulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkanesulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl(C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl, di(C₁-C₈)alkylaminocarboxy(C₁-C₆)alkyl, phenyl, napthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, napthyl(C₁-C₃)alkyl, heteroaryl(C₁-C₃)alkyl, and bicyclic heteroaryl(C₁-C₃)alkyl; wherein the aromatic and heteroaromatic groups are optionally substituted with 1 to 3 groups independently selected from: fluoride, chloride, cyano, (C₁-C₃)alkyl, halo(C₁-C₃)alkyl, (C₁-C₃)alkoxy, halo(C₁-C₃)-alkoxy, (C₁-C₃)alkanesulfonyl, and (C₁-C₃)alkoxycarbonyl; and
R⁹ is:
a) (C₁-C₁₂)alkyl, (C₄-C₁₂)cycloalkylalkyl, halo(C₁-C₁₂)alkyl, halo(C₄-C₁₂)cycloalkylalkyl, (C₂-C₁₂)alkenyl, (C₅-C₁₂)cycloalkylalkenyl, halo(C₂-C₁₂)alkenyl, halo(C₅-C₁₂)cycloalkylalkenyl, (C₂-C₁₂)alkynyl, (C₅-C₁₂)cycloalkylalkynyl, halo(C₂-C₁₂)alkynyl, halo(C₅-C₁₂)cycloalkylalkynyl, (C₁-C₆)alkoxy(C₁-C₆)alkyl, halo(C₁-C₆)alkoxy(C₁-C₆)alkyl, (C₁-C₆)alkylthio(C₁-C₆)alkyl, halo(C₁-C₆)alkylthio(C₁-C₆)alkyl, (C₁-C₆)alkanesulfinyl(C₁-C₆)alkyl, halo(C₁-C₆)alkanesulfinyl(C₁-C₆)alkyl, (C₁-C₆)alkanesulfonyl(C₁-C₆)alkyl, halo(C₁-C₆)alkanesulfonyl(C₁-C₆)alkyl, aminocarbonyl(C₁-C₆)alkyl, (C₁-C₆)alkylaminocarbonyl(C₁-C₆)alkyl, di(C₁-C₆)alkylaminocarbonyl(C₁-C₆)alkyl, cyano(C₁-C₆)alkyl, carboxy(C₁-C₆)alkyl, (C₁-C₆)alkoxycarbonyl(C₁-C₆)alkyl, saturated heterocyclyl, or saturated heterocyclyl(C₁-C₆)alkyl; or
b) phenyl, naphthyl, heteroaryl, phenyl(C₁-C₃)alkyl, naphthyl(C₁-C₃)alkyl, or heteroaryl(C₁-C₃)alkyl, each optionally and independently substituted by 1 to 3 groups selected from:
1) fluoride, chloride, bromide, iodide, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkylthio(C₁-C₆)alkyl, halo(C₄-C₈)cycloalkylalkylthio(C₁-C₆)alkyl, (C₁-C₈)alkanesulfinyl(C₁-C₆)alkyl, (C₃-C₈)cycloalkanesulfinyl(C₁-C₆)alkyl, (C₄-C₈)cycloalkylalkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkanesulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkanesulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl, (C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl and di(C₁-C₄)alkylaminocarboxy(C₁-C₆)alkyl; and
2) phenyl, napthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, napthyl(C₁-C₃)alkyl, heteroaryl(C₁-C₃)alkyl, and bicyclic heteroaryl(C₁-C₃)alkyl, each optionally substituted with 1 to 3 groups independently selected from: fluoride, chloride, cyano, (C₁-C₃)alkyl, halo(C₁-C₃)alkyl, halo(C₁-C₃)alkoxy, halo(C₁-C₃)alkoxy, (C₁-C₃)alkanesulfonyl, and (C₁-C₃)-alkoxycarbonyl;
or an enantiomer, diastereomer, or pharmaceutically acceptable salt thereof.

3. The compound of claim 2, represented by the formula: 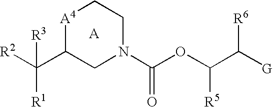
or an enantiomer, diastereomer, or pharmaceutically acceptable salt thereof.

4. The compound of claim 3, represented by the formula: 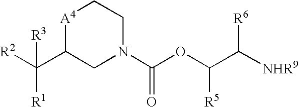
or an enantiomer, diastereomer, or pharmaceutically acceptable salt thereof.

5. The compound according to claim 4, represented by the formula: 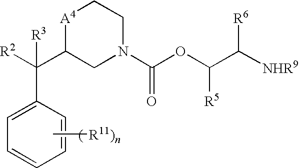
wherein n is an integer from 0 to 4;
or an enantiomer, diastereomer, or pharmaceutically acceptable salt thereof.

6. A compound according to claim 1, wherein one of R⁵ or R⁶ is hydrogen, but not both.

7. A compound according to claim 1, wherein R² is 4-methoxybutyl, 3-(methoxycarbonylamino)propyl or 2-(methoxycarbonylamino)ethoxy.

8. A compound according to claim 1, wherein R¹, where present, is phenyl, optionally substituted with n groups represented by R¹¹, wherein n is an integer from 0 to 3, and wherein R¹¹ is selected from: fluoride, chloride, nitro, cyano, (C₁-C₃)alkyl, halo(C₁-C₃)alkyl, (C₁-C₃)alkoxy, and halo(C₁-C₃)alkoxy.

9. The compound of claim 1, wherein:
R¹, where present, is:
a) (C₁-C₉)alkyl, (C₃-C₇)cycloalkyl, (C₄-C₉)cycloalkylalkyl, halo(C₁-C₉)alkyl, halo(C₃-C₇)cycloalkyl, halo(C₄-C₉)cycloalkylalkyl, or saturated heterocyclyl each optionally substituted with 1 to 3 groups independently selected from: fluoride, (C₁-C₃)alkyl, halo(C₁-C₃)alkyl, and oxo; or
b) phenyl, napthyl, heteroaryl or bicyclic heteroaryl, each substituted with n groups represented by R¹¹, wherein n is an integer from 0 to 3, and wherein each R¹¹ is independently selected from the groups consisting of:
1) fluoride, chloride, bromide, cyano, nitro, (C₁-C₆)alkyl, (C₃-C₆)cycloalkyl, (C₄-C₇)cycloalkylalkyl, (C₂-C₆)alkenyl, (C₅-C₆)cycloalkenyl, (C₅-C₈)cycloalkylalkenyl, (C₂-C₆)alkynyl, (C₃-C₆)cycloalkylethynyl, halo(C₁-C₆)alkyl, halo(C₃-C₆)cycloalkyl, halo(C₄-C₇)cycloalkylalkyl, halo(C₂-C₆)alkenyl, halo(C₃-C₆)alkynyl, halo(C₃-C₆)cycloalkylethynyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₃-C₆)alkenyloxy, and (C₁-C₆)alkanesulfonyl; or
2) phenyl, heteroaryl, phenoxy, heteroaryloxy, phenylthio, heteroarylthio, benzyl, heteroarylmethyl, benzyloxy, and heteroaryloxy, each optionally substituted with 1 to 3 groups independently selected from: fluoride, chloride, cyano, (C₁-C₃)alkyl, halo(C₁-C₃)alkyl, (C₁-C₃)alkoxy, halo(C₁-C₃)alkoxy, and aminocarbonyl;
R² is (C₁-C₈)alkyl, (C₄-C₈)cycloalkylalkyl, fluoro(C₁-C₈)alkyl, fluoro(C₄-C₈)cycloalkylalkyl, (C₁-C₈)alkoxy, (C₄-C₈)cycloalkylalkoxy, fluoro(C₁-C₈)alkoxy, hydroxy(C₁-C₈)alkyl, (C₁-C₅)alkoxy(C₁-C₅)alkyl, halo(C₁-C₅)alkylamino(C₁-C₅)alkyl, (C₁-C₅)alkoxy(C₁-C₅)hydroxyalkyl, (C₃-C₄)cycloalkoxy(C₁-C₅)alkyl, fluoro(C₁-C₅)alkoxy(C₁-C₅)alkyl, fluoro(C₃-C₄)cycloalkoxy(C₁-C₅)alkyl, (C₁-C₅)alkylthio(C₁-C₅)alkyl, (C₁-C₅)alkoxy(C₁-C₅)alkoxy, hydroxy(C₁-C₈)alkoxy, (C₃-C₄)cycloalkoxy(C₁-C₅)alkoxy, fluoro(C₁-C₅)alkoxy(C₁-C₅)alkoxy, fluoro(C₃-C₄)cycloalkoxy(C₁-C₅)alkoxy, (C₁-C₃)alkoxy(C₁-C₃)alkoxy(C₁-C₃)alkyl, fluoro(C₁-C₃)-alkoxy(C₁-C₃)alkoxy(C₁-C₃)alkyl, aminocarbonylamino(C₁-C₈)alkyl, aminocarbonylamino(C₁-C₈)alkoxy, (C₁-C₅)alkanoylamino(C₁-C₅)alkyl, (C₁-C₅)alkanoylamino(C₁-C₅)alkoxy, fluoro(C₁-C₅)alkanoylamino(C₁-C₅)alkyl, fluoro(C₁-C₅)alkanoylamino(C₁-C₅)alkoxy, (C₁-C₃)alkoxy(C₁-C₅)alkanoylamino(C₁-C₅)alkyl, (C₁-C₃)alkoxy(C₁-C₅)alkanoylamino(C₁-C₅)alkoxy, (C₃-C₄)cycloalkylcarbonylamino(C₁-C₅)alkyl, (C₃-C₄)cycloalkylcarbonylamino(C₁-C₅)alkoxy, aminosulfonylamino(C₁-C₈)alkyl, aminosulfonylamino(C₁-C₈)alkoxy, (C₁-C₅)alkanesulfonylamino(C₁-C₅)alkyl, (C₁-C₅)alkanesulfonylamino(C₁-C₅)alkoxy, formylamino(C₁-C₅)alkyl, formylamino(C₁-C₅)alkoxy, (C₁-C₅)alkoxycarbonylamino(C₁-C₅)alkyl, (C₁-C₅)alkoxycarbonylamino(C₁-C₅)alkoxy, (C₁-C₅)alkylaminocarbonylamino(C₁-C₅)alkyl, (C₁-C₅)alkylaminocarbonylamino(C₁-C₅)alkyl, di(C₁-C₅)alkylaminocarbonylamino(C₁-C₅)alkoxy, aminocarbonyl(C₁-C₅)alkyl, aminocarbonyl(C₁-C₅)alkoxy, (C₁-C₅)alkylaminocarbonyl(C₁-C₅)alkyl, (C₁-C₅)alkylaminocarbonyl(C₁-C₅)alkoxy, aminocarboxy(C₁-C₅)alkyl, aminocarboxy(C₁-C₅)alkoxy, (C₁-C₅)alkylaminocarboxy(C₁-C₅)alkyl, (C₁-C₅)alkylaminocarboxy(C₁-C₅)alkoxy, (C₁-C₈)alkoxycarbonylamino, (C₁-C₈)alkylaminocarbonylamino, (C₁-C₈)alkanoylamino, fluoro(C₁-C₈)alkoxycarbonylamino, fluoro(C₁-C₈)alkylaminocarbonylamino, (C₁-C₈)oxoalkyl, or fluoro(C₁-C₈)alkanoylamino;
R³ is —H, halogen, —OH, (C₁-C₄)alkanoylamino, or (C₁-C₃)alkoxy;
provided that when R³ is —OH or halogen, then R² is not (C₁-C₈)alkoxy, (C₄-C₈)cycloalkylalkoxy, fluoro(C₁-C₈)alkoxy, (C₁-C₅)alkoxy(C₁-C₅)alkoxy, hydroxy(C₁-C₈)alkoxy, (C₃-C₄)cycloalkoxy(C₁-C₅)alkoxy, fluoro(C₁-C₅)alkoxy(C₁-C₅)alkoxy, fluoro(C₃-C₄)cycloalkoxy(C₁-C₅)alkoxy, aminocarbonylamino(C₁-C₈)alkoxy, (C₁-C₅)alkanoylamino(C₁-C₅)alkoxy, fluoro(C₁-C₅)alkanoylamino(C₁-C₅)alkoxy, (C₁-C₃)alkoxy(C₁-C₅)alkanoylamino(C₁-C₅)alkoxy, (C₃-C₄)cycloalkylcarbonylamino(C₁-C₅)alkoxy, aminosulfonylamino(C₁-C₈)alkoxy, (C₁-C₅)alkanesulfonylamino(C₁-C₅)alkoxy, formylamino(C₁-C₅)alkoxy, (C₁-C₅)alkoxycarbonylamino(C₁-C₅)alkoxy, di(C₁-C₅)alkylaminocarbonylamino(C₁-C₅)alkoxy, aminocarbonyl(C₁-C₅)alkoxy, (C₁-C₅)alkylaminocarbonyl(C₁-C₅)alkoxy, aminocarboxy(C₁-C₅)alkoxy, (C₁-C₅)alkylaminocarboxy(C₁-C₅)alkoxy, (C₁-C₈)alkoxycarbonylamino, (C₁-C₈)alkylaminocarbonylamino, (C₁-C₈)alkanoylamino, fluoro(C₁-C₈)alkoxycarbonylamino, fluoro(C₁-C₈)alkylaminocarbonylamino, or fluoro(C₁-C₈)alkanoylamino;
A⁴ is CH₂, O, CH₂CH₂ or CH₂O;
R⁵ and R⁶ are independently selected from:
1) hydrogen; (C₁-C₁₀)alkyl, (C₃-C₇)cycloalkyl(C₁-C₂)alkyl, (C₄-C₁₀)bicycloalkyl(C₁-C₂)alkyl, (C₈-C₁₂)tricycloalkyl(C₁-C₂)alkyl, saturated heterocyclyl(C₁-C₃)alkyl, (C₁-C₆)alkoxy(C₁-C₆)alkyl, (C₃-C₇)cycloalkoxy(C₁-C₃)alkyl, or (C₁-C₅)alkylthio(C₁-C₅)alkyl; wherein each of these groups are optionally substituted by 1 to 3 groups independently selected from: halogen, cyano, hydroxy, (C₁-C₂)alkyl, (C₁-C₂)alkoxy, (C₃-C₆)cycloalkyl, (C₃-C₆)cycloalkoxy, halo(C₁-C₂)alkyl, halo(C₁-C₂)alkoxy, halo(C₃-C₆)cycloalkyl, and halo(C₃-C₆)cycloalkoxy; and
2) phenyl(C₁-C₂)alkyl or heteroaryl(C₁-C₂)alkyl, each optionally substituted with 1 to 3 groups independently selected from: fluoride, chloride, cyano, (C₁-C₃)alkyl, halo(C₁-C₃)alkyl, (C₁-C₃)alkoxy, and halo(C₁-C₃)alkoxy;
R⁹ is:
1) hydrogen, (C₁-C₆)alkyl, halo(C₁-C₆)alkyl, (C₄-C₁₀)cycloalkylalkyl, (C₁-C₅)alkoxy(C₁-C₅)alkyl, aminocarbonyl(C₁-C₅)alkyl, (C₁-C₆)alkylaminocarbonyl(C₁-C₆)alkyl, or di(C₁-C₆)alkylaminocarbonyl(C₁-C₆)alkyl; or
2) phenyl(C₁-C₂)alkyl, optionally substituted with 1 to 3 groups independently selected from: fluoride, chloride, cyano, (C₁-C₃)alkyl, halo(C₁-C₃)alkyl, (C₁-C₃)alkoxy, and halo(C₁-C₃)alkoxy.

10. The compound of claim 7, wherein:
R³ is —H, halogen, —OH, (C₁-C₄)alkanoylamino, or (C₁-C₃)alkoxy;
provided that when R³ is —OH or halogen, then R² is not (C₁-C₈)alkoxy, (C₄-C₈)cycloalkylalkoxy, fluoro(C₁-C₈)alkoxy, (C₁-C₅)alkoxy(C₁-C₅)alkoxy, hydroxy(C₁-C₈)alkoxy, (C₃-C₄)cycloalkoxy(C₁-C₅)alkoxy, fluoro(C₁-C₅)alkoxy(C₁-C₅)alkoxy, fluoro(C₃-C₄)cycloalkoxy(C₁-C₅)alkoxy, aminocarbonylamino(C₁-C₈)alkoxy, (C₁-C₅)alkanoylamino(C₁-C₅)alkoxy, fluoro(C₁-C₅)alkanoylamino(C₁-C₅)alkoxy, (C₁-C₃)alkoxy(C₁-C₅)alkanoylamino(C₁-C₅)alkoxy, (C₃-C₄)cycloalkylcarbonylamino(C₁-C₅)alkoxy, aminosulfonylamino(C₁-C₈)alkoxy, (C₁-C₅)alkanesulfonylamino(C₁-C₅)alkoxy, formylamino(C₁-C₅)alkoxy, (C₁-C₅)alkoxycarbonylamino(C₁-C₅)alkoxy, di(C₁-C₅)alkylaminocarbonylamino(C₁-C₅)alkoxy, aminocarbonyl(C₁-C₅)alkoxy, (C₁-C₅)alkylaminocarbonyl(C₁-C₅)alkoxy, aminocarboxy(C₁-C₅)alkoxy, (C₁-C₅)alkylaminocarboxy(C₁-C₅)alkoxy, (C₁-C₈)alkoxycarbonylamino, (C₁-C₈)alkylaminocarbonylamino, (C₁-C₈)alkanoylamino, fluoro(C₁-C₈)alkoxycarbonylamino, fluoro(C₁-C₈)alkylaminocarbonylamino, or fluoro(C₁-C₈)alkanoylamino;
A⁴ is CH₂, O, CH₂CH₂ or CH₂O;
R⁵ and R⁶ are independently selected from:
1) hydrogen; (C₁-C₁₀)alkyl, (C₃-C₇)cycloalkyl(C₁-C₂)alkyl, (C₄-C₁₀)bicycloalkyl(C₁-C₂)alkyl, (C₈-C₁₂)tricycloalkyl(C₁-C₂)alkyl, saturated heterocyclyl(C₁-C₃)alkyl, (C₁-C₆)alkoxy(C₁-C₆)alkyl, (C₃-C₇)cycloalkoxy(C₁-C₃)alkyl, or (C₁-C₅)alkylthio(C₁-C₅)alkyl; wherein each of these groups are optionally substituted by 1 to 3 groups independently selected from: halogen, cyano, hydroxy, (C₁-C₂)alkyl, (C₁-C₂)alkoxy, (C₃-C₆)cycloalkyl, (C₃-C₆)cycloalkoxy, halo(C₁-C₂)alkyl, halo(C₁-C₂)alkoxy, halo(C₃-C₆)cycloalkyl, and halo(C₃-C₆)cycloalkoxy; and
2) phenyl(C₁-C₂)alkyl or heteroaryl(C₁-C₂)alkyl, each optionally substituted with 1 to 3 groups independently selected from: fluoride, chloride, cyano, (C₁-C₃)alkyl, halo(C₁-C₃)alkyl, (C₁-C₃)alkoxy, and halo(C₁-C₃)alkoxy;
R⁹ is:
1) hydrogen, (C₁-C₆)alkyl, halo(C₁-C₆)alkyl, (C₄-C₁₀)cycloalkylalkyl, (C₁-C₅)alkoxy(C₁-C₅)alkyl, aminocarbonyl(C₁-C₅)alkyl, (C₁-C₆)alkylaminocarbonyl(C₁-C₆)alkyl, or di(C₁-C₆)alkylaminocarbonyl(C₁-C₆)alkyl; or
2) phenyl(C₁-C₂)alkyl, optionally substituted with 1 to 3 groups independently selected from: fluoride, chloride, cyano, (C₁-C₃)alkyl, halo(C₁-C₃)alkyl, (C₁-C₃)alkoxy, and halo(C₁-C₃)alkoxy.

11. A compound according to claim 1, wherein:
R¹ is: a saturated heterocycle, phenyl, or heteroaryl; wherein the saturated heterocycle is substituted with n groups, represented by R¹¹, wherein n is an integer from 0 to 3, and wherein each R¹¹ is independently selected from: fluoride, (C₁-C₃)alkyl, halo(C₁-C₃)alkyl, and oxo, and wherein the phenyl and heteroaryl are optionally and independently substituted with 1 to 3 groups selected from: halogen, nitro, cyano, (C₁-C₆)alkyl, halo(C₁-C₆)alkyl, (C₁-C₆)alkoxy, halo(C₁-C₆)alkoxy, phenyl and heteroaryl;
R² is: (C₁-C₅)alkoxy(C₁-C₅)alkyl, (C₁-C₅)alkoxy(C₁-C₅)hydroxyalkyl, (C₃-C₄)cycloalkoxy(C₁-C₅)alkyl, fluoro(C₁-C₅)alkoxy(C₁-C₅)alkyl, (C₁-C₅)alkoxy(C₁-C₅)alkoxy, fluoro(C₁-C₅)alkoxy(C₁-C₅)alkoxy, (C₁-C₃)alkoxy(C₁-C₃)alkoxy(C₁-C₃)alkyl, fluoro(C₁-C₃)alkoxy(C₁-C₃)alkoxy(C₁-C₃)alkyl, (C₁-C₅)alkanoylamino(C₁-C₅)alkyl, (C₁-C₅)alkanoylamino(C₁-C₅)alkoxy, fluoro(C₁-C₅)alkanoylamino(C₁-C₅)alkyl, fluoro(C₁-C₅)alkanoylamino(C₁-C₅)alkoxy, (C₁-C₃)alkoxy(C₁-C₅)alkanoylamino(C₁-C₅)alkyl, (C₁-C₃)alkoxy(C₁-C₅)alkanoylamino(C₁-C₅)alkoxy, (C₁-C₅)alkoxycarbonylamino(C₁-C₅)alkyl, (C₁-C₅)alkoxycarbonylamino(C₁-C₅)alkoxy, (C₁-C₅)alkylaminocarbonylamino(C₁-C₅)alkyl, di(C₁-C₅)alkylaminocarbonylamino(C₁-C₅)alkoxy, (C₁-C₈)alkylaminocarbonylamino, fluoro(C₁-C₈)alkoxycarbonylamino, or (C₁-C₈)oxoalkyl;
R³ is hydrogen, fluoride, chloride, —OH or (C₁-C₃)alkoxy;
provided that when R³ is —OH, —F, or —Cl, then R² is not (C₁-C₅)alkoxy(C₁-C₅)alkoxy, fluoro(C₁-C₅)alkoxy(C₁-C₅)alkoxy, (C₁-C₅)alkanoylamino(C₁-C₅)alkoxy, fluoro(C₁-C₅)alkanoylamino(C₁-C₅)alkoxy, (C₁-C₃)alkoxy(C₁-C₅)alkanoylamino(C₁-C₅)alkoxy, (C₁-C₅)alkoxycarbonylamino(C₁-C₅)alkoxy, di(C₁-C₅)alkylaminocarbonylamino(C₁-C₅)alkoxy, (C₁-C₈)alkylaminocarbonylamino, or fluoro(C₁-C₈)alkoxycarbonylamino;
A⁴ is CH₂ or O;
R⁵ and R⁶ are each independently hydrogen, (C₃-C₇)cycloalkyl(C₁-C₂)alkyl, saturated heterocyclyl(C₁-C₃)alkyl, (C₃-C₇)cycloalkoxy(C₁-C₃)alkyl, or heteroaryl(C1-C2)alkyl, wherein each group is optionally and individually substituted with 1 to 3 groups selected from: fluoride, chloride, cyano, (C₁-C₃)alkyl, halo(C₁-C₃)alkyl, (C₁-C₃)alkoxy, or halo(C₁-C₃)alkoxy; and
R⁹ is: hydrogen, (C₁-C₃)alkyl, halo(C₁-C₃)alkyl, (C₄-C₈)cycloalkylalkyl, or (C₁-C₃)alkoxy(C₁-C₃)alkyl.

12. A compound according to claim 7, wherein:
R³ is hydrogen, fluoride, chloride, —OH or (C₁-C₃)alkoxy;
provided that when R³ is —OH, —F, or —Cl, then R² is not (C₁-C₅)alkoxy(C₁-C₅)alkoxy, fluoro(C₁-C₅)alkoxy(C₁-C₅)alkoxy, (C₁-C₅)alkanoylamino(C₁-C₅)alkoxy, fluoro(C₁-C₅)alkanoylamino(C₁-C₅)alkoxy, (C₁-C₃)alkoxy(C₁-C₅)alkanoylamino(C₁-C₅)alkoxy, (C₁-C₅)alkoxycarbonylamino(C₁-C₅)alkoxy, di(C₁-C₅)alkylaminocarbonylamino(C₁-C₅)alkoxy, (C₁-C₈)alkylaminocarbonylamino, or fluoro(C₁-C₈)alkoxycarbonylamino;
A⁴ is CH₂ or O;
R⁵ and R⁶ are each independently hydrogen, (C₃-C₇)cycloalkyl(C₁-C₂)alkyl, saturated heterocyclyl(C₁-C₃)alkyl, (C₃-C₇)cycloalkoxy(C₁-C₃)alkyl, or heteroaryl(C1-C2)alkyl, wherein each group is optionally and individually substituted with 1 to 3 groups selected from: fluoride, chloride, cyano, (C₁-C₃)alkyl, halo(C₁-C₃)alkyl, (C₁-C₃)alkoxy, or halo(C₁-C₃)alkoxy; and
R⁹ is: hydrogen, (C₁-C₃)alkyl, halo(C₁-C₃)alkyl, (C₄-C₈)cycloalkylalkyl, or (C₁-C₃)alkoxy(C₁-C₃)alkyl.

13. A compound according to claim 1, wherein
R¹, where present, is phenyl optionally substituted with 1 to 3 substituents independently selected from fluoride, chloride and methyl;
R² is 4-methoxybutyl, 3-(methoxycarbonylamino)propyl or 2-(methoxycarbonylamino)ethoxy;
R³ is H or OH; provided that when R² is 2-(methoxycarbonylamino)ethoxy R³ is not —OH;
A⁴ is CH₂ or O;
R⁵ is cyclohexylmethyl or 3-(tetrahydropyranyl)methyl and R⁶ is H, or R⁵ is H and R⁶ is cyclohexylmethyl or 3-(tetrahydropyranyl)methyl; and
R⁹ is H or methyl.

14. A compound of claim 1, according to the formula: 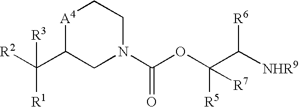
wherein:
A⁴ is CH₂, O, CH₂CH₂ or CH₂O; and
R⁷ taken together with R⁹ and their intervening atoms form a saturated 3-, 4-, 5-, 6-, or 7-membered “L-G ring” comprising 3 to 7 carbon atoms, and 1 or 2 hetero atoms selected from 1 nitrogen atom, 0 or 1 oxygen atoms, and 0 or 1 sulfur atoms; said L-G ring being optionally substituted with 1 to 4 groups selected from: halogen, (C₁-C₈)alkyl, halo(C₁-C₈)alkyl, (C₃-C₈)cycloalkyl, (C₁-C₂)alkyl(C₃-C₆)cycloalkyl, halo(C₃-C₈)cycloalkyl, hydroxy(C₃-C₈)cycloalkyl, (C₃-C₈)cycloalkyl(C₁-C₃)alkyl, halo(C₃-C₈)cycloalkyl(C₁-C₃)alkyl, hydroxy(C₃-C₈)cycloalkyl(C₁-C₃)alkyl, (C₁-C₈)alkoxy, halo(C₁-C₈)alkoxy, (C₃-C₈)cycloalkoxy, halo(C₃-C₈)cycloalkoxy, hydroxy(C₃-C₈)cycloalkoxy, (C₁-C₈)alkoxy(C₁-C₃)alkyl, halo(C₁-C₈)alkoxy(C₁-C₃)alkyl, (C₃-C₈)cycloalkoxy(C₁-C₃)alkyl, halo(C₃-C₈)cycloalkoxy(C₁-C₃)alkyl, hydroxy(C₃-C₈)cycloalkoxy(C₁-C₃)alkyl, (C₃-C₈)cycloalkyl(C₁-C₃)alkoxy(C₁-C₃)alkyl, halo(C₃-C₈)cycloalkyl(C₁-C₃)alkoxy(C₁-C₃)alkyl, hydroxy(C₃-C₈)cycloalkyl(C₁-C₃)alkoxy(C₁-C₃)alkyl, (C₁-C₈)alkylthio, halo(C₁-C₈)alkylthio, (C₃-C₈)cycloalkylthio, halo(C₃-C₈)cycloalkylthio, hydroxy(C₃-C₈)cycloalkylthio, (C₃-C₈)cycloalkyl(C₁-C₃)alkylthio, halo(C₃-C₈)cycloalkyl(C₁-C₃)alkylthio, hydroxy(C₃-C₈)cycloalkyl(C₁-C₃)alkylthio, (C₁-C₈)alkylthio(C₁-C₃)alkyl, halo(C₁-C₈)alkylthio(C₁-C₃)alkyl, (C₃-C₈)cycloalkylthio(C₁-C₃)alkyl, halo(C₃-C₈)cycloalkylthio(C₁-C₃)alkyl, hydroxy(C₃-C₈)cycloalkylthio(C₁-C₃)alkyl, (C₃-C₈)cycloalkyl(C₁-C₃)alkylthio(C₁-C₃)alkyl, halo(C₃-C₈)cycloalkyl(C₁-C₃)alkylthio(C₁-C₃)alkyl, hydroxy(C₃-C₈)cycloalkyl(C₁-C₃)alkylthio(C₁-C₃)alkyl and oxo;
or an enantiomer, diastereomer, or pharmaceutically acceptable salt thereof.

15. A compound according to claim 14, according to the formula: 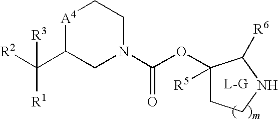
wherein m is either 1 or 2; and
said L-G ring being optionally substituted with 1 to 4 groups selected from: halogen, (C₁-C₈)alkyl, halo(C₁-C₈)alkyl, (C₃-C₈)cycloalkyl, (C₁-C₂)alkyl(C₃-C₆)cycloalkyl, halo(C₃-C₈)cycloalkyl, hydroxy(C₃-C₈)cycloalkyl, (C₃-C₈)cycloalkyl(C₁-C₃)alkyl, halo(C₃-C₈)cycloalkyl(C₁-C₃)alkyl, hydroxy(C₃-C₈)cycloalkyl(C₁-C₃)alkyl, (C₁-C₈)alkoxy, halo(C₁-C₈)alkoxy, (C₃-C₈)cycloalkoxy, halo(C₃-C₈)cycloalkoxy, hydroxy(C₃-C₈)cycloalkoxy, (C₁-C₈)alkoxy(C₁-C₃)alkyl, halo(C₁-C₈)alkoxy(C₁-C₃)alkyl, (C₃-C₈)cycloalkoxy(C₁-C₃)alkyl, halo(C₃-C₈)cycloalkoxy(C₁-C₃)alkyl, hydroxy(C₃-C₈)cycloalkoxy(C₁-C₃)alkyl, (C₃-C₈)cycloalkyl(C₁-C₃)alkoxy(C₁-C₃)alkyl, halo(C₃-C₈)cycloalkyl(C₁-C₃)alkoxy(C₁-C₃)alkyl, hydroxy(C₃-C₈)cycloalkyl(C₁-C₃)alkoxy(C₁-C₃)alkyl, (C₁-C₈)alkylthio, halo(C₁-C₈)alkylthio, (C₃-C₈)cycloalkylthio, halo(C₃-C₈)cycloalkylthio, hydroxy(C₃-C₈)cycloalkylthio, (C₃-C₈)cycloalkyl(C₁-C₃)alkylthio, halo(C₃-C₈)cycloalkyl(C₁-C₃)alkylthio, hydroxy(C₃-C₈)cycloalkyl(C₁-C₃)alkylthio, (C₁-C₈)alkylthio(C₁-C₃)alkyl, halo(C₁-C₈)alkylthio(C₁-C₃)alkyl, (C₃-C₈)cycloalkylthio(C₁-C₃)alkyl, halo(C₃-C₈)cycloalkylthio(C₁-C₃)alkyl, hydroxy(C₃-C₈)cycloalkylthio(C₁-C₃)alkyl, (C₃-C₈)cycloalkyl(C₁-C₃)alkylthio(C₁-C₃)alkyl, halo(C₃-C₈)cycloalkyl(C₁-C₃)alkylthio(C₁-C₃)alkyl, hydroxy(C₃-C₈)cycloalkyl(C₁-C₃)alkylthio(C₁-C₃)alkyl and oxo;
or an enantiomer, diastereomer, or pharmaceutically acceptable salt thereof.

16. The compound according to claim 15, represented by the formula: 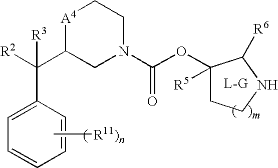
wherein n is an integer from 0 to 4; and
said L-G ring being optionally substituted with 1 to 4 groups selected from: halogen, (C₁-C₈)alkyl, halo(C₁-C₈)alkyl, (C₃-C₈)cycloalkyl, (C₁-C₂)alkyl(C₃-C₆)cycloalkyl, halo(C₃-C₈)cycloalkyl, hydroxy(C₃-C₈)cycloalkyl, (C₃-C₈)cycloalkyl(C₁-C₃)alkyl, halo(C₃-C₈)cycloalkyl(C₁-C₃)alkyl, hydroxy(C₃-C₈)cycloalkyl(C₁-C₃)alkyl, (C₁-C₈)alkoxy, halo(C₁-C₈)alkoxy, (C₃-C₈)cycloalkoxy, halo(C₃-C₈)cycloalkoxy, hydroxy(C₃-C₈)cycloalkoxy, (C₁-C₈)alkoxy(C₁-C₃)alkyl, halo(C₁-C₈)alkoxy(C₁-C₃)alkyl, (C₃-C₈)cycloalkoxy(C₁-C₃)alkyl, halo(C₃-C₈)cycloalkoxy(C₁-C₃)alkyl, hydroxy(C₃-C₈)cycloalkoxy(C₁-C₃)alkyl, (C₃-C₈)cycloalkyl(C₁-C₃)alkoxy(C₁-C₃)alkyl, halo(C₃-C₈)cycloalkyl(C₁-C₃)alkoxy(C₁-C₃)alkyl, hydroxy(C₃-C₈)cycloalkyl(C₁-C₃)alkoxy(C₁-C₃)alkyl, (C₁-C₈)alkylthio, halo(C₁-C₈)alkylthio, (C₃-C₈)cycloalkylthio, halo(C₃-C₈)cycloalkylthio, hydroxy(C₃-C₈)cycloalkylthio, (C₃-C₈)cycloalkyl(C₁-C₃)alkylthio, halo(C₃-C₈)cycloalkyl(C₁-C₃)alkylthio, hydroxy(C₃-C₈)cycloalkyl(C₁-C₃)alkylthio, (C₁-C₈)alkylthio(C₁-C₃)alkyl, halo(C₁-C₈)alkylthio(C₁-C₃)alkyl, (C₃-C₈)cycloalkylthio(C₁-C₃)alkyl, halo(C₃-C₈)cycloalkylthio(C₁-C₃)alkyl, hydroxy(C₃-C₈)cycloalkylthio(C₁-C₃)alkyl, (C₃-C₈)cycloalkyl(C₁-C₃)alkylthio(C₁-C₃)alkyl, halo(C₃-C₈)cycloalkyl(C₁-C₃)alkylthio(C₁-C₃)alkyl, hydroxy(C₃-C₈)cycloalkyl(C₁-C₃)alkylthio(C₁-C₃)alkyl and oxo;
or an enantiomer, diastereomer, or pharmaceutically acceptable salt thereof.

17. A compound according to claim 14, wherein at least one of R⁵ or R⁶ is hydrogen.

18. A compound according to claim 14, wherein R² is 4-methoxybutyl, 3-(methoxycarbonylamino)propyl or 2-(methoxycarbonylamino)ethoxy.

19. A compound according to claim 14, wherein:
R¹, where present, is:
1) (C₁-C₉)alkyl, (C₃-C₇)cycloalkyl, (C₄-C₉)cycloalkylalkyl, halo(C₁-C₉)alkyl, halo(C₃-C₇)cycloalkyl, halo(C₄-C₉)cycloalkylalkyl, or saturated heterocyclyl, each optionally substituted with 1 to 3 groups independently selected from: fluoride, (C₁-C₃)alkyl, halo(C₁-C₃)alkyl, and oxo; or
2) phenyl, napthyl, heteroaryl, or bicyclic heteroaryl each optionally substituted with 1 to 3 groups independently selected from:
a) fluoride, chloride, bromide, cyano, nitro, (C₁-C₆)alkyl, (C₃-C₆)cycloalkyl, (C₄-C₇)cycloalkylalkyl, (C₂-C₆)alkenyl, (C₅-C₆)cycloalkenyl, (C₅-C₈)cycloalkylalkenyl, (C₂-C₆)alkynyl, (C₃-C₆)cycloalkylethynyl, halo(C₁-C₆)alkyl, halo(C₃-C₆)cycloalkyl, halo(C₄-C₇)-cycloalkylalkyl, halo(C₂-C₆)alkenyl, halo(C₃-C₆)alkynyl, halo(C₃-C₆)cycloalkylethynyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₃-C₆)alkenyloxy, or (C₁-C₆)alkanesulfonyl; and
b) phenyl, heteroaryl, phenoxy, heteroaryloxy, phenylthio, heteroarylthio, benzyl, heteroarylmethyl, benzyloxy, or heteroaryloxy, each optionally substituted with 1 to 3 groups independently selected from: fluoride, chloride, cyano, (C₁-C₃)alkyl, halo(C₁-C₃)alkyl, (C₁-C₃)alkoxy, halo(C₁-C₃)alkoxy, and aminocarbonyl;
R², where present, is (C₁-C₈)alkyl, (C₄-C₈)cycloalkylalkyl, fluoro(C₁-C₈)alkyl, fluoro(C₄-C₈)cycloalkylalkyl, (C₁-C₈)alkoxy, (C₄-C₈)cycloalkylalkoxy, fluoro(C₁-C₈)alkoxy, hydroxy(C₁-C₈)alkyl, (C₁-C₅)alkoxy(C₁-C₅)alkyl, halo(C₁-C₅)alkylamino(C₁-C₅)alkyl, (C₁-C₅)alkoxy(C₁-C₅)hydroxyalkyl, (C₃-C₄)cycloalkoxy(C₁-C₅)alkyl, fluoro(C₁-C₅)alkoxy(C₁-C₅)alkyl, fluoro(C₃-C₄)cycloalkoxy(C₁-C₅)alkyl, (C₁-C₅)alkylthio(C₁-C₅)alkyl, (C₁-C₅)alkoxy(C₁-C₅)alkoxy, hydroxy(C₁-C₈)alkoxy, (C₃-C₄)cycloalkoxy(C₁-C₅)alkoxy, fluoro(C₁-C₅)alkoxy(C₁-C₅)alkoxy, fluoro(C₃-C₄)cycloalkoxy(C₁-C₅)alkoxy, (C₁-C₃)alkoxy(C₁-C₃)alkoxy(C₁-C₃)alkyl, fluoro(C₁-C₃)alkoxy(C₁-C₃)alkoxy(C₁-C₃)alkyl, aminocarbonylamino(C₁-C₈)alkyl, aminocarbonylamino(C₁-C₈)alkoxy, (C₁-C₅)alkanoylamino(C₁-C₅)alkyl, (C₁-C₅)alkanoylamino(C₁-C₅)alkoxy, fluoro(C₁-C₅)alkanoylamino(C₁-C₅)alkyl, fluoro(C₁-C₅)alkanoylamino(C₁-C₅)alkoxy, (C₁-C₃)alkoxy(C₁-C₅)alkanoylamino(C₁-C₅)alkyl, (C₁-C₃)alkoxy(C₁-C₅)alkanoylamino(C₁-C₅)alkoxy, (C₃-C₄)cycloalkylcarbonylamino(C₁-C₅)alkyl, (C₃-C₄)cycloalkylcarbonylamino(C₁-C₅)alkoxy, aminosulfonylamino(C₁-C₈)alkyl, aminosulfonylamino(C₁-C₈)alkoxy, (C₁-C₅)alkanesulfonylamino(C₁-C₅)alkyl, (C₁-C₅)alkanesulfonylamino(C₁-C₅)alkoxy, formylamino(C₁-C₅)alkyl, formylamino(C₁-C₅)alkoxy, (C₁-C₅)alkoxycarbonylamino(C₁-C₅)alkyl, (C₁-C₅)alkoxycarbonylamino(C₁-C₅)alkoxy, (C₁-C₅)alkylaminocarbonylamino(C₁-C₅)alkyl, di(C₁-C₅)alkylaminocarbonylamino(C₁-C₅)alkoxy, aminocarbonyl(C₁-C₅)alkyl, aminocarbonyl(C₁-C₅)alkoxy, (C₁-C₅)alkylaminocarbonyl(C₁-C₅)alkyl, (C₁-C₅)alkylaminocarbonyl(C₁-C₅)alkoxy, aminocarboxy(C₁-C₅)alkyl, aminocarboxy(C₁-C₅)alkoxy, (C₁-C₅)alkylaminocarboxy(C₁-C₅)alkyl, (C₁-C₅)alkylaminocarboxy(C₁-C₅)alkoxy, (C₁-C₈)alkoxycarbonylamino, (C₁-C₈)alkylaminocarbonylamino, (C₁-C₈)alkanoylamino, fluoro(C₁-C₈)alkoxycarbonylamino, fluoro(C₁-C₈)alkylaminocarbonylamino, fluoro(C₁-C₈)alkanoylamino, or (C₁-C₈)oxoalkyl;
R³ is H, halogen, —OH, (C₁-C₄)alkanoylamino, or (C₁-C₃)alkoxy;
provided that when R³ is —OH or halogen, then R² is not (C₁-C₈)alkoxy, (C₄-C₈)cycloalkylalkoxy, fluoro(C₁-C₈)alkoxy, (C₁-C₅)alkoxy(C₁-C₅)alkoxy, hydroxy(C₁-C₈)alkoxy, (C₃-C₄)cycloalkoxy(C₁-C₅)alkoxy, fluoro(C₁-C₅)alkoxy(C₁-C₅)alkoxy, fluoro(C₃-C₄)cycloalkoxy(C₁-C₅)alkoxy, aminocarbonylamino(C₁-C₈)alkoxy, (C₁-C₅)alkanoylamino(C₁-C₅)alkoxy, fluoro(C₁-C₅)alkanoylamino(C₁-C₅)alkoxy, (C₁-C₃)alkoxy(C₁-C₅)alkanoylamino(C₁-C₅)alkoxy, (C₃-C₄)cycloalkylcarbonylamino(C₁-C₅)alkoxy, aminosulfonylamino(C₁-C₈)alkoxy, (C₁-C₅)alkanesulfonylamino(C₁-C₅)alkoxy, formylamino(C₁-C₅)alkoxy, (C₁-C₅)alkoxycarbonylamino(C₁-C₅)alkoxy, di(C₁-C₅)alkylaminocarbonylamino(C₁-C₅)alkoxy, aminocarbonyl(C₁-C₅)alkoxy, (C₁-C₅)alkylaminocarbonyl(C₁-C₅)alkoxy, aminocarboxy(C₁-C₅)alkoxy, (C₁-C₅)alkylaminocarboxy(C₁-C₅)alkoxy, (C₁-C₈)alkoxycarbonylamino, (C₁-C₈)alkylaminocarbonylamino, (C₁-C₈)alkanoylamino, fluoro(C₁-C₈)alkoxycarbonylamino, fluoro(C₁-C₈)alkylaminocarbonylamino, or fluoro(C₁-C₈)alkanoylamino;
A⁴ is CH₂, O, CH₂CH₂ or CH₂O;
R⁵ and R⁶ are each independently selected from:
1) hydrogen;
2) (C₁-C₁₀)alkyl, (C₃-C₇)cycloalkyl(C₁-C₂)alkyl, (C₄-C₁₀)bicycloalkyl(C₁-C₂)alkyl, (C₈-C₁₂)tricycloalkyl(C₁-C₂)alkyl, saturated heterocyclyl(C₁-C₂)alkyl, (C₁-C₆)alkoxy(C₁-C₆)alkyl, (C₃-C₇)cycloalkoxy(C₁-C₃)alkyl, (C₁-C₅)alkylthio(C₁-C₅)alkyl, or saturated heterocyclyl(C₁-C₃)alkyl, wherein each of these groups are optionally substituted by 1 to 3 groups independently selected from: halogen, cyano, hydroxy, (C₁-C₂)alkyl, (C₁-C₂)alkoxy, (C₃-C₆)cycloalkyl, (C₃-C₆)cycloalkoxy, halo(C₁-C₂)alkyl, halo(C₁-C₂)alkoxy, halo(C₃-C₆)cycloalkyl, and halo(C₃-C₆)cycloalkoxy; and
3) phenyl(C₁-C₂)alkyl or heteroaryl(C₁-C₂)alkyl, wherein each group is optionally substituted with 1 to 3 groups independently selected from: fluoride, chloride, cyano, (C₁-C₃)alkyl, halo(C₁-C₃)alkyl, (C₁-C₃)alkoxy, and halo(C₁-C₃)alkoxy.

20. A compound according to claim 14, wherein:
R¹, where present, is: a saturated heterocycle, phenyl, or heteroaryl; wherein the saturated heterocycle is optionally substituted with 1 to 3 groups independently selected from: fluoride, (C₁-C₃)alkyl, halo(C₁-C₃)alkyl, and oxo, and wherein the phenyl and heteroaryl are optionally and independently substituted with n groups represented by R¹¹, wherein n is an integer from 0-3, and wherein each R¹¹ is independently selected from: halogen, nitro, cyano, (C₁-C₆)alkyl, halo(C₁-C₆)alkyl, (C₁-C₆)alkoxy, halo(C₁-C₆)alkoxy, phenyl and heteroaryl;
R², where present, is: (C₁-C₅)alkoxy(C₁-C₅)alkyl, (C₁-C₅)alkoxy(C₁-C₅)hydroxyalkyl, (C₃-C₄)cycloalkoxy(C₁-C₅)alkyl, fluoro(C₁-C₅)alkoxy(C₁-C₅)alkyl, (C₁-C₅)alkoxy(C₁-C₅)alkoxy, fluoro(C₁-C₅)alkoxy(C₁-C₅)alkoxy, (C₁-C₃)alkoxy(C₁-C₃)alkoxy(C₁-C₃)alkyl, fluoro(C₁-C₃)alkoxy(C₁-C₃)alkoxy(C₁-C₃)alkyl, (C₁-C₅)alkanoylamino(C₁-C₅)alkyl, (C₁-C₅)alkanoylamino(C₁-C₅)alkoxy, fluoro(C₁-C₅)alkanoylamino(C₁-C₅)alkyl, fluoro(C₁-C₅)alkanoylamino(C₁-C₅)alkoxy, (C₁-C₃)alkoxy(C₁-C₅)alkanoylamino(C₁-C₅)alkyl, (C₁-C₃)alkoxy(C₁-C₅)alkanoylamino(C₁-C₅)alkoxy, (C₁-C₅)alkoxycarbonylamino(C₁-C₅)alkyl, (C₁-C₅)alkoxycarbonylamino(C₁-C₅)alkoxy, (C₁-C₅)alkylaminocarbonylamino(C₁-C₅)alkyl, di(C₁-C₅)alkylaminocarbonylamino(C₁-C₅)alkoxy, (C₁-C₈)alkylaminocarbonylamino, fluoro(C₁-C₈)alkoxycarbonylamino, or (C₁-C₈)oxoalkyl;
R³ is hydrogen, fluoride, chloride, —OH or (C1-C3)alkoxy;
provided that when R³ is —OH, —F, or —Cl, then R² is not (C₁-C₅)alkoxy(C₁-C₅)alkoxy, fluoro(C₁-C₅)alkoxy(C₁-C₅)alkoxy, (C₁-C₅)alkanoylamino(C₁-C₅)alkoxy, fluoro(C₁-C₅)alkanoylamino(C₁-C₅)alkoxy, (C₁-C₃)alkoxy(C₁-C₅)alkanoylamino(C₁-C₅)alkoxy, (C₁-C₅)alkoxycarbonylamino(C₁-C₅)alkoxy, di(C₁-C₅)alkylaminocarbonylamino(C₁-C₅)alkoxy, (C₁-C₈)alkylaminocarbonylamino, or fluoro(C₁-C₈)alkoxycarbonylamino;
A⁴ is CH₂ or O;
R⁵ and R⁶ are each individually: hydrogen, (C₃-C₇)cycloalkyl(C₁-C₂)alkyl, saturated heterocyclyl(C₁-C₃)alkyl, (C₃-C₇)cycloalkoxy(C₁-C₃)alkyl, or heteroaryl(C1-C2)alkyl, wherein each group is optionally and individually substituted with 1 to 3 groups selected from: fluoride, chloride, cyano, (C₁-C₃)alkyl, halo(C₁-C₃)alkyl, (C₁-C₃)alkoxy, or halo(C₁-C₃)alkoxy; and
the substituents on “L-G ring” are selected from: fluoride, (C₁-C₈)alkyl, halo(C₁-C₈)alkyl, (C₃-C₈)cycloalkyl, halo(C₃-C₈)cycloalkyl, hydroxy(C₃-C₈)cycloalkyl, (C₃-C₈)cycloalkyl(C₁-C₃)alkyl, halo(C₃-C₈)cycloalkyl(C₁-C₃)alkyl, hydroxy(C₃-C₈)cycloalkyl(C₁-C₃)alkyl, (C₁-C₈)alkoxy, halo(C₁-C₈)alkoxy, (C₃-C₈)cycloalkoxy, halo(C₃-C₈)cycloalkoxy, hydroxy(C₃-C₈)cycloalkoxy, (C₁-C₈)alkoxy(C₁-C₃)alkyl, halo(C₁-C₈)alkoxy(C₁-C₃)alkyl, (C₃-C₈)cycloalkoxy(C₁-C₃)alkyl, halo(C₃-C₈)cycloalkoxy(C₁-C₃)alkyl, hydroxy(C₃-C₈)cycloalkoxy(C₁-C₃)alkyl, (C₃-C₈)cycloalkyl(C₁-C₃)alkoxy(C₁-C₃)alkyl, halo(C₃-C₈)cycloalkyl(C₁-C₃)alkoxy(C₁-C₃)alkyl, and hydroxy(C₃-C₈)cycloalkyl(C₁-C₃)alkoxy(C₁-C₃)alkyl.

21. A compound according to claim 14, wherein:
R¹ is phenyl optionally substituted with 1 to 3 substituents independently selected from fluoride, chloride and methyl;
R² is 4-methoxybutyl, 3-(methoxycarbonylamino)propyl or 2-(methoxycarbonylamino)ethoxy;
R³ is —H or —OH;
provided that when R² is 2-(methoxycarbonylamino)ethoxy, then R³ is not —OH;
A⁴ is CH₂ or O;
R⁵ is H, or cyclohexylmethyl;
R⁶ is H, or cyclohexylmethyl;
allowing both R⁵ and R⁶ to be H simultaneously; and
the “L-G ring” is optionally substituted with one group selected from: (C₃-C₆)alkyl, (C₃-C₆)cycloalkyl, and (C₃-C₆)cycloalkyl(C₁-C₂)alkyl.

22. A compound selected from the group consisting of:
(R)-((S)-1-cyclohexyl-3-(methylamino)propan-2-yl) 3-((S)-1-(3-chloro-2-fluorophenyl)-1-hydroxy-5-methoxypentyl)piperidine-1-carboxylate;
(R)-((S)-1-cyclohexyl-3-(methylamino)propan-2-yl) 3-((S)-1-(3-chlorophenyl)-1-hydroxy-4-(methoxycarbonylamino)butyl)piperidine-1-carboxylate;
(R)-((S)-1-cyclohexyl-3-(methylamino)propan-2-yl) 2-((R)-1-(3-chloro-2-fluorophenyl)-1-hydroxy-5-methoxypentyl)morpholine-4-carboxylate;
(R)-((S)-2-amino-3-cyclohexylpropyl) 3-((S)-1-(3-chloro-2-fluorophenyl)-1-hydroxy-5-methoxypentyl)piperidine-1-carboxylate;
(R)-((S)-2-amino-3-((R)-tetrahydro-2H-pyran-3-yl)propyl) 3-((R)-(3-chlorophenyl)(2-(methoxycarbonylamino)ethoxy)methyl)piperidine-1-carboxylate;
(R)-((R)-1-cyclohexyl-3-(methylamino)propan-2-yl) 3-((S)-1-(3-chlorophenyl)-1-hydroxy-4-(methoxycarbonylamino)butyl)piperidine-1-carboxylate;
(R)-((3SR,4RS)-4-isobutylpiperidin-3-yl) 3-((R)-(3-chlorophenyl)(2-(methoxycarbonylamino)ethoxy)methyl)piperidine-1-carboxylate;
(R)-((S)-3-(cyclohexylmethyl)pyrrolidin-3-yl) 3-((R)-(3-chlorophenyl)(2-(methoxycarbonylamino)ethoxy)methyl)piperidine-1-carboxylate;
(R)-((R)-3-(cyclohexylmethyl)pyrrolidin-3-yl) 3-((R)-(3-chlorophenyl)(2-(methoxycarbonylamino)ethoxy)methyl)piperidine-1-carboxylate;
(R)-((R)-1-cyclohexyl-3-(methylamino)propan-2-yl) 3-((S)-1-(3-chloro-2-fluorophenyl)-1-hydroxy-5-methoxypentyl)piperidine-1-carboxylate; and
(R)-((R)-1-cyclohexyl-3-(methylamino)propan-2-yl) 2-((R)-1-(3-chloro-2-fluorophenyl)-1-hydroxy-5-methoxypentyl)morpholine-4-carboxylate; or enantiomers, diastereomers, or pharmaceutically acceptable salts thereof;

23. A compound selected from the list consisting of:
(R)-((S)-1-cyclohexyl-3-(methylamino)propan-2-yl) 3-((S)-1-(3-chloro-2-fluorophenyl)-1-hydroxy-5-methoxypentyl)piperidine-1-carboxylate;
(R)-((S)-1-cyclohexyl-3-(methylamino)propan-2-yl) 3-((S)-1-(3-chlorophenyl)-1-hydroxy-4-(methoxycarbonylamino)butyl)piperidine-1-carboxylate;
(R)-((S)-1-cyclohexyl-3-(methylamino)propan-2-yl) 2-((R)-1-(3-chloro-2-fluorophenyl)-1-hydroxy-5-methoxypentyl)morpholine-4-carboxylate;
(R)-((S)-2-amino-3-cyclohexylpropyl) 3-((S)-1-(3-chloro-2-fluorophenyl)-1-hydroxy-5-methoxypentyl)piperidine-1-carboxylate; and
(R)-((S)-2-amino-3-((R)-tetrahydro-2H-pyran-3-yl)propyl) 3-((R)-(3-chlorophenyl)(2-(methoxycarbonylamino)ethoxy)methyl)piperidine-1-carboxylate; or enantiomers, diastereomers, or pharmaceutically acceptable salts thereof.

24. A compound selected from the group consisting of:
(R)-((S)-1-cyclohexyl-3-(methylamino)propan-2-yl) 3-((S)-1-(3-chlorophenyl)-1-hydroxy-4-(methoxycarbonylamino)butyl)piperidine-1-carboxylate; and
(R)-((S)-2-amino-3-((R)-tetrahydro-2H-pyran-3-yl)propyl) 3-((R)-(3-chlorophenyl)(2-(methoxycarbonylamino)ethoxy)methyl)piperidine-1-carboxylate; or an enantiomer, diastereomer, or pharmaceutically acceptable salt thereof.

25. A pharmaceutical composition comprising a compound of claim 1, or an enantiomer, diastereomer, or salt thereof and a pharmaceutically acceptable carrier or excipient.

26. The pharmaceutical composition of claim 25, further comprising an additional agent selected from the group consisting of α-blockers, β-blockers, calcium channel blockers, diuretics, angiotensin converting enzyme (ACE) inhibitors, dual ACE and neutral endopeptidase (NEP) inhibitors, angiotensin-receptor blockers (ARBs), aldosterone synthase inhibitors, aldosterone-receptor antagonists, and endothelin receptor antagonists.

27.-37. (canceled)

38. A pharmaceutical composition comprising a compound of claim 22, and a pharmaceutically acceptable carrier or excipient.

39. A pharmaceutical composition comprising a compound of claim 23, and a pharmaceutically acceptable carrier or excipient.

40. A pharmaceutical composition comprising a compound of claim 24, and a pharmaceutically acceptable carrier or excipient.
